(19) United States
(12) Patent Application Publication
(10) Pub. No.: US 2005/0186588 A1
(43) Pub. Date: Aug. 25, 2005

(54) Direct nucleic acid detection in bodily fluids

(75) Inventors: Victor Lyamichev, Madison, WI (US); Andrew A. Lukowiak, Stoughton, WI (US); Nancy Jarvis, Madison, WI (US); David Kurensky, Milwaukee, WI (US); Hatim T. Allawi, Madison, WI (US); Scott M. Law, Madison, WI (US); Chad Gerber, Middleton, WI (US); Robert Roeven, Stoughton, WI (US); Jeff G. Hall, Madison, WI (US)

Correspondence Address: MEDLEN & CARROLL, LLP, Suite 350, 101 Howard Street, San Francisco, CA, 94105, US

(73) Assignee: Third Wave Technologies, Inc.

(21) Appl. No.: 10/967,711

(22) Filed: Oct. 18, 2004

Related U.S. Application Data
(60) Provisional application No. 60/511,955, filed on Oct. 16, 2003.
(60) Provisional application No. 60/549,527, filed on Mar. 2, 2004.
(60) Provisional application No. 60/551,669, filed on Mar. 9, 2004.

(51) Int. Cl. C12Q 1/68; C12P 19/34

(52) U.S. Cl. 435/6; 435/91.2

(57) ABSTRACT

The present invention provides methods and routines for developing and optimizing nucleic acid detection assays for use in basic research, clinical research, and for the development of clinical detection assays. In particular, the present invention provides methods for designing oligonucleotide primers to be used in multiplex amplification reactions. The present invention also provides methods to optimize multiplex amplification reactions. The present invention also provides methods for combined target and signal generation assays.

The present Application claims priority to the following Provisional Applications: U.S. Provisional Application 60/511,955, filed Oct. 16, 2003; U.S. Provisional Application 60/549,527, filed Mar. 2, 2004; and U.S. Provisional Application 60/554,669, filed Mar. 9, 2004; all of which are herein incorporated by reference. The present Application incorporates by reference U.S. Provisional Application Ser. No. 60/511,955, filed Oct. 16, 2003.

### FIELD OF THE INVENTION

The present invention provides methods for combining target amplification reactions with signal amplification reactions to achieve rapid and sensitive detection of small quantities of nucleic acids, particularly in unpurified bodily fluids (e.g. blood). The present invention also provides methods to optimize multiplex amplification reactions. The present invention also provides methods to perform highly multiplexed PCR in combination with the INVADER assay. The present invention further provides methods to perform PCR in combination with the INVADER assay in a single reaction vessel (e.g. using unpurified bodily fluids such as blood) without the need for intervening manipulations or reagent additions.

### BACKGROUND

With the completion of the nucleic acid sequencing of the human genome, the demand for fast, reliable, cost-effective and user-friendly tests for genomics research and related drug design efforts has greatly increased. A number of institutions are actively mining the available genetic sequence information to identify correlations between genes, gene expression and phenotypes (e.g., disease states, metabolic responses, and the like). These analyses include an attempt to characterize the effect of gene mutations and genetic and gene expression heterogeneity in individuals and populations.

Advances in nucleic acid extraction and amplification have greatly expanded the types of biological samples from which genetic material may be obtained. In particular, Polymerase Chain Reaction (PCR) has made it possible to obtain sufficient quantities of DNA from fixed tissue samples, archaeological specimens, and quantities of many types of cells that number in the single digits. Similarly, large-scale SNP genotyping projects require quantities of genomic DNA that may be difficult to obtain from standard biological samples. One approach to addressing this issue relies on PCR-based target amplification.

While PCR enables analysis of minute quantities of nucleic acids, its practical application in a number of settings and for a number of types of problems remains problematic. Because small quantities of target nucleic acid are readily amplified by the reaction, PCR applications are highly susceptible to carry-over contamination from assay to assay. This vulnerability often necessitates the establishment of dedicated facilities or the configuration of workflows that minimize the number of post-amplification manipulations. In some cases, specialized instrumentation that allows reactions to be monitored in real-time without opening reaction vessels is used.

What is needed, then, is a method that limits the need for target amplification by maximizing signal generation from small amounts of amplified sequences.

### SUMMARY OF THE INVENTION

The present invention provides methods and routines for developing and optimizing nucleic acid detection assays for use in basic research, clinical research, and for the development of clinical detection assays.

In some embodiments, the present invention provides methods comprising; a) providing target sequence information for at least Y target sequences, wherein each of the target sequences comprises; i) a footprint region, ii) a 5′ region immediately upstream of the footprint region, and iii) a 3′ region immediately downstream of the footprint region, and b) processing the target sequence information such that a primer set is generated, wherein the primer set comprises a forward and a reverse primer sequence for each of the at least Y target sequences, wherein each of the forward and reverse primer sequences comprises a nucleic acid sequence represented by 5′-N[x]-N[x−1]- . . . -N[4]-N[3]-N[2]-N[1]-3′, wherein N represents a nucleotide base, x is at least 6, N[1] is nucleotide A or C, and N[2]-N[1]-3′ of each of the forward and reverse primers is not complementary to N[2]-N[1]-3′ of any of the forward and reverse primers in the primer set.

In other embodiments, the present invention provides methods comprising; a) providing target sequence information for at least Y target sequences, wherein each of the target sequences comprises; i) a footprint region, ii) a 5′ region immediately upstream of the footprint region, and iii) a 3′ region immediately downstream of the footprint region, and b) processing the target sequence information such that a primer set is generated, wherein the primer set comprises a forward and a reverse primer sequence for each of the at least Y target sequences, wherein each of the forward and reverse primer sequences comprises a nucleic acid sequence represented by 5′-N[x]-N[x−1]- . . . -N[4]-N[3]-N[2]-N[1]-3′, wherein N represents a nucleotide base, x is at least 6, N[1] is nucleotide G or T, and N[2]-N[1]-3′ of each of the forward and reverse primers is not complementary to N[2]-N[1]-3′ of any of the forward and reverse primers in the primer set.

In particular embodiments, a method comprising; a) providing target sequence information for at least Y target sequences, wherein each of the target sequences comprises; i) a footprint region, ii) a 5′ region immediately upstream of the footprint region, and iii) a 3′ region immediately downstream of the footprint region, and b) processing the target sequence information such that a primer set is generated, wherein the primer set comprises; i) a forward primer sequence identical to at least a portion of the 5′ region for each of the Y target sequences, and ii) a reverse primer sequence identical to at least a portion of a complementary sequence of the 3′ region for each of the at least Y target sequences, wherein each of the forward and reverse primer sequences comprises a nucleic acid sequence represented by 5′-N[x]-N[x−1]- . . . -N[4]-N[3]-N[2]-N[1]-3′, wherein N represents a nucleotide base, x is at least 6, N[1] is nucleotide A or C, and N[2]-N[1]-3′ of each of the forward and reverse primers is not complementary to N[2]-N[1]-3′ of any of the forward and reverse primers in the primer set.

In other embodiments, the present invention provides methods comprising a) providing target sequence information for at least Y target sequences, wherein each of the target sequences comprises; i) a footprint region, ii) a 5′ region immediately upstream of the footprint region, and iii) a 3′ region immediately downstream of the footprint region, and b) processing the target sequence information such that a primer set is generated, wherein the primer set comprises; i) a forward primer sequence identical to at least a portion of the 5′ region for each of the Y target sequences, and ii) a reverse primer sequence identical to at least a portion of a complementary sequence of the 3′ region for each of the at least Y target sequences, wherein each of the forward and reverse primer sequences comprises a nucleic acid sequence represented by 5′-N[x]-N[x−1]- . . . -N[4]-N[3]-N[2]-N[1]-3′, wherein N represents a nucleotide base, x is at least 6, N[1] is nucleotide G or T, and N[2]-N[1]-3′ of each of the forward and reverse primers is not complementary to N[2]-N[1]-3′ of any of the forward and reverse primers in the primer set.

In particular embodiments, the present invention provides methods comprising a) providing target sequence information for at least Y target sequences, wherein each of the target sequences comprises a single nucleotide polymorphism, b) determining where on each of the target sequences one or more assay probes would hybridize in order to detect the single nucleotide polymorphism such that a footprint region is located on each of the target sequences, and c) processing the target sequence information such that a primer set is generated, wherein the primer set comprises; i) a forward primer sequence identical to at least a portion of the target sequence immediately 5′ of the footprint region for each of the Y target sequences, and ii) a reverse primer sequence identical to at least a portion of a complementary sequence of the target sequence immediately 3′ of the footprint region for each of the at least Y target sequences, wherein each of the forward and reverse primer sequences comprises a nucleic acid sequence represented by 5′-N[x]-N[x−1]- . . . -N[4]-N[3]-N[2]-N[1]-3′, wherein N represents a nucleotide base, x is at least 6, N[1] is nucleotide A or C, and N[2]-N[1]-3′ of each of the forward and reverse primers is not complementary to N[2]-N[1]-3′ of any of the forward and reverse primers in the primer set.

In some embodiments, the present invention provides methods comprising a) providing target sequence information for at least Y target sequences, wherein each of the target sequences comprises a single nucleotide polymorphism, b) determining where on each of the target sequences one or more assay probes would hybridize in order to detect the single nucleotide polymorphism such that a footprint region is located on each of the target sequences, and c) processing the target sequence information such that a primer set is generated, wherein the primer set comprises; i) a forward primer sequence identical to at least a portion of the target sequence immediately 5′ of the footprint region for each of the Y target sequences, and ii) a reverse primer sequence identical to at least a portion of a complementary sequence of the target sequence immediately 3′ of the footprint region for each of the at least Y target sequences, wherein each of the forward and reverse primer sequences comprises a nucleic acid sequence represented by 5′-N[x]-N[x−1]- . . . -N[4]-N[3]-N[2]-N[1]-3′, wherein N represents a nucleotide base, x is at least 6, N[1] is nucleotide T or G, and N[2]-N[1]-3′ of each of the forward and reverse primers is not complementary to N[2]-N[1]-3′ of any of the forward and reverse primers in the primer set.

In certain embodiments, the primer set is configured for performing a multiplex PCR reaction that amplifies at least Y amplicons, wherein each of the amplicons is defined by the position of the forward and reverse primers. In other embodiments, the primer set is generated as digital or printed sequence information. In some embodiments, the primer set is generated as physical primer oligonucleotides.

In certain embodiments, N[3]-N[2]-N[1]-3′ of each of the forward and reverse primers is not complementary to N[3]-N[2]-N[1]-3′ of any of the forward and reverse primers in the primer set. In other embodiments, the processing comprises initially selecting N[1] for each of the forward primers as the most 3′ A or C in the 5′ region. In certain embodiments, the processing comprises initially selecting N[1] for each of the forward primers as the most 3′ G or T in the 5′ region. In some embodiments, the processing comprises initially selecting N[1] for each of the forward primers as the most 3′ A or C in the 5′ region, and wherein the processing further comprises changing the N[1] to the next most 3′ A or C in the 5′ region for the forward primer sequences that fail the requirement that each of the forward primer's N[2]-N[1]-3′ is not complementary to N[2]-N[1]-3′ of any of the forward and reverse primers in the primer set.

In other embodiments, the processing comprises initially selecting N[1] for each of the reverse primers as the most 3′ A or C in the complement of the 3′ region. In some embodiments, the processing comprises initially selecting N[1] for each of the reverse primers as the most 3′ G or T in the complement of the 3′ region. In further embodiments, the processing comprises initially selecting N[1] for each of the reverse primers as the most 3′ A or C in the 3′ region, and wherein the processing further comprises changing the N[1] to the next most 3′ A or C in the 3′ region for the reverse primer sequences that fail the requirement that each of the reverse primer's N[2]-N[1]-3′ is not complementary to N[2]-N[1]-3′ of any of the forward and reverse primers in the primer set.

In particular embodiments, the footprint region comprises a single nucleotide polymorphism. In some embodiments, the footprint comprises a mutation. In some embodiments, the footprint region for each of the target sequences comprises a portion of the target sequence that hybridizes to one or more assay probes configured to detect the single nucleotide polymorphism. In certain embodiments, the footprint is this region where the probes hybridize. In other embodiments, the footprint further includes additional nucleotides on either end.

In some embodiments, the processing further comprises selecting N[5]-N[4]-N[3]-N[2]-N[1]-3′ for each of the forward and reverse primers such that less than 80 percent homology with a assay component sequence is present. In preferred embodiments, the assay component is a FRET probe sequence. In certain embodiments, the target sequence is about 300-500 base pairs in length, or about 200-600 base pair in length. In certain embodiments, Y is an integer between 2 and 500, or between 2-10,000.

In certain embodiments, the processing comprises selecting x for each of the forward and reverse primers such that each of the forward and reverse primers has a melting temperature with respect to the target sequence of approximately 50 degrees Celsius (e.g. 50 degrees, Celsius, or at least 50 degrees Celsius, and no more than 55 degrees Celsius). In preferred embodiments, the melting temperature of a primer (when hybridized to the target sequence) is at least 50 degrees Celsius, but at least 10 degrees different than a selected detection assay's optimal reaction temperature.

In some embodiments, the forward and reverse primer pair optimized concentrations are determined for the primer set. In other embodiments, the processing is automated. In further embodiments, the processing is automated with a processor.

In other embodiments, the present invention provides a kit comprising the primer set generated by the methods of the present invention, and at least one other component. (e.g. cleavage agent, polymerase, INVADER oligonucleotide). In certain embodiments, the present invention provides compositions comprising the primers and primer sets generated by the methods of the present invention.

In particular embodiments, the present invention provides methods comprising; a) providing; i) a user interface configured to receive sequence data, ii) a computer system having stored therein a multiplex PCR primer software application, and b) transmitting the sequence data from the user interface to the computer system, wherein the sequence data comprises target sequence information for at least Y target sequences, wherein each of the target sequences comprises; i) a footprint region, ii) a 5′ region immediately upstream of the footprint region, and iii) a 3′ region immediately downstream of the footprint region, and c) processing the target sequence information with the multiplex PCR primer pair software application to generate a primer set, wherein the primer set comprises; i) a forward primer sequence identical to at least a portion of the target sequence immediately 5′ of the footprint region for each of the Y target sequences, and ii) a reverse primer sequence identical to at least a portion of a complementary sequence of the target sequence immediately 3′ of the footprint region for each of the at least Y target sequences, wherein each of the forward and reverse primer sequences comprises a nucleic acid sequence represented by 5′-N[x]-N[x−1]- . . . -N[4]-N[3]-N[2]-N[1]-3′, wherein N represents a nucleotide base, x is at least 6, N[1] is nucleotide A or C, and N[2]-N[1]-3′ of each of the forward and reverse primers is not complementary to N[2]-N[1]-3′ of any of the forward and reverse primers in the primer set.

In some embodiments, the present invention provides methods comprising; a) providing; i) a user interface configured to receive sequence data, ii) a computer system having stored therein a multiplex PCR primer software application, and b) transmitting the sequence data from the user interface to the computer system, wherein the sequence data comprises target sequence information for at least Y target sequences, wherein each of the target sequences comprises; i) a footprint region, ii) a 5′ region immediately upstream of the footprint region, and iii) a 3′ region immediately downstream of the footprint region, and c) processing the target sequence information with the multiplex PCR primer pair software application to generate a primer set, wherein the primer set comprises; i) a forward primer sequence identical to at least a portion of the target sequence immediately 5′ of the footprint region for each of the Y target sequences, and ii) a reverse primer sequence identical to at least a portion of a complementary sequence of the target sequence immediately 3′ of the footprint region for each of the at least Y target sequences, wherein each of the forward and reverse primer sequences comprises a nucleic acid sequence represented by 5′-N[x]-N[x−1]- . . . -N[4]-N[3]-N[2]-N[1]-3′, wherein N represents a nucleotide base, x is at least 6, N[1] is nucleotide G or T, and N[2]-N[1]-3′ of each of the forward and reverse primers is not complementary to N[2]-N[1]-3′ of any of the forward and reverse primers in the primer set.

In certain embodiments, the present invention provides systems comprising; a) a computer system configured to receive data from a user interface, wherein the user interface is configured to receive sequence data, wherein the sequence data comprises target sequence information for at least Y target sequences, wherein each of the target sequences comprises; i) a footprint region, ii) a 5′ region immediately upstream of the footprint region, and iii) a 3′ region immediately downstream of the footprint region, b) a multiplex PCR primer pair software application operably linked to the user interface, wherein the multiplex PCR primer software application is configured to process the target sequence information to generate a primer set, wherein the primer set comprises; i) a forward primer sequence identical to at least a portion of the target sequence immediately 5′ of the footprint region for each of the Y target sequences, and ii) a reverse primer sequence identical to at least a portion of a complementary sequence of the target sequence immediately 3′ of the footprint region for each of the at least Y target sequences, wherein each of the forward and reverse primer sequences comprises a nucleic acid sequence represented by 5′-N[x]-N[x−1]- . . . -N[4]-N[3]-N[2]-N[1]-3′, wherein N represents a nucleotide base, x is at least 6, N[1] is nucleotide A or C, and N[2]-N[1]-3′ of each of the forward and reverse primers is not complementary to N[2]-N[1]-3′ of any of the forward and reverse primers in the primer set, and c) a computer system having stored therein the multiplex PCR primer pair software application, wherein the computer system comprises computer memory and a computer processor.

In other embodiments, the present invention provides systems comprising; a) a computer system configured to receive data from a user interface, wherein the user interface is configured to receive sequence data, wherein the sequence data comprises target sequence information for at least Y target sequences, wherein each of the target sequences comprises; i) a footprint region, ii) a 5′ region immediately upstream of the footprint region, and iii) a 3′ region immediately downstream of the footprint region, b) a multiplex PCR primer pair software application operably linked to the user interface, wherein the multiplex PCR primer software application is configured to process the target sequence information to generate a primer set, wherein the primer set comprises; i) a forward primer sequence identical to at least a portion of the target sequence immediately 5′ of the footprint region for each of the Y target sequences, and ii) a reverse primer sequence identical to at least a portion of a complementary sequence of the target sequence immediately 3′ of the footprint region for each of the at least Y target sequences, wherein each of the forward and reverse primer sequences comprises a nucleic acid sequence represented by 5′-N[x]-N[x−1]- . . . -N[4]-N[3]-N[2]-N[1]-3′, wherein N represents a nucleotide base, x is at least 6, N[1] is nucleotide G or T, and N[2]-N[1]-3′ of each of the forward and reverse primers is not complementary to N[2]-N[1]-3′ of any of the forward and reverse primers in the primer set, and c) a computer system having stored therein the multiplex PCR primer pair software application, wherein the computer system comprises computer memory and a computer processor. In certain embodiments, the computer system is configured to return the primer set to the user interface.

In some embodiments, the present invention provides methods for conducting target and signal amplification reactions in a single reaction vessel. In some preferred embodiments, the target amplification reactions are PCR reactions. In some particularly preferred embodiments, the signal amplification reactions are invasive cleavage (INVADER) assays. In other embodiments, reagents for the combined target and signal amplification reactions are added prior to initiation of either reaction. In certain embodiments, the target amplification reactions are terminated after 20 cycles. In some preferred embodiments, the target amplification reactions are terminated after 15 cycles. In some particularly preferred embodiments, the target amplification reactions are terminated after 11 cycles. In some embodiments, some components are predispensed to a reaction vessel prior to addition of the remaining assay components. In preferred embodiments, the predispensed reagents are dried in the reaction vessel. In particularly preferred embodiments, the predispensed reagents comprise one or more INVADER assay reagents. In some embodiments, the reaction vessel comprises a microfluidic card. In preferred embodiments, the reaction vessel comprises a microfluidic card configured for centrifugal or centripetal distribution or manipulation of fluid reactions and reaction components.

In still other embodiments, the present invention provides methods and compositions for conducting multi-dye multiplex FRET INVADER assays, e.g., in a single reaction or reaction vessel. In some preferred embodiments, the multiplex FRET assays are carried out on synthetic targets. In other preferred embodiments, the multiplex FRET assays are carried out on nucleic acid fragment targets, e.g., PCR amplicons. In some particularly preferred embodiments, multiplex FRET assays are carried out on genomic DNA targets. In still other preferred embodiments, multiplex FRET assays are carried out on RNA targets. In some particularly preferred embodiments, the multiplex FRET assays are tetraplex reactions.

In some embodiments one or more the INVADER assay reagents may be provided in a predispensed format (i.e., premeasured for use in a step of the procedure without re-measurement or re-dispensing). In some embodiments, selected INVADER assay reagent components are mixed and predispensed together. In other embodiments, In preferred embodiments, predispensed assay reagent components are predispensed and are provided in a reaction vessel (including but not limited to a reaction tube or a well, as in, e.g., a microtiter plate). In particularly preferred embodiments, predispensed INVADER assay reagent components are dried down (e.g., desiccated or lyophilized) in a reaction vessel.

In some embodiments, the INVADER assay reagents are provided as a kit. As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. As used herein, the term “fragmented kit” refers to delivery systems comprising two or more separate containers that each contains a subportion of the total kit components. The containers may be delivered to the intended recipient together or separately. For example, a first container may contain an enzyme for use in an assay, while a second container contains oligonucleotides. The term “fragmented kit” is intended to encompass kits containing Analyte specific reagents (ASR's) regulated under section 520(e) of the Federal Food, Drug, and Cosmetic Act, but are not limited thereto. Indeed, any delivery system comprising two or more separate containers that each contains a subportion of the total kit components are included in the term “fragmented kit.” In contrast, a “combined kit” refers to a delivery system containing all of the components of a reaction assay in a single container (e.g., in a single box housing each of the desired components). The term “kit” includes both fragmented and combined kits.

In some embodiments, the present invention provides INVADER assay reagent kits comprising one or more of the components necessary for practicing the present invention. For example, the present invention provides kits for storing or delivering the enzymes and/or the reaction components necessary to practice an INVADER assay. The kit may include any and all components necessary or desired for assays including, but not limited to, the reagents themselves, buffers, control reagents (e.g., tissue samples, positive and negative control target oligonucleotides, etc.), solid supports, labels, written and/or pictorial instructions and product information, inhibitors, labeling and/or detection reagents, package environmental controls (e.g., ice, desiccants, etc.), and the like. In some embodiments, the kits provide a sub-set of the required components, wherein it is expected that the user will supply the remaining components. In some embodiments, the kits comprise two or more separate containers wherein each container houses a subset of the components to be delivered. For example, a first container (e.g., box) may contain an enzyme (e.g., structure specific cleavage enzyme in a suitable storage buffer and container), while a second box may contain oligonucleotides (e.g., INVADER oligonucleotides, probe oligonucleotides, control target oligonucleotides, etc.).

### DESCRIPTION OF THE FIGURES

The following figures form part of the present specification and are included to further demonstrate certain aspects and embodiments of the present invention. The invention may be better understood by reference to one or more of these figures in combination with the description of specific embodiments presented herein.

FIG. 1 shows a schematic diagram of an embodiment of the INVADER assay. In the primary reaction, the target molecule (hatched rectangle) forms the overlap-flap structure with the invasive probe (open rectangle) and the primary probe which includes the target-specific region (open rectangle) and the 5′ flap (filled rectangle). The overlap-flap is cleaved by the structure-specific 5′ nuclease. The cleavage site of the overlap-flap structure shown by the arrow is located after the 5′ terminal nucleotide of the target-specific region of the primary probe. For SNP identification, the overlap between the probes is positioned opposite the polymorphic site (X). If the X nucleotide is not complementary to the primary probe, no specific cleavage occurs. In the secondary reaction, the cleaved 5′ flap forms the overlap-flap structure with FRET cassette (gray line) labeled with a dye (D) and quencher (O). Cleavage of the FRET cassette by the 5′ nuclease releases the unquenched dye. The semicircular arrows indicate the oligonucleotide turnover process essential for signal amplification. A similar cascade (not shown) is used for detection of the alternative SNP nucleotide in the biplex INVADER assay.

FIG. 2 is a graph showing the dependence of the logarithm of the amplification factor lgF on the number of PCR cycles n for the PCR 5.

FIG. 3A is a graph showing the effect of primer concentration c on lgF for the PCR 1 (●), PCR 2(◯), PCR 4 (▪), and PCR 5 (□).

FIG. 3B is a graph showing the relationship between ln(2−F^{0.05}) and c using the data shown in 3A.

FIG. 4 shows scatter plots of the net FAM and RED INVADER assay signals for eight genomic DNA samples in reactions as described in Example 7.

FIG. 5 shows the net RED fluorescence signal normalized per allele for the 161 successful INVADER assays as a function of PCR target length, in reactions as described in Example 7.

FIG. 6 shows scatter plots of the net FAM and RED signals for the eight DNA samples in reactions as described in Example 7.

FIG. 7 shows a graph displaying the results of a combined target and signal amplification reaction according to the methods of Example 8.

FIG. 8 shows a flow chart outlining the steps that may be performed in order to generated a primer set useful in multiplex PCR.

FIG. 9 shows a graph displaying the results of a combined multiplex target and signal amplification reaction according to the methods of Example 8.

FIG. 10 shows a graph displaying the results of a tetraplex INVADER assay as described in Example 9.

FIGS. 11A-11G show graphs displaying the results of INVADER assay detection of multiplex PCR amplified target DNA in a microfluidic card.

FIGS. 12A-12G show graphs displaying the results of combined multiplex PCR and INVADER assay signal amplification reactions in a microfluidic card.

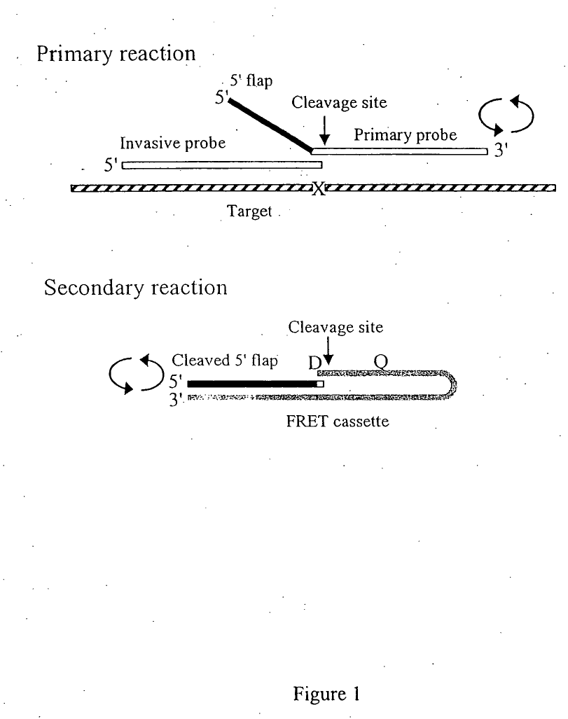

### DEFINITIONS

To facilitate an understanding of the present invention, a number of terms and phrases are defined below:

As used herein, the terms “SNP,” “SNPs” or “single nucleotide polymorphisms” refer to single base changes at a specific location in an organism's (e.g., a human) genome. “SNPs” can be located in a portion of a genome that does not code for a gene. Alternatively, a “SNP” may be located in the coding region of a gene. In this case, the “SNP” may alter the structure and function of the RNA or the protein with which it is associated.

As used herein, the term “allele” refers to a variant form of a given sequence (e.g., including but not limited to, genes containing one or more SNPs). A large number of genes are present in multiple allelic forms in a population. A diploid organism carrying two different alleles of a gene is said to be heterozygous for that gene, whereas a homozygote carries two copies of the same allele.

As used herein, the term “linkage” refers to the proximity of two or more markers (e.g., genes) on a chromosome.

As used herein, the term “allele frequency” refers to the frequency of occurrence of a given allele (e.g., a sequence containing a SNP) in given population (e.g., a specific gender, race, or ethnic group). Certain populations may contain a given allele within a higher percent of its members than other populations. For example, a particular mutation in the breast cancer gene called BRCA1 was found to be present in one percent of the general Jewish population. In comparison, the percentage of people in the general U.S. population that have any mutation in BRCA1 has been estimated to be between 0.1 to 0.6 percent. Two additional mutations, one in the BRCA1 gene and one in another breast cancer gene called BRCA2, have a greater prevalence in the Ashkenazi Jewish population, bringing the overall risk for carrying one of these three mutations to 2.3 percent.

As used herein, the term “in silico analysis” refers to analysis performed using computer processors and computer memory. For example, “in silico SNP analysis” refers to the analysis of SNP data using computer processors and memory.

As used herein, the term “genotype” refers to the actual genetic make-up of an organism (e.g., in terms of the particular alleles carried at a genetic locus). Expression of the genotype gives rise to an organism's physical appearance and characteristics—the “phenotype.”

As used herein, the term “locus” refers to the position of a gene or any other characterized sequence on a chromosome.

As used herein the term “disease” or “disease state” refers to a deviation from the condition regarded as normal or average for members of a species, and which is detrimental to an affected individual under conditions that are not inimical to the majority of individuals of that species (e.g., diarrhea, nausea, fever, pain, and inflammation etc).

As used herein, the term “treatment” in reference to a medical course of action refer to steps or actions taken with respect to an affected individual as a consequence of a suspected, anticipated, or existing disease state, or wherein there is a risk or suspected risk of a disease state. Treatment may be provided in anticipation of or in response to a disease state or suspicion of a disease state, and may include, but is not limited to preventative, ameliorative, palliative or curative steps. The term “therapy” refers to a particular course of treatment.

The term “gene” refers to a nucleic acid (e.g., DNA) sequence that comprises coding sequences necessary for the production of a polypeptide, RNA (e.g., rRNA, tRNA, etc.), or precursor. The polypeptide, RNA, or precursor can be encoded by a full length coding sequence or by any portion of the coding sequence so long as the desired activity or functional properties (e.g., ligand binding, signal transduction, etc.) of the full-length or fragment are retained. The term also encompasses the coding region of a structural gene and the including sequences located adjacent to the coding region on both the 5′ and 3′ ends for a distance of about 1 kb on either end such that the gene corresponds to the length of the full-length mRNA. The sequences that are located 5′ of the coding region and which are present on the mRNA are referred to as 5′ untranslated sequences. The sequences that are located 3′ or downstream of the coding region and that are present on the mRNA are referred to as 3′ untranslated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments included when a gene is transcribed into heterogeneous nuclear RNA (hnRNA); introns may contain regulatory elements such as enhancers. Introns are removed or “spliced out” from the nuclear or primary transcript; introns therefore are generally absent in the messenger RNA (mRNA) transcript. The mRNA functions during translation to specify the sequence or order of amino acids in a nascent polypeptide. Variations (e.g., mutations, SNPS, insertions, deletions) in transcribed portions of genes are reflected in, and can generally be detected in corresponding portions of the produced RNAs (e.g., hnRNAs, mRNAs, rRNAs, tRNAs).

Where the phrase “amino acid sequence” is recited herein to refer to an amino acid sequence of a naturally occurring protein molecule, amino acid sequence and like terms, such as polypeptide or protein are not meant to limit the amino acid sequence to the complete, native amino acid sequence associated with the recited protein molecule.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

The term “wild-type” refers to a gene or gene product that has the characteristics of that gene or gene product when isolated from a naturally occurring source. A wild-type gene is that which is most frequently observed in a population and is thus arbitrarily designed the “normal” or “wild-type” form of the gene. In contrast, the terms “modified,” “mutant,” and “variant” refer to a gene or gene product that displays modifications in sequence and or functional properties (i.e., altered characteristics) when compared to the wild-type gene or gene product. It is noted that naturally-occurring mutants can be isolated; these are identified by the fact that they have altered characteristics when compared to the wild-type gene or gene product.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. In this case, the DNA sequence thus codes for the amino acid sequence.

DNA and RNA molecules are said to have “5′ ends” and “3′ ends” because mononucleotides are reacted to make oligonucleotides or polynucleotides in a manner such that the 5′ phosphate of one mononucleotide pentose ring is attached to the 3′ oxygen of its neighbor in one direction via a phosphodiester linkage. Therefore, an end of an oligonucleotides or polynucleotide, referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide or polynucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements that direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

As used herein, the terms “an oligonucleotide having a nucleotide sequence encoding a gene” and “polynucleotide having a nucleotide sequence encoding a gene,” means a nucleic acid sequence comprising the coding region of a gene or, in other words, the nucleic acid sequence that encodes a gene product. The coding region may be present in either a cDNA, genomic DNA, or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the terms “complementary” or “complementarity” are used in reference to polynucleotides (i.e., a sequence of nucleotides) related by the base-pairing rules. For example, for the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” refers to a degree of complementarity. There may be partial homology or complete homology (i.e., identity). A partially complementary sequence is one that at least partially inhibits a completely complementary sequence from hybridizing to a target nucleic acid and is referred to using the functional term “substantially homologous.” The term “inhibition of binding,” when used in reference to nucleic acid binding, refers to inhibition of binding caused by competition of homologous sequences for binding to a target sequence. The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that lacks even a partial degree of complementarity (e.g., less than about 30% identity); in the absence of non-specific binding the probe will not hybridize to the second non-complementary target.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.).

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described above.

A gene may produce multiple RNA species that are generated by differential splicing of the primary RNA transcript. cDNAs that are splice variants of the same gene will contain regions of sequence identity or complete homology (representing the presence of the same exon or portion of the same exon on both cDNAs) and regions of complete non-identity (for example, representing the presence of exon “A” on cDNA 1 wherein cDNA 2 contains exon “B” instead). Because the two cDNAs contain regions of sequence identity they will both hybridize to a probe derived from the entire gene or portions of the gene containing sequences found on both cDNAs; the two splice variants are therefore substantially homologous to such a probe and to each other.

When used in reference to a single-stranded nucleic acid sequence, the term “substantially homologous” refers to any probe that can hybridize (i.e., it is the complement of) the single-stranded nucleic acid sequence under conditions of low stringency as described above.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids.

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41(% G+C), when a nucleic acid is in aqueous solution at 1 M NaCl (See e.g., Anderson and Young, Quantitative Filter Hybridization, in Nucleic Acid Hybridization [1985]). Other references include more sophisticated computations that take structural as well as sequence characteristics into account for the calculation of Tₘ.

As used herein the term “stringency” is used in reference to the conditions of temperature, ionic strength, and the presence of other compounds such as organic solvents, under which nucleic acid hybridizations are conducted. Those skilled in the art will recognize that “stringency” conditions may be altered by varying the parameters just described either individually or in concert. With “high stringency” conditions, nucleic acid base pairing will occur only between nucleic acid fragments that have a high frequency of complementary base sequences (e.g., hybridization under “high stringency” conditions may occur between homologs with about 85-100% identity, preferably about 70-100% identity). With medium stringency conditions, nucleic acid base pairing will occur between nucleic acids with an intermediate frequency of complementary base sequences (e.g., hybridization under “medium stringency” conditions may occur between homologs with about 50-70% identity). Thus, conditions of “weak” or “low” stringency are often required with nucleic acids that are derived from organisms that are genetically diverse, as the frequency of complementary sequences is usually less.

“High stringency conditions” when used in reference to nucleic acid hybridization comprise conditions equivalent to binding or hybridization at 42 C in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 0.1×SSPE, 1.0% SDS at 42 C when a probe of about 500 nucleotides in length is employed.

“Medium stringency conditions” when used in reference to nucleic acid hybridization comprise conditions equivalent to binding or hybridization at 42 C in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 1.0×SSPE, 1.0% SDS at 42 C when a probe of about 500 nucleotides in length is employed.

“Low stringency conditions” comprise conditions equivalent to binding or hybridization at 42 C in a solution consisting of 5×SSPE (43.8 g/l NaCl, 6.9 g/l NaH₂PO₄H₂O and 1.85 g/l EDTA, pH adjusted to 7.4 with NaOH), 0.1% SDS, 5× Denhardt's reagent [50× Denhardt's contains per 500 ml: 5 g Ficoll (Type 400, Pharamcia), 5 g BSA (Fraction V; Sigma)] and 100 g/ml denatured salmon sperm DNA followed by washing in a solution comprising 5×SSPE, 0.1% SDS at 42 C when a probe of about 500 nucleotides in length is employed.

The following terms are used to describe the sequence relationships between two or more polynucleotides: “reference sequence,” “sequence identity,” “percentage of sequence identity,” and “substantial identity.” A “reference sequence” is a defined sequence used as a basis for a sequence comparison; a reference sequence may be a subset of a larger sequence, for example, as a segment of a full-length cDNA sequence given in a sequence listing or may comprise a complete gene sequence. Generally, a reference sequence is at least 20 nucleotides in length, frequently at least 25 nucleotides in length, and often at least 50 nucleotides in length. Since two polynucleotides may each (1) comprise a sequence (i.e., a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window,” as used herein, refers to a conceptual segment of at least 20 contiguous nucleotide positions wherein a polynucleotide sequence may be compared to a reference sequence of at least 20 contiguous nucleotides and wherein the portion of the polynucleotide sequence in the comparison window may comprise additions or deletions (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman [Smith and Waterman, Adv. Appl. Math. 2: 482 (1981)] by the homology alignment algorithm of Needleman and Wunsch [Needleman and Wunsch, J. Mol. Biol. 48:443 (1970)], by the search for similarity method of Pearson and Lipman [Pearson and Lipman, Proc. Natl. Acad. Sci. (U.S.A.) 85:2444 (1988)], by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of homology over the comparison window) generated by the various methods is selected. The term “sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity.

As applied to polynucleotides, the term “substantial identity” denotes a characteristic of a polynucleotide sequence, wherein the polynucleotide comprises a sequence that has at least 85 percent sequence identity, preferably at least 90 to 95 percent sequence identity, more usually at least 99 percent sequence identity as compared to a reference sequence over a comparison window of at least 20 nucleotide positions, frequently over a window of at least 25-50 nucleotides, wherein the percentage of sequence identity is calculated by comparing the reference sequence to the polynucleotide sequence which may include deletions or additions which total 20 percent or less of the reference sequence over the window of comparison. The reference sequence may be a subset of a larger sequence, for example, as a splice variant of the full-length sequences.

As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity or more (e.g., 99 percent sequence identity). Preferably, residue positions that are not identical differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine.

“Amplification” is a special case of nucleic acid replication involving template specificity. It is to be contrasted with non-specific template replication (i.e., replication that is template-dependent but not dependent on a specific template). Template specificity is here distinguished from fidelity of replication (i.e., synthesis of the proper polynucleotide sequence) and nucleotide (ribo- or deoxyribo-) specificity. Template specificity is frequently described in terms of “target” specificity. Target sequences are “targets” in the sense that they are sought to be sorted out from other nucleic acid. Amplification techniques have been designed primarily for this sorting out.

Template specificity is achieved in most amplification techniques by the choice of enzyme. Amplification enzymes are enzymes that, under conditions they are used, will process only specific sequences of nucleic acid in a heterogeneous mixture of nucleic acid. For example, in the case of Q replicase, MDV-1 RNA is the specific template for the replicase (D. L. Kacian et al., Proc. Natl. Acad. Sci. USA 69:3038 [1972]). Other nucleic acid will not be replicated by this amplification enzyme. Similarly, in the case of T7 RNA polymerase, this amplification enzyme has a stringent specificity for its own promoters (M. Chamberlin et al., Nature 228:227 [1970]). In the case of T4 DNA ligase, the enzyme will not ligate the two oligonucleotides or polynucleotides, where there is a mismatch between the oligonucleotide or polynucleotide substrate and the template at the ligation junction (D. Y. Wu and R. B. Wallace, Genomics 4:560 [1989]). Finally, Taq and Pfu polymerases, by virtue of their ability to function at high temperature, are found to display high specificity for the sequences bounded and thus defined by the primers; the high temperature results in thermodynamic conditions that favor primer hybridization with the target sequences and not hybridization with non-target sequences (H. A. Erlich (ed.), PCR Technology, Stockton Press [1989]).

As used herein, the term “amplifiable nucleic acid” is used in reference to nucleic acids that may be amplified by any amplification method. It is contemplated that “amplifiable nucleic acid” will usually comprise “sample template.”

As used herein, the term “sample template” refers to nucleic acid originating from a sample that is analyzed for the presence of “target” (defined below). In contrast, “background template” is used in reference to nucleic acid other than sample template that may or may not be present in a sample. Background template is most often inadvertent. It may be the result of carryover, or it may be due to the presence of nucleic acid contaminants sought to be purified away from the sample. For example, nucleic acids from organisms other than those to be detected may be present as background in a test sample.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer should be sufficiently long to prime the synthesis of extension products in the presence of the inducing agent. The exact lengths of the primers will depend on many factors, including temperature, source of primer and the use of the method.

As used herein, the term “probe” or “hybridization probe” refers to an oligonucleotide (i.e., a sequence of nucleotides), whether occurring naturally as in a purified restriction digest or produced synthetically, recombinantly or by PCR amplification, that is capable of hybridizing, at least in part, to another oligonucleotide of interest. A probe may be single-stranded or double-stranded. Probes are useful in the detection, identification and isolation of particular sequences. In some preferred embodiments, probes used in the present invention will be labeled with a “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

As used herein, the term “target” refers to a nucleic acid sequence or structure to be detected or characterized.

As used herein, the term “polymerase chain reaction” (“PCR”) refers to the method of K. B. Mullis (See e.g., U.S. Pat. Nos. 4,683,195, 4,683,202, and 4,965,188, hereby incorporated by reference), which describes a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing, and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified.”

With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of ³²P-labeled deoxynucleotide triphosphates, such as dCTP or dATP, into the amplified segment). In addition to genomic DNA, any oligonucleotide or polynucleotide sequence can be amplified with the appropriate set of primer molecules. In particular, the amplified segments created by the PCR process itself are, themselves, efficient templates for subsequent PCR amplifications.

As used herein, the terms “PCR product,” “PCR fragment,” and “amplification product” refer to the resultant mixture of compounds after two or more cycles of the PCR steps of denaturation, annealing and extension are complete. These terms encompass the case where there has been amplification of one or more segments of one or more target sequences.

As used herein, the term “amplification reagents” refers to those reagents (deoxyribonucleotide triphosphates, buffer, etc.), needed for amplification except for primers, nucleic acid template, and the amplification enzyme. Typically, amplification reagents along with other reaction components are placed and contained in a reaction vessel (test tube, microwell, etc.).

As used herein, the term “reaction vessel” refers to a system in which a reaction may be conducted, including but not limited to test tubes, wells, microwells (e.g., wells in microtitre assay plates such as, 96-well, 384-well and 1536-well assay plates), capillary tubes, ends of fibers such as optical fibers, microfluidic devices such as fluidic chips, cartridges and cards (including but not limited to those described, e.g., in U.S. Pat. No. 6,126,899, to Woudenberg, et al., U.S. Pat. Nos. 6,627,159, 6,720,187, and 6,734,401 to Bedingham, et al., U.S. Pat. Nos. 6,319,469 and 6,709,869 to Mian, et al., U.S. Pat. Nos. 5,587,128 and 6,660,517 to Wilding, et al.), or a test site on any surface (including but not limited to a glass, plastic or silicon surface, a bead, a microchip, or an non-solid surface, such as a gel or a dendrimer).

As used herein, the term “recombinant DNA molecule” as used herein refers to a DNA molecule that is comprised of segments of DNA joined together by means of molecular biological techniques.

As used herein, the term “antisense” is used in reference to RNA sequences that are complementary to a specific RNA sequence (e.g., mRNA). The term “antisense strand” is used in reference to a nucleic acid strand that is complementary to the “sense” strand. The designation (−) (i.e., “negative”) is sometimes used in reference to the antisense strand, with the designation (+) sometimes used in reference to the sense (i.e., “positive”) strand.

The term “isolated” when used in relation to a nucleic acid, as in “an isolated oligonucleotide” or “isolated polynucleotide” refers to a nucleic acid sequence that is identified and separated from at least one contaminant nucleic acid with which it is ordinarily associated in its natural source. Isolated nucleic acid is present in a form or setting that is different from that in which it is found in nature. In contrast, non-isolated nucleic acids are nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acids encoding a polypeptide include, by way of example, such nucleic acid in cells ordinarily expressing the polypeptide where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may single-stranded), but may contain both the sense and anti-sense strands (i.e., the oligonucleotide or polynucleotide may be double-stranded).

As used herein the term “portion” when in reference to a nucleotide sequence (as in “a portion of a given nucleotide sequence”) refers to fragments of that sequence. The fragments may range in size from four nucleotides to the entire nucleotide sequence minus one nucleotide (e.g., 10 nucleotides, 11, . . . , 20, . . . ).

As used herein, the term “purified” or “to purify” refers to the removal of contaminants from a sample. As used herein, the term “purified” refers to molecules (e.g., nucleic or amino acid sequences) that are removed from their natural environment, isolated or separated. An “isolated nucleic acid sequence” is therefore a purified nucleic acid sequence. “Substantially purified” molecules are at least 60% free, preferably at least 75% free, and more preferably at least 90% free from other components with which they are naturally associated.

The term “recombinant protein” or “recombinant polypeptide” as used herein refers to a protein molecule that is expressed from a recombinant DNA molecule.

The term “native protein” as used herein to indicate that a protein does not contain amino acid residues encoded by vector sequences; that is the native protein contains only those amino acids found in the protein as it occurs in nature. A native protein may be produced by recombinant means or may be isolated from a naturally occurring source.

As used herein the term “portion” when in reference to a protein (as in “a portion of a given protein”) refers to fragments of that protein. The fragments may range in size from four consecutive amino acid residues to the entire amino acid sequence minus one amino acid.

The term “Southern blot,” refers to the analysis of DNA on agarose or acrylamide gels to fractionate the DNA according to size followed by transfer of the DNA from the gel to a solid support, such as nitrocellulose or a nylon membrane. The immobilized DNA is then probed with a labeled probe to detect DNA species complementary to the probe used. The DNA may be cleaved with restriction enzymes prior to electrophoresis. Following electrophoresis, the DNA may be partially depurinated and denatured prior to or during transfer to the solid support. Southern blots are a standard tool of molecular biologists (J. Sambrook et al., Molecular Cloning: A Laboratory Manual, Cold Spring Harbor Press, NY, pp 9.31-9.58 [1989]).

The term “Western blot” refers to the analysis of protein(s) (or polypeptides) immobilized onto a support such as nitrocellulose or a membrane. The proteins are run on acrylamide gels to separate the proteins, followed by transfer of the protein from the gel to a solid support, such as nitrocellulose or a nylon membrane. The immobilized proteins are then exposed to antibodies with reactivity against an antigen of interest. The binding of the antibodies may be detected by various methods, including the use of labeled antibodies.

The term “test compound” refers to any chemical entity, pharmaceutical, drug, and the like that are tested in an assay (e.g., a drug screening assay) for any desired activity (e.g., including but not limited to, the ability to treat or prevent a disease, illness, sickness, or disorder of bodily function, or otherwise alter the physiological or cellular status of a sample). Test compounds comprise both known and potential therapeutic compounds. A test compound can be determined to be therapeutic by screening using the screening methods of the present invention. A “known therapeutic compound” refers to a therapeutic compound that has been shown (e.g., through animal trials or prior experience with administration to humans) to be effective in such treatment or prevention.

The term “sample” as used herein is used in its broadest sense. A sample suspected of containing a human chromosome or sequences associated with a human chromosome may comprise a cell, chromosomes isolated from a cell (e.g., a spread of metaphase chromosomes), genomic DNA (in solution or bound to a solid support such as for Southern blot analysis), RNA (in solution or bound to a solid support such as for Northern blot analysis), cDNA (in solution or bound to a solid support) and the like. A sample suspected of containing a protein may comprise a cell, a portion of a tissue, an extract containing one or more proteins and the like. Samples include, but are not limited to, tissue sections, blood, blood fractions (e.g. serum, plasma, cells) saliva, cerebral spinal fluid, pleural fluid, milk, lymph, sputum, semen, urine, feces, aminotic fluid, chorionic villus samples (CVS), cervical swabs and buccal swabs.

The term “label” as used herein refers to any atom or molecule that can be used to provide a detectable (preferably quantifiable) effect, and that can be attached to a nucleic acid or protein. Labels include but are not limited to dyes; radiolabels such as ³²P; binding moieties such as biotin; haptens such as digoxgenin; luminogenic, phosphorescent or fluorogenic moieties; and fluorescent dyes alone or in combination with moieties that can suppress or shift emission spectra by fluorescence resonance energy transfer (FRET). Labels may provide signals detectable by fluorescence, radioactivity, colorimetry, gravimetry, X-ray diffraction or absorption, magnetism, enzymatic activity, and the like. A label may be a charged moiety (positive or negative charge) or alternatively, may be charge neutral. Labels can include or consist of nucleic acid or protein sequence, so long as the sequence comprising the label is detectable.

The term “signal” as used herein refers to any detectable effect, such as would be caused or provided by a label or an assay reaction.

As used herein, the term “detector” refers to a system or component of a system, e.g., an instrument (e.g. a camera, fluorimeter, charge-coupled device, scintillation counter, etc) or a reactive medium (X-ray or camera film, pH indicator, etc.), that can convey to a user or to another component of a system (e.g., a computer or controller) the presence of a signal or effect. A detector can be a photometric or spectrophotometric system, which can detect ultraviolet, visible or infrared light, including fluorescence or chemiluminescence; a radiation detection system; a spectroscopic system such as nuclear magnetic resonance spectroscopy, mass spectrometry or surface enhanced Raman spectrometry; a system such as gel or capillary electrophoresis or gel exclusion chromatography; or other detection system known in the art, or combinations thereof.

As used herein, the term “distribution system” refers to systems capable of transferring and/or delivering materials from one entity to another or one location to another. For example, a distribution system for transferring detection panels from a manufacturer or distributor to a user may comprise, but is not limited to, a packaging department, a mail room, and a mail delivery system. Alternately, the distribution system may comprise, but is not limited to, one or more delivery vehicles and associated delivery personnel, a display stand, and a distribution center. In some embodiments of the present invention interested parties (e.g., detection panel manufactures) utilize a distribution system to transfer detection panels to users at no cost, at a subsidized cost, or at a reduced cost.

As used herein, the term “at a reduced cost” refers to the transfer of goods or services at a reduced direct cost to the recipient (e.g. user). In some embodiments, “at a reduced cost” refers to transfer of goods or services at no cost to the recipient.

As used herein, the term “at a subsidized cost” refers to the transfer of goods or services, wherein at least a portion of the recipient's cost is deferred or paid by another party. In some embodiments, “at a subsidized cost” refers to transfer of goods or services at no cost to the recipient.

As used herein, the term “at no cost” refers to the transfer of goods or services with no direct financial expense to the recipient. For example, when detection panels are provided by a manufacturer or distributor to a user (e.g. research scientist) at no cost, the user does not directly pay for the tests.

The term “detection” as used herein refers to quantitatively or qualitatively identifying an analyte (e.g., DNA, RNA or a protein) within a sample. The term “detection assay” as used herein refers to a kit, test, or procedure performed for the purpose of detecting an analyte nucleic acid within a sample. Detection assays produce a detectable signal or effect when performed in the presence of the target analyte, and include but are not limited to assays incorporating the processes of hybridization, nucleic acid cleavage (e.g., exo- or endonuclease), nucleic acid amplification, nucleotide sequencing, primer extension, or nucleic acid ligation.

As used herein, the term “functional detection oligonucleotide” refers to an oligonucleotide that is used as a component of a detection assay, wherein the detection assay is capable of successfully detecting (i.e., producing a detectable signal) an intended target nucleic acid when the functional detection oligonucleotide provides the oligonucleotide component of the detection assay. This is in contrast to a non-functional detection oligonucleotides, which fail to produce a detectable signal in a detection assay for the particular target nucleic acid when the non-functional detection oligonucleotide is provided as the oligonucleotide component of the detection assay. Determining if an oligonucleotide is a functional oligonucleotide can be carried out experimentally by testing the oligonucleotide in the presence of the particular target nucleic acid using the detection assay.

As used herein, the term “derived from a different subject,” such as samples or nucleic acids derived from a different subjects refers to a samples derived from multiple different individuals. For example, a blood sample comprising genomic DNA from a first person and a blood sample comprising genomic DNA from a second person are considered blood samples and genomic DNA samples that are derived from different subjects. A sample comprising five target nucleic acids derived from different subjects is a sample that includes at least five samples from five different individuals. However, the sample may further contain multiple samples from a given individual.

As used herein, the term “treating together”, when used in reference to experiments or assays, refers to conducting experiments concurrently or sequentially, wherein the results of the experiments are produced, collected, or analyzed together (i.e., during the same time period). For example, a plurality of different target sequences located in separate wells of a multiwell plate or in different portions of a microarray are treated together in a detection assay where detection reactions are carried out on the samples simultaneously or sequentially and where the data collected from the assays is analyzed together.

The terms “assay data” and “test result data” as used herein refer to data collected from performance of an assay (e.g., to detect or quantitate a gene, SNP or an RNA). Test result data may be in any form, i.e., it may be raw assay data or analyzed assay data (e.g., previously analyzed by a different process). Collected data that has not been further processed or analyzed is referred to herein as “raw” assay data (e.g., a number corresponding to a measurement of signal, such as a fluorescence signal from a spot on a chip or a reaction vessel, or a number corresponding to measurement of a peak, such as peak height or area, as from, for example, a mass spectrometer, HPLC or capillary separation device), while assay data that has been processed through a further step or analysis (e.g., normalized, compared, or otherwise processed by a calculation) is referred to as “analyzed assay data” or “output assay data”.

As used herein, the term “database” refers to collections of information (e.g., data) arranged for ease of retrieval, for example, stored in a computer memory. A “genomic information database” is a database comprising genomic information, including, but not limited to, polymorphism information (i.e., information pertaining to genetic polymorphisms), genome information (i.e., genomic information), linkage information (i.e., information pertaining to the physical location of a nucleic acid sequence with respect to another nucleic acid sequence, e.g., in a chromosome), and disease association information (i.e., information correlating the presence of or susceptibility to a disease to a physical trait of a subject, e.g., an allele of a subject). “Database information” refers to information to be sent to a databases, stored in a database, processed in a database, or retrieved from a database. “Sequence database information” refers to database information pertaining to nucleic acid sequences. As used herein, the term “distinct sequence databases” refers to two or more databases that contain different information than one another. For example, the dbSNP and GenBank databases are distinct sequence databases because each contains information not found in the other.

As used herein the terms “processor” and “central processing unit” or “CPU” are used interchangeably and refer to a device that is able to read a program from a computer memory (e.g., ROM or other computer memory) and perform a set of steps according to the program.

As used herein, the terms “computer memory” and “computer memory device” refer to any storage media readable by a computer processor. Examples of computer memory include, but are not limited to, RAM, ROM, computer chips, digital video disc (DVDs), compact discs (CDs), hard disk drives (HDD), and magnetic tape.

As used herein, the term “computer readable medium” refers to any device or system for storing and providing information (e.g., data and instructions) to a computer processor. Examples of computer readable media include, but are not limited to, DVDs, CDs, hard disk drives, magnetic tape and servers for streaming media over networks.

As used herein, the term “hyperlink” refers to a navigational link from one document to another, or from one portion (or component) of a document to another. Typically, a hyperlink is displayed as a highlighted word or phrase that can be selected by clicking on it using a mouse to jump to the associated document or documented portion.

As used herein, the term “hypertext system” refers to a computer-based informational system in which documents (and possibly other types of data entities) are linked together via hyperlinks to form a user-navigable “web.”

As used herein, the term “Internet” refers to any collection of networks using standard protocols. For example, the term includes a collection of interconnected (public and/or private) networks that are linked together by a set of standard protocols (such as TCP/IP, HTTP, and FTP) to form a global, distributed network. While this term is intended to refer to what is now commonly known as the Internet, it is also intended to encompass variations that may be made in the future, including changes and additions to existing standard protocols or integration with other media (e.g., television, radio, etc). The term is also intended to encompass non-public networks such as private (e.g., corporate) Intranets.

As used herein, the terms “World Wide Web” or “web” refer generally to both (i) a distributed collection of interlinked, user-viewable hypertext documents (commonly referred to as Web documents or Web pages) that are accessible via the Internet, and (ii) the client and server software components which provide user access to such documents using standardized Internet protocols. Currently, the primary standard protocol for allowing applications to locate and acquire Web documents is HTTP, and the Web pages are encoded using HTML. However, the terms “Web” and “World Wide Web” are intended to encompass future markup languages and transport protocols that may be used in place of (or in addition to) HTML and HTTP.

As used herein, the term “web site” refers to a computer system that serves informational content over a network using the standard protocols of the World Wide Web. Typically, a Web site corresponds to a particular Internet domain name and includes the content associated with a particular organization. As used herein, the term is generally intended to encompass both (i) the hardware/software server components that serve the informational content over the network, and (ii) the “back end” hardware/software components, including any non-standard or specialized components, that interact with the server components to perform services for Web site users.

As used herein, the term “HTML” refers to HyperText Markup Language that is a standard coding convention and set of codes for attaching presentation and linking attributes to informational content within documents. HTML is based on SGML, the Standard Generalized Markup Language. During a document authoring stage, the HTML codes (referred to as “tags”) are embedded within the informational content of the document. When the Web document (or HTML document) is subsequently transferred from a Web server to a browser, the codes are interpreted by the browser and used to parse and display the document. Additionally, in specifying how the Web browser is to display the document, HTML tags can be used to create links to other Web documents (commonly referred to as “hyperlinks”).

As used herein, the term “XML” refers to Extensible Markup Language, an application profile that, like HTML, is based on SGML. XML differs from HTML in that: information providers can define new tag and attribute names at will; document structures can be nested to any level of complexity; any XML document can contain an optional description of its grammar for use by applications that need to perform structural validation. XML documents are made up of storage units called entities, which contain either parsed or unparsed data. Parsed data is made up of characters, some of which form character data, and some of which form markup. Markup encodes a description of the document's storage layout and logical structure. XML provides a mechanism to impose constraints on the storage layout and logical structure, to define constraints on the logical structure and to support the use of predefined storage units. A software module called an XML processor is used to read XML documents and provide access to their content and structure.

As used herein, the term “HTTP” refers to HyperText Transport Protocol that is the standard World Wide Web client-server protocol used for the exchange of information (such as HTML documents, and client requests for such documents) between a browser and a Web server. HTTP includes a number of different types of messages that can be sent from the client to the server to request different types of server actions. For example, a “GET” message, which has the format GET, causes the server to return the document or file located at the specified URL.

As used herein, the term “URL” refers to Uniform Resource Locator that is a unique address that fully specifies the location of a file or other resource on the Internet. The general format of a URL is protocol://machine address:port/path/filename. The port specification is optional, and if none is entered by the user, the browser defaults to the standard port for whatever service is specified as the protocol. For example, if HTTP is specified as the protocol, the browser will use the HTTP default port of 80.

As used herein, the term “PUSH technology” refers to an information dissemination technology used to send data to users over a network. In contrast to the World Wide Web (a “pull” technology), in which the client browser should request a Web page before it is sent, PUSH protocols send the informational content to the user computer automatically, typically based on information pre-specified by the user.

As used herein, the term “communication network” refers to any network that allows information to be transmitted from one location to another. For example, a communication network for the transfer of information from one computer to another includes any public or private network that transfers information using electrical, optical, satellite transmission, and the like. Two or more devices that are part of a communication network such that they can directly or indirectly transmit information from one to the other are considered to be “in electronic communication” with one another. A computer network containing multiple computers may have a central computer (“central node”) that processes information to one or more sub-computers that carry out specific tasks (“sub-nodes”). Some networks comprises computers that are in “different geographic locations” from one another, meaning that the computers are located in different physical locations (i.e., aren't physically the same computer, e.g., are located in different countries, states, cities, rooms, etc.).

As used herein, the term “detection assay component” refers to a component of a system capable of performing a detection assay. Detection assay components include, but are not limited to, hybridization probes, buffers, and the like.

As used herein, the term “a detection assays configured for target detection” refers to a collection of assay components that are capable of producing a detectable signal when carried out using the target nucleic acid. For example, a detection assay that has empirically been demonstrated to detect a particular single nucleotide polymorphism is considered a detection assay configured for target detection.

As used herein, the phrase “unique detection assay” refers to a detection assay that has a different collection of detection assay components in relation to other detection assays located on the same detection panel. A unique assay doesn't necessarily detect a different target (e.g. SNP) than other assays on the same detection panel, but it does have a least one difference in the collection of components used to detect a given target (e.g. a unique detection assay may employ a probe sequences that is shorter or longer in length than other assays on the same detection panel).

As used herein, the term “candidate” refers to an assay or analyte, e.g., a nucleic acid, suspected of having a particular feature or property. A “candidate sequence” refers to a nucleic acid suspected of comprising a particular sequence, while a “candidate oligonucleotide” refers to an oligonucleotide suspected of having a property such as comprising a particular sequence, or having the capability to hybridize to a target nucleic acid or to perform in a detection assay. A “candidate detection assay” refers to a detection assay that is suspected of being a valid detection assay.

As used herein, the term “detection panel” refers to a substrate or device containing at least two unique candidate detection assays configured for target detection.

As used herein, the term “valid detection assay” refers to a detection assay that has been shown to accurately predict an association between the detection of a target and a phenotype (e.g. medical condition). Examples of valid detection assays include, but are not limited to, detection assays that, when a target is detected, accurately predict the phenotype medical 95%, 96%, 97%, 98%, 99%, 99.5%, 99.8%, or 99.9% of the time. Other examples of valid detection assays include, but are not limited to, detection assays that quality as and/or are marketed as Analyte-Specific Reagents (i.e. as defined by FDA regulations) or In-Vitro Diagnostics (i.e. approved by the FDA).

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. As used herein, the term “fragmented kit” refers to a delivery systems comprising two or more separate containers that each contain a subportion of the total kit components. The containers may be delivered to the intended recipient together or separately. For example, a first container may contain an enzyme for use in an assay, while a second container contains oligonucleotides. The term “fragmented kit” is intended to encompass kits containing Analyte specific reagents (ASR's) regulated under section 520(e) of the Federal Food, Drug, and Cosmetic Act, but are not limited thereto. Indeed, any delivery system comprising two or more separate containers that each contains a subportion of the total kit components are included in the term “fragmented kit.” In contrast, a “combined kit” refers to a delivery system containing all of the components of a reaction assay in a single container (e.g., in a single box housing each of the desired components). The term “kit” includes both fragmented and combined kits.

As used herein, the term “information” refers to any collection of facts or data. In reference to information stored or processed using a computer system(s), including but not limited to internets, the term refers to any data stored in any format (e.g., analog, digital, optical, etc.). As used herein, the term “information related to a subject” refers to facts or data pertaining to a subject (e.g., a human, plant, or animal). The term “genomic information” refers to information pertaining to a genome including, but not limited to, nucleic acid sequences, genes, allele frequencies, RNA expression levels, protein expression, phenotypes correlating to genotypes, etc. “Allele frequency information” refers to facts or data pertaining allele frequencies, including, but not limited to, allele identities, statistical correlations between the presence of an allele and a characteristic of a subject (e.g., a human subject), the presence or absence of an allele in a individual or population, the percentage likelihood of an allele being present in an individual having one or more particular characteristics, etc.

As used herein, the term “assay validation information” refers to genomic information and/or allele frequency information resulting from processing of test result data (e.g. processing with the aid of a computer). Assay validation information may be used, for example, to identify a particular candidate detection assay as a valid detection assay.

### DETAILED DESCRIPTION OF THE INVENTION

Detection in Biological Samples

A goal in molecular diagnostics has been to achieve accurate, sensitive detection of analytes in as little time as possible with the least amount of labor and steps as possible. One manner in which this is achieved is the multiplex detection of analytes in samples, allowing multiple detection events in a single reaction vessel or solution. However, many of the existing diagnostic methods, including multiplex reaction, still require many steps, including sample preparation steps that add to the time, complexity, and cost of conducting reactions. The present invention, in some embodiments, provides solutions to these problems by providing assay that can be conducted directly in unpurified or untreated biological samples (e.g., blood).

Direct detection in biological samples (e.g., blood, saliva, urine, etc.) has been elusive because of the presence of numerous biological factors in natural samples that can interfere with the function, accuracy, and consistency of diagnostic reactions. For example, many nucleic acid detection technologies employ enzymes or other reagents that are sensitive to specific salt and pH conditions or that are subject to proteolysis or inhibition by natural factors. The present invention provides systems and methods for use of the INVADER assay, alone or in combination with PCR or related technologies, for the direct detection of nucleic acid target sequences in unpurified bodily fluids. Example 12 below provides one such example. Such methods may be employed as individual reactions or may be employed as multiplex reactions. Several multiplex embodiments are described in detail below.

Thus, in some embodiments, the present invention provides systems, compositions, kits, and methods for detecting one or more target nucleic acids in unpurified (or partially purified) bodily fluids comprising the step of exposing an unpurified bodily fluid to detection assay reagents under conditions such that the target nucleic acid is detected, if present. In preferred embodiments, the method is carried out in a single step reaction. For example, once the sample is exposed to the reagents, there is not need to add additional reagents prior to the detection step. Thus, the method can be carried out in a reaction vessel (e.g., a closed reaction vessel) without the need for addition human or other intervention. In preferred embodiments, the method involves an invasive cleavage reaction with or without the polymerase chain reaction. Because of the signal amplification, sensitivity, and ability to quantitate signal using an invasive cleavage reaction, where the polymerase chain reaction is used, limited cycles need only be used (e.g., 20, 15, 12, 10, or fewer). The kits for conducting or assisting in such methods may comprise any one or more of the reagents useful in the methods. For example, in some embodiments, the kits comprise a polymerase, a 5′ nuclease (e.g., a FEN-1 endonuclease), and a buffer that permits detectable amplification of the target nucleic acid in an unpurified bodily fluid.

Multiplex Reactions

Since its introduction in 1988 (Chamberlain, et al. Nucleic Acids Res., 16:11141 (1988)), multiplex PCR has become a routine means of amplifying multiple genetic loci in a single reaction. This approach has found utility in a number of research, as well as clinical, applications. Multiplex PCR has been described for use in diagnostic virology (Elnifro, et al. Clinical Microbiology Reviews, 13: 559 (2000)), paternity testing (Hidding and Schmitt, Forensic Sci. Int., 113: 47 (2000); Bauer et al., Int. J. Legal Med. 116: 39 (2002)), preimplantation genetic diagnosis (Ouhibi, et al., Curr Womens Health Rep. 1: 138 (2001)), microbial analysis in environmental and food samples (Rudi et al., Int J Food Microbiology, 78: 171 (2002)), and veterinary medicine (Zarlenga and Higgins, Vet Parasitol. 101: 215 (2001)), among others. Most recently, expansion of genetic analysis to whole genome levels, particularly for single nucleotide polymorphisms, or SNPs, has created a need highly multiplexed PCR capabilities. Comparative genome-wide association and candidate gene studies require the ability to genotype between 100,000-500,000 SNPs per individual (Kwok, Molecular Medicine Today, 5: 538-5435 (1999); Kwok, Pharmacogenomics, 1: 231 (2000); Risch and Merikangas, Science, 273: 1516 (1996)). Moreover, SNPs in coding or regulatory regions alter gene function in important ways (Cargill et al. Nature Genetics, 22: 231 (1999); Halushka et al., Nature Genetics, 22: 239 (1999)), making these SNPs useful diagnostic tools in personalized medicine (Hagmann, Science, 285: 21 (1999); Cargill et al. Nature Genetics, 22: 231 (1999); Halushka et al., Nature Genetics, 22: 239 (1999)). Likewise, validating the medical association of a set of SNPs previously identified for their potential clinical relevance as part of a diagnostic panel will mean testing thousands of individuals for thousands of markers at a time.

Despite its broad appeal and utility, several factors complicate multiplex PCR amplification. Chief among these is the phenomenon of PCR or amplification bias, in which certain loci are amplified to a greater extent than others. Two classes of amplification bias have been described. One, referred to as PCR drift, is ascribed to stochastic variation in such steps as primer annealing during the early stages of the reaction (Polz and Cavanaugh, Applied and Environmental Microbiology, 64: 3724 (1998)), is not reproducible, and may be more prevalent when very small amounts of target molecules are being amplified (Walsh et al., PCR Methods and Applications, 1: 241 (1992)). The other, referred to as PCR selection, pertains to the preferential amplification of some loci based on primer characteristics, amplicon length, G-C content, and other properties of the genome (Polz, supra).

Another factor affecting the extent to which PCR reactions can be multiplexed is the inherent tendency of PCR reactions to reach a plateau phase. The plateau phase is seen in later PCR cycles and reflects the observation that amplicon generation moves from exponential to pseudo-linear accumulation and then eventually stops increasing. This effect appears to be due to non-specific interactions between the DNA polymerase and the double stranded products themselves. The molar ratio of product to enzyme in the plateau phase is typically consistent for several DNA polymerases, even when different amounts of enzyme are included in the reaction, and is approximately 30:1 product:enzyme. This effect thus limits the total amount of double-stranded product that can be generated in a PCR reaction such that the number of different loci amplified must be balanced against the total amount of each amplicon desired for subsequent analysis, e.g. by gel electrophoresis, primer extension, etc.

Because of these and other considerations, although multiplexed PCR including 50 loci has been reported (Lindblad-Toh et al., Nature Genet. 4: 381 (2000)), multiplexing is typically limited to fewer than ten distinct products. However, given the need to analyze as many as 100,000 to 450,000 SNPs from a single genomic DNA sample there is a clear need for a means of expanding the multiplexing capabilities of PCR reactions.

The present invention provides methods for substantial multiplexing of PCR reactions by, for example, combining the INVADER assay with multiplex PCR amplification. The INVADER assay provides a detection step and signal amplification that allows very large numbers of targets to be detected in a multiplex reaction. As desired, hundreds to thousands to hundreds of thousands of targets may be detected in a multiplex reaction.

Direct genotyping by the INVADER assay typically uses from 5 to 100 ng of human genomic DNA per SNP, depending on detection platform. For a small number of assays, the reactions can be performed directly with genomic DNA without target pre-amplification, however, with more than 100,000 INVADER assays being developed and even larger number expected for genome-wide association studies, the amount of sample DNA may become a limiting factor.

Because the INVADER assay provides from 10⁶ to 10⁷ fold amplification of signal, multiplexed PCR in combination with the INVADER assay would use only limited target amplification as compared to a typical PCR. Consequently, low target amplification level alleviates interference between individual reactions in the mixture and reduces the inhibition of PCR by it's the accumulation of its products, thus providing for more extensive multiplexing. Additionally, it is contemplated that low amplification levels decrease a probability of target cross-contamination and decrease the number of PCR-induced mutations.

Uneven amplification of different loci presents one of biggest challenges in the development of multiplexed PCR. Difference in amplification factors between two loci may result in a situation where the signal generated by an INVADER reaction with a slow-amplifying locus is below the limit of detection of the assay, while the signal from a fast-amplifying locus is beyond the saturation level of the assay. This problem can be addressed in several ways. In some embodiments, the INVADER reactions can be read at different time points, e.g., in real-time, thus significantly extending the dynamic range of the detection. In other embodiments, multiplex PCR can be performed under conditions that allow different loci to reach more similar levels of amplification. For example, primer concentrations can be limited, thereby allowing each locus to reach a more uniform level of amplification. In yet other embodiments, concentrations of PCR primers can be adjusted to balance amplification factors of different loci.

The present invention provides for the design and characteristics of highly multiplex PCR including hundreds to thousands of products in a single reaction. For example, the target pre-amplification provided by hundred-plex PCR reduces the amount of human genomic DNA required for INVADER-based SNP genotyping to less than 0.1 ng per assay. The specifics of highly multiplex PCR optimization and a computer program for the primer design are described below.

In addition to providing methods for highly multiplex PCR, the present invention further provides methods of conducting target and signal amplification reactions in a single reaction vessel with no subsequent manipulations or reagent additions beyond initial reaction set-up. Such combined reactions are suitable for quantitative analysis of limiting target quantities in very short reaction times.

The following discussion provides a description of certain preferred illustrative embodiments of the present invention and is not intended to limit the scope of the present invention.

I. Multiplex PCR Primer Design

The INVADER assay can be used for the detection of single nucleotide polymorphisms (SNPs) with as little as 100-10 ng of genomic DNA without the need for target pre-amplification. However, with more than 50,000 INVADER assays being developed and the potential for whole genome association studies involving hundreds of thousands of SNPs, the amount of sample DNA becomes a limiting factor for large scale analysis. Due to the sensitivity of the INVADER assay on human genomic DNA (hgDNA) without target amplification, multiplex PCR coupled with the INVADER assay requires only limited target amplification (10³-10⁴) as compared to typical multiplex PCR reactions which require extensive amplification (10⁹-10¹²) for conventional gel detection methods. The low level of target amplification used for INVADER™ detection provides for more extensive multiplexing by avoiding amplification inhibition commonly resulting from target accumulation.

The present invention provides methods and selection criteria that allow primer sets for multiplex PCR to be generated (e.g. that can be coupled with a detection assay, such as the INVADER assay). In some embodiments, software applications of the present invention automated multiplex PCR primer selection, thus allowing highly multiplexed PCR with the primers designed thereby. Using the INVADER Medically Associated Panel (MAP) as a corresponding platform for SNP detection, as shown in example 2, the methods, software, and selection criteria of the present invention allowed accurate genotyping of 94 of the 101 possible amplicons (˜93%) from a single PCR reaction. The original PCR reaction used only 10 ng of hgDNA as template, corresponding to less than 150 pg hgDNA per INVADER assay.

The INVADER assay allows for the simultaneous detection of two distinct alleles in the same reaction using an isothermal, single addition format. Allele discrimination takes place by “structure specific” cleavage of the Probe, releasing a 5′ flap which corresponds to a given polymorphism. In the second reaction, the released 5′ flap mediates signal generation by cleavage of the appropriate FRET cassette.

Creation of one of the primer pairs (both a forward and reverse primer) for a 101 primer sets from sequences available for analysis on the INVADER Medically Associated Panel using one embodiment of the software application of the present invention involves sample input file of a single entry (e.g. target sequence information for a single target sequence containing a SNP that is processed the method and software of the present invention). The target sequence information includes Third Wave Technologies's SNP#, short name identifier, and sequence with the SNP location indicated in brackets. Sample output file of a the same entry (e.g. shows the target sequence after being processed by the systems and methods and software of the present invention includes the sequence of the footprint region (capital letters flanking SNP site, showing region where INVADER assay probes hybridize to this target sequence in order to detect the SNP in the target sequence), forward and reverse primer sequences (bold), and their corresponding Tms.

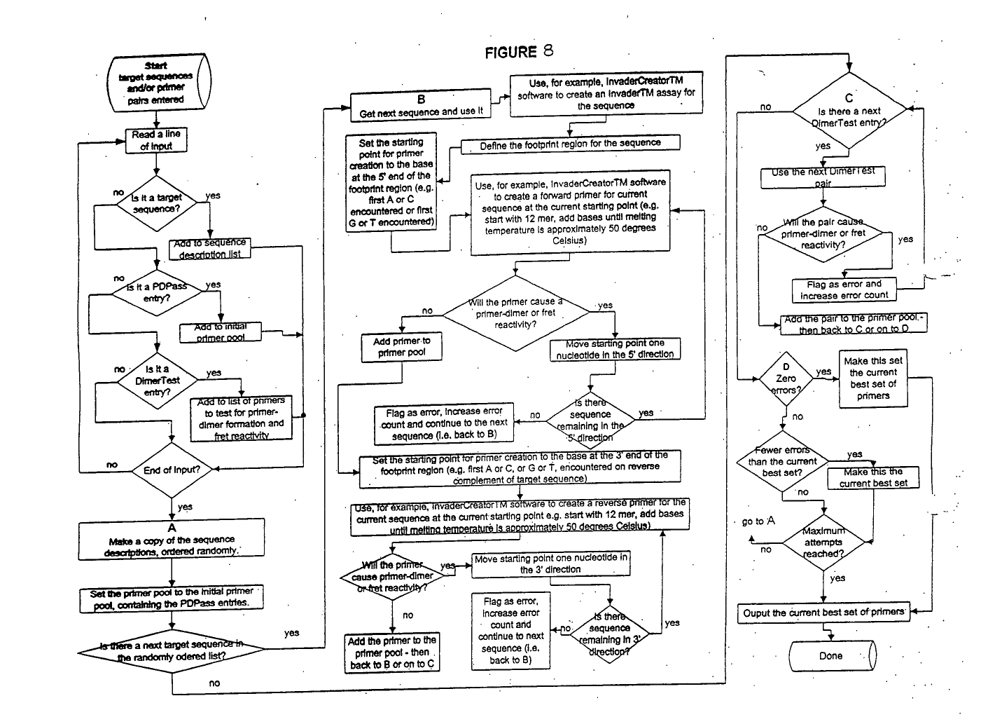

In some embodiments, the selection of primers to make a primer set capable of multiplex PCR is performed in automated fashion (e.g. by a software application). Automated primer selection for multiplex PCR may be accomplished employing a software program designed as shown by the flow chart in FIG. 8.

Multiplex PCR commonly requires extensive optimization to avoid biased amplification of select amplicons and the amplification of spurious products resulting from the formation of primer-dimers. In order to avoid these problems, the present invention provides methods and software application that provide selection criteria to generate a primer set configured for multiplex PCR, and subsequent use in a detection assay (e.g. INVADER detection assays).

In some embodiments, the methods and software applications of the present invention start with user defined sequences and corresponding SNP locations. In certain embodiments, the methods and/or software application determines a footprint region within the target sequence (the minimal amplicon required for INVADER detection) for each sequence. The footprint region includes the region where assay probes hybridize, as well as any user defined additional bases extending outward therefore (e.g. 5 additional bases included on each side of where the assay probes hybridize). Next, primers are designed outward from the footprint region and evaluated against several criteria, including the potential for primer-dimer formation with previously designed primers in the current multiplexing set. This process may be continued through multiple iterations of the same set of sequences until primers against all sequences in the current multiplexing set can be designed.

Once a primer set is designed, multiplex PCR may be carried out, for example, under standard conditions using only 10 ng of hgDNA as template. After 10 min at 95° C., Taq (2.5 units) may be added to a 50 ul reaction and PCR carried out for 50 cycles. The PCR reaction may be diluted and loaded directly onto an INVADER MAP plate (3 ul/well). An additional 3 ul of 15 mM MgCl₂ may be added to each reaction on the INVADER MAP plate and covered with 6 ul of mineral oil. The entire plate may then be heated to 95° C. for 5 min. and incubated at 63° C. for 40 min. FAM and RED fluorescence may then be measured on a Cytofluor 4000 fluorescent plate reader and “Fold Over Zero” (FOZ) values calculated for each amplicon. Results from each SNP may be color coded in a table as “pass” (green), “mis-call” (pink), or “no-call” (white) (See, Example 2 below).

In some embodiments the number of PCR reactions is from about 1 to about 10 reactions. In some embodiments, the number of PCR reactions is from about 10 to about 50 reactions. In further embodiments, the number of PCR reactions is from about 50 to about 100. In additional embodiments, the number of PCR reactions is from about than 100 to 1,000. In still other embodiments, the number of PCR reactions is greater than 1,000.

The present invention also provides methods to optimize multiplex PCR reactions (e.g. once a primer set is generated, the concentration of each primer or primer pair may be optimized). For example, once a primer set has been generated and used in a multiplex PCR at equal molar concentrations, the primers may be evaluated separately such that the optimum primer concentration is determined such that the multiplex primer set performs better.

Multiplex PCR reactions are being recognized in the scientific, research, clinical and biotechnology industries as potentially time effective and less expensive means of obtaining nucleic acid information compared to standard, monoplex PCR reactions. Instead of performing only a single amplification reaction per reaction vessel (tube or well of a multi-well plate for example), numerous amplification reactions are performed in a single reaction vessel.

The cost per target is theoretically lowered by eliminating technician time in assay set-up and data analysis, and by the substantial reagent savings (especially enzyme cost). Another benefit of the multiplex approach is that far less target sample is required. In whole genome association studies involving hundreds of thousands of single nucleotide polymorphisms (SNPs), the amount of target or test sample is limiting for large scale analysis, so the concept of performing a single reaction, using one sample aliquot to obtain, for example, 100 results, versus using 100 sample aliquots to obtain the same data set is an attractive option.

To design primers for a successful multiplex PCR reaction, the issue of aberrant interaction among primers should be addressed. The formation of primer dimers, even if only a few bases in length, may inhibit both primers from correctly hybridizing to the target sequence. Further, if the dimers form at or near the 3′ ends of the primers, no amplification or very low levels of amplification will occur, since the 3′ end is required for the priming event. Clearly, the more primers utilized per multiplex reaction, the more aberrant primer interactions are possible. The methods, systems and applications of the present help prevent primer dimers in large sets of primers, making the set suitable for highly multiplexed PCR.

When designing primer pairs for numerous site (for example 100 sites in a multiplex PCR reaction), the order in which primer pairs are designed can influence the total number of compatible primer pairs for a reaction. For example, if a first set of primers is designed for a first target region that happens to be an A/T rich target region, these primer will be A/T rich. If the second target region chosen also happens to be an A/T rich target region, it is far more likely that the primers designed for these two sets will be incompatible due to aberrant interactions, such as primer dimers. If, however, the second target region chosen is not A/T rich, it is much more likely that a primer set can be designed that will not interact with the first A/T rich set. For any given set of input target sequences, the present invention randomizes the order in which primer sets are designed (See, FIG. 8). Furthermore, in some embodiments, the present invention re-orders the set of input target sequences in a plurality of different, random orders to maximize the number of compatible primer sets for any given multiplex reaction (See, FIG. 8).

The present invention provides criteria for primer design that minimize 3′ interactions while maximizing the number of compatible primer pairs for a given set of reaction targets in a multiplex design. For primers described as 5′-N[x]-N[x−1]- . . . -N[4]-N[3]-N[2]-N[1]-3′, N[1] is an A or C (in alternative embodiments, N[1] is a G or T). N[2]-N[1] of each of the forward and reverse primers designed should not be complementary to N[2]-N[1] of any other oligonucleotide. In certain embodiments, N[3]-N[2]-N[1] should not be complementary to N[3]-N[2]-N[1] of any other oligonucleotide. In preferred embodiments, if these criteria are not met at a given N[I], the next base in the 5′ direction for the forward primer or the next base in the 3′ direction for the reverse primer may be evaluated as an N[1] site. This process is repeated, in conjunction with the target randomization, until all criteria are met for all, or a large majority of, the targets sequences (e.g. 95% of target sequences can have primer pairs made for the primer set that fulfill these criteria).

Another challenge to be overcome in a multiplex primer design is the balance between actual, required nucleotide sequence, sequence length, and the oligonucleotide melting temperature (Tm) constraints. Importantly, since the primers in a multiplex primer set in a reaction should function under the same reaction conditions of buffer, salts and temperature, they need therefore to have substantially similar Tm's, regardless of GC or AT richness of the region of interest. The present invention allows for primer design which meet minimum Tm and maximum Tm requirements and minimum and maximum length requirements. For example, in the formula for each primer 5′-N[x]-N[x−1]- . . . -N[4]-N[3]-N[2]-N[1]-3′, x is selected such the primer has a predetermined melting temperature (e.g. bases are included in the primer until the primer has a calculated melting temperature of about 50 degrees Celsius).

Often the products of a PCR reaction are used as the target material for another nucleic acid detection means, such as a hybridization-type detection assays, or the INVADER reaction assays for example. Consideration should be given to the location of primer placement to allow for the secondary reaction to successfully occur, and again, aberrant interactions between amplification primers and secondary reaction oligonucleotides should be minimized for accurate results and data. Selection criteria may be employed such that the primers designed for a multiplex primer set do not react (e.g. hybridize with, or trigger reactions) with oligonucleotide components of a detection assay. For example, in order to prevent primers from reacting with the FRET oligonucleotide of a bi-plex INVADER assay, certain homology criteria is employed. In particular, if each of the primers in the set are defined as 5′-N[x]-N[x−1]- . . . -N[4]-N[3]-N[2]-N[1]-3′, then N[4]-N[3]-N[2]-N[1]-3′ is selected such that it is less than 90% homologous with the FRET or INVADER oligonucleotides. In other embodiments, N[4]-N[3]-N[2]-N[1]-3′ is selected for each primer such that it is less than 80% homologous with the FRET or INVADER oligonucleotides. In certain embodiments, N[4]-N[3]-N[2]-N[1]-3′ is selected for each primer such that it is less than 70% homologous with the FRET or INVADER oligonucleotides.

While employing the criteria of the present invention to develop a primer set, some primer pairs may not meet all of the stated criteria (these may be rejected as errors). For example, in a set of 100 targets, 30 are designed and meet all listed criteria, however, set 31 fails. In the method of the present invention, set 31 may be flagged as failing, and the method could continue through the list of 100 targets, again flagging those sets which do not meet the criteria (See FIG. 8). Once all 100 targets have had a chance at primer design, the method would note the number of failed sets, re-order the 100 targets in a new random order and repeat the design process (See, FIG. 8). After a configurable number of runs, the set with the most passed primer pairs (the least number of failed sets) are chosen for the multiplex PCR reaction (See FIG. 8).

FIG. 8 shows a flow chart with the basic flow of certain embodiments of the methods and software application of the present invention. In preferred embodiments, the processes detailed in FIG. 8 are incorporated into a software application for ease of use (although, the methods may also be performed manually using, for example, FIG. 8 as a guide).

Target sequences and/or primer pairs are entered into the system shown in FIG. 8. The first set of boxes show how target sequences are added to the list of sequences that have a footprint determined (See “B” in FIG. 8), while other sequences are passed immediately into the primer set pool (e.g. PDPass, those sequences that have been previously processed and shown to work together without forming Primer dimers or having reactivity to FRET sequences), as well as DimerTest entries (e.g. pair or primers a user wants to use, but that has not been tested yet for primer dimer or fret reactivity). In other words, the initial set of boxes leading up to “end of input” sort the sequences so they can be later processed properly.

Starting at “A” in FIG. 8, the primer pool is basically cleared or “emptied” to start a fresh run. The target sequences are then sent to “B” to be processed, and DimerTest pairs are sent to “C” to be processed. Target sequences are sent to “B”, where a user or software application determines the footprint region for the target sequence (e.g. where the assay probes will hybridize in order to detect the mutation (e.g. SNP) in the target sequence). It is important to design this region (which the user may further expand by defining that additional bases past the hybridization region be added) such that the primers that are designed fully encompass this region. In FIG. 8, the software application INVADER CREATOR is used to design the INVADER oligonuclotide and downstream probes that will hybridize with the target region (although any type of program of system could be used to create any type of probes a user was interested in designing probes for, and thus determining the footprint region for on the target sequence). Thus the core footprint region is then defined by the location of these two assay probes on the target.

Next, the system starts from the 5′ edge of the footprint and travels in the 5′ direction until the first base is reached, or until the first A or C (or G or T) is reached. This is set as the initial starting point for defining the sequence of the forward primer (i.e. this serves as the initial N[1] site). From this initial N[1] site, the sequence of the primer for the forward primer is the same as those bases encountered on the target region. For example, if the default size of the primer is set as 12 bases, the system starts with the bases selected as N[1] and then adds the next 11 bases found in the target sequences. This 12-mer primer is then tested for a melting temperature (e.g. using INVADER CREATOR), and additional bases are added from the target sequence until the sequence has a melting temperature that is designated by the user as the default minimum and maximum melting temperatures (e.g. about 50 degrees Celsius, and not more than 55 degrees Celsius). For example, the system employs the formula 5′-N[x]-N[x−1]- . . . -N[4]-N[3]-N[2]-N[1]-3′, and x is initially 12. Then the system adjusts x to a higher number (e.g. longer sequences) until the pre-set melting temperature is found. In certain embodiments, a maximum primer size is employed as a default parameter to serve as an upper limit on the length of the primers designed. In some embodiments, the maximum primer size is about 30 bases (e.g. 29 bases, 30, bases, or 31 bases). On other embodiments, the default settings (e.g. minimum and maximum primer size, and minimum and maximum Tm) are able to be modified using standard database manipulation tools.

The next box in FIG. 8, is used to determine if the primer that has been designed so far will cause primer-dimer and/or fret reactivity (e.g. with the other sequences already in the pool). The criteria used for this determination are explained above. If the primer passes this step, the forward primer is added to the primer pool. However, if the forward primer fails this criteria, as shown in FIG. 8, the starting point (N[1] is moved) one nucleotide in the 5′ direction (or to the next A or C, or next G or T). The system first checks to make sure shifting over leaves enough room on the target sequence to successfully make a primer. If yes, the system loops back and check this new primer for melting temperature. However, if no sequence can be designed, then the target sequence is flagged as an error (e.g. indicating that no forward primer can be made for this target).

This same process is then repeated for designing the reverse primer, as shown in FIG. 8. If a reverse primer is successfully made, then the pair or primers is put into the primer pool, and the system goes back to “B” (if there are more target sequences to process), or goes onto “C” to test DimerTest pairs.

Starting a “C” in FIG. 8 shows how primer pairs that are entered as primers (DimerTest) are processed by the system. If there are no DimerTest pairs, as shown in FIG. 8, the system goes on to “D”. However, if there are DimerTest pairs, these are tested for primer-dimer and/or FRET reactivity as described above. If the DimerTest pair fails these criteria they are flagged as errors. If the DimerTest pair passes the criteria, they are added to the primer set pool, and then the system goes back to “C” if there are more DimerTest pairs to be evaluated, or or goes on to “D” if there are no more DimerTest pairs to be evaluated.

Starting at “D” in FIG. 8, the pool of primers that has been created is evaluated. The first step in this section is to examine the number of error (failures) generated by this particular randomized run of sequences. If there were no errors, this set is the best set as maybe ouputted to a user. If there are more than zero errors, the system compares this run to any other previous runs to see what run resulted in the fewest errors. If the current run has fewer errors, it is designated as the current best set. At this point, the system may go back to “A” to start the run over with another randomized set of the same sequences, or the pre-set maximum number of runs (e.g. 5 runs) may have been reached on this run (e.g. this was the 5th run, and the maximum number of runs was set as 5). If the maximum has been reached, then the best set is outputted as the best set. This best set of primers may then be used to generate as physical set of oligonucleotides such that a multiplex PCR reaction may be carried out.

Another challenge to be overcome with multiplex PCR reactions is the unequal amplicon concentrations that result in a standard multiplex reaction. The different loci targeted for amplification may each behave differently in the amplification reaction, yielding vastly different concentrations of each of the different amplicon products. The present invention provides methods, systems, software applications, computer systems, and a computer data storage medium that may be used to adjust primer concentrations relative to a first detection assay read (e.g. INVADER assay read), and then with balanced primer concentrations come close to substantially equal concentrations of different amplicons.

The concentrations for various primer pairs may be determined experimentally. In some embodiments, there is a first run conducted with all of the primers in equimolar concentrations. Time reads are then conducted. Based upon the time reads, the relative amplification factors for each amplicon are determined. Then based upon a unifying correction equation, an estimate of what the primer concentration should be obtained to get the signals closer within the same time point. These detection assays can be on an array of different sizes (384 well plates).

It is appreciated that combining the invention with detection assays and arrays of detection assays provides substantial processing efficiencies. Employing a balanced mix of primers or primer pairs created using the invention, a single point read can be carried out so that an average user can obtain great efficiencies in conducting tests that require high sensitivity and specificity across an array of different targets.

Having optimized primer pair concentrations in a single reaction vessel allows the user to conduct amplification for a plurality or multiplicity of amplification targets in a single reaction vessel and in a single step. The yield of the single step process is then used to successfully obtain test result data for, for example, several hundred assays. For example, each well on a 384 well plate can have a different detection assay thereon. The results of the single step mutliplex PCR reaction has amplified 384 different targets of genomic DNA, and provides you with 384 test results for each plate. Where each well has a plurality of assays even greater efficiencies can be obtained.

Therefore, the present invention provides the use of the concentration of each primer set in highly multiplexed PCR as a parameter to achieve an unbiased amplification of each PCR product. Any PCR includes primer annealing and primer extension steps. Under standard PCR conditions, high concentration of primers in the order of 1 uM ensures fast kinetics of primers annealing while the optimal time of the primer extension step depends on the size of the amplified product and can be much longer than the annealing step. By reducing primer concentration, the primer annealing kinetics can become a rate limiting step and PCR amplification factor should strongly depend on primer concentration, association rate constant of the primers, and the annealing time.

The binding of primer P with target T can be described by the following model: 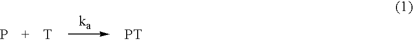
where kₐ is the association rate constant of primer annealing. We assume that the annealing occurs at the temperatures below primer melting and the reverse reaction can be ignored.

The solution for this kinetics under the conditions of a primer excess is well known:
[PT]=T₀(1−e^{-k}^{ₐ}^{ct}) (2)
where [PT] is the concentration of target molecules associated with primer, T₀ is initial target concentration, c is the initial primer concentration, and t is primer annealing time. Assuming that each target molecule associated with primer is replicated to produce full size PCR product, the target amplification factor in a single PCR cycle is

$$Z = \frac{{T}_{0} + [ PT ]}{{T}_{0}} = 2 - {ⅇ}^{- {k}_{a} ct} \qquad ( 3 )$$

The total PCR amplification factor after n cycles is given by
F=Zⁿ=(2−e^{-k}^{ₐ}^{ct})ⁿ (4)

As it follows from equation 4, under the conditions where the primer annealing kinetics is the rate limiting step of PCR, the amplification factor should strongly depend on primer concentration. Thus, biased loci amplification, whether it is caused by individual association rate constants, primer extension steps or any other factors, can be corrected by adjusting primer concentration for each primer set in the multiplex PCR. The adjusted primer concentrations can be also used to correct biased performance of INVADER assay used for analysis of PCR pre-amplified loci. Employing this basic principle, the present invention has demonstrated a linear relationship between amplification efficiency and primer concentration and used this equation to balance primer concentrations of different amplicons, resulting in the equal amplification of ten different amplicons in Example 1. This technique may be employed on any size set of multiplex primer pairs.

II. Detection Assay Design

The following section describes detection assays that may be employed with the present invention. For example, many different assays may be used to determine the footprint on the target nucleic sequence, and then used as the detection assay run on the output of the multiplex PCR (or the detection assays may be run simultaneously with the multiplex PCR reaction).

There are a wide variety of detection technologies available for determining the sequence of a target nucleic acid at one or more locations. For example, there are numerous technologies available for detecting the presence or absence of SNPs. Many of these techniques require the use of an oligonucleotide to hybridize to the target. Depending on the assay used, the oligonucleotide is then cleaved, elongated, ligated, disassociated, or otherwise altered, wherein its behavior in the assay is monitored as a means for characterizing the sequence of the target nucleic acid. A number of these technologies are described in detail, in Section IV, below.

The present invention provides systems and methods for the design of oligonucleotides for use in detection assays. In particular, the present invention provides systems and methods for the design of oligonucleotides that successfully hybridize to appropriate regions of target nucleic acids (e.g., regions of target nucleic acids that do not contain secondary structure) under the desired reaction conditions (e.g., temperature, buffer conditions, etc.) for the detection assay. The systems and methods also allow for the design of multiple different oligonucleotides (e.g., oligonucleotides that hybridize to different portions of a target nucleic acid or that hybridize to two or more different target nucleic acids) that all function in the detection assay under the same or substantially the same reaction conditions. These systems and methods may also be used to design control samples that work under the experimental reaction conditions.

While the systems and methods of the present invention are not limited to any particular detection assay, the following description illustrates the invention when used in conjunction with the INVADER assay (Third Wave Technologies, Madison Wis.; See e.g., U.S. Pat. Nos. 5,846,717, 5,985,557, 5,994,069, and 6,001,567, PCT Publications WO 97/27214 and WO 98/42873, and de Arruda et al., Expert. Rev. Mol. Diagn. 2(5), 487-496 (2002), all of which are incorporated herein by reference in their entireties) to detect a SNP. The INVADER assay provides ease-of-use and sensitivity levels that, when used in conjunction with the systems and methods of the present invention, find use in detection panels, ASRs, and clinical diagnostics. One skilled in the art will appreciate that specific and general features of this illustrative example are generally applicable to other detection assays.

A. INVADER Assay

The INVADER assay provides means for forming a nucleic acid cleavage structure that is dependent upon the presence of a target nucleic acid and cleaving the nucleic acid cleavage structure so as to release distinctive cleavage products. 5′ nuclease activity, for example, is used to cleave the target-dependent cleavage structure and the resulting cleavage products are indicative of the presence of specific target nucleic acid sequences in the sample. When two strands of nucleic acid, or oligonucleotides, both hybridize to a target nucleic acid strand such that they form an overlapping invasive cleavage structure, as described below, invasive cleavage can occur. Through the interaction of a cleavage agent (e.g., a 5′ nuclease) and the upstream oligonucleotide, the cleavage agent can be made to cleave the downstream oligonucleotide at an internal site in such a way that a distinctive fragment is produced.

In some embodiments, the INVADER assay provides detections assays in which the target nucleic acid is reused or recycled during multiple rounds of hybridization with oligonucleotide probes and cleavage of the probes without the need to use temperature cycling (i.e., for periodic denaturation of target nucleic acid strands) or nucleic acid synthesis (i.e., for the polymerization-based displacement of target or probe nucleic acid strands). When a cleavage reaction is run under conditions in which the probes are continuously replaced on the target strand (e.g. through probe-probe displacement or through an equilibrium between probe/target association and disassociation, or through a combination comprising these mechanisms, (Reynaldo, et al., J. Mol. Biol. 97: 511-520 [2000]), multiple probes can hybridize to the same target, allowing multiple cleavages, and the generation of multiple cleavage products.

B. Oligonucleotide Design for the INVADER Assay

In some embodiments where an oligonucleotide is designed for use in the INVADER assay to detect a SNP, the sequence(s) of interest are entered into the INVADERCREATOR program (Third Wave Technologies, Madison, Wis.). As described above, sequences may be input for analysis from any number of sources, either directly into the computer hosting the INVADERCREATOR program, or via a remote computer linked through a communication network (e.g., a LAN, Intranet or Internet network). The program designs probes for both the sense and antisense strand. Strand selection is generally based upon the ease of synthesis, minimization of secondary structure formation, and manufacturability. In some embodiments, the user chooses the strand for sequences to be designed for. In other embodiments, the software automatically selects the strand. By incorporating thermodynamic parameters for optimum probe cycling and signal generation (Allawi and SantaLucia, Biochemistry, 36:10581 [1997]), oligonucleotide probes may be designed to operate at a pre-selected assay temperature (e.g., 63° C.). Based on these criteria, a final probe set (e.g., primary probes for 2 alleles and an INVADER oligonucleotide) is selected.

In some embodiments, the INVADERCREATOR system is a web-based program with secure site access that contains a link to BLAST (available at the National Center for Biotechnology Information, National Library of Medicine, National Institutes of Health website) and that can be linked to RNAstructure (Mathews et al., RNA 5:1458 [1999]), a software program that incorporates mfold (Zuker, Science, 244:48 [1989]). RNAstructure tests the proposed oligonucleotide designs generated by INVADERCREATOR for potential uni- and bimolecular complex formation. INVADERCREATOR is open database connectivity (ODBC)-compliant and uses the Oracle database for export/integration. The INVADERCREATOR system was configured with Oracle to work well with UNIX systems, as most genome centers are UNIX-based.

In some embodiments, the INVADERCREATOR analysis is provided on a separate server (e.g., a Sun server) so it can handle analysis of large batch jobs. For example, a customer can submit up to 2,000 SNP sequences in one email. The server passes the batch of sequences on to the INVADERCREATOR software, and, when initiated, the program designs detection assay oligonucleotide sets. In some embodiments, probe set designs are returned to the user within 24 hours of receipt of the sequences.

Each INVADER reaction includes at least two target sequence-specific, unlabeled oligonucleotides for the primary reaction: an upstream INVADER oligonucleotide and a downstream Probe oligonucleotide. The INVADER oligonucleotide is generally designed to bind stably at the reaction temperature, while the probe is designed to freely associate and disassociate with the target strand, with cleavage occurring only when an uncut probe hybridizes adjacent to an overlapping INVADER oligonucleotide. In some embodiments, the probe includes a 5′ flap or “arm” that is not complementary to the target, and this flap is released from the probe when cleavage occurs. In some embodiments, the released flap participates as an INVADER oligonucleotide in a secondary reaction.

The following discussion provides one example of how a user interface for an INVADERCREATOR program may be configured.

The user opens a work screen, e.g., by clicking on an icon on a desktop display of a computer (e.g., a Windows desktop). The user enters information related to the target sequence for which an assay is to be designed. In some embodiments, the user enters a target sequence. In other embodiments, the user enters a code or number that causes retrieval of a sequence from a database. In still other embodiments, additional information may be provided, such as the user's name, an identifying number associated with a target sequence, and/or an order number. In preferred embodiments, the user indicates (e.g. via a check box or drop down menu) that the target nucleic acid is DNA or RNA. In other preferred embodiments, the user indicates the species from which the nucleic acid is derived. In particularly preferred embodiments, the user indicates whether the design is for monoplex (i.e., one target sequence or allele per reaction) or multiplex (i.e., multiple target sequences or alleles per reaction) detection. When the requisite choices and entries are complete, the user starts the analysis process. In one embodiment, the user clicks a “Go Design It” button to continue.

In some embodiments, the software validates the field entries before proceeding. In some embodiments, the software verifies that any required fields are completed with the appropriate type of information. In other embodiments, the software verifies that the input sequence meets selected requirements (e.g., minimum or maximum length, DNA or RNA content). If entries in any field are not found to be valid, an error message or dialog box may appear. In preferred embodiments, the error message indicates which field is incomplete and/or incorrect. Once a sequence entry is verified, the software proceeds with the assay design.

In some embodiments, the information supplied in the order entry fields specifies what type of design will be created. In preferred embodiments, the target sequence and multiplex check box specify which type of design to create. Design options include but are not limited to SNP assay, Multiplexed SNP assay (e.g., wherein probe sets for different alleles are to be combined in a single reaction), Multiple SNP assay (e.g., wherein an input sequence has multiple sites of variation for which probe sets are to be designed), and Multiple Probe Arm assays.

In some embodiments, the INVADERCREATOR software is started via a Web Order Entry (WebOE) process (i.e., through an Intra/Internet browser interface) and these parameters are transferred from the WebOE via applet <param> tags, rather than entered through menus or check boxes.

In the case of Multiple SNP Designs, the user chooses two or more designs to work with. In some embodiments, this selection opens a new screen view (e.g., a Multiple SNP Design Selection view). In some embodiments, the software creates designs for each locus in the target sequence, scoring each, and presents them to the user in this screen view. The user can then choose any two designs to work with. In some embodiments, the user chooses a first and second design (e.g., via a menu or buttons) and clicks a “Go Design It” button to continue.

To select a probe sequence that will perform optimally at a pre-selected reaction temperature, the melting temperature (Tₘ) of the SNP to be detected is calculated using the nearest-neighbor model and published parameters for DNA duplex formation (Allawi and SantaLucia, Biochemistry, 36:10581 [1997]). In embodiments wherein the target strand is RNA, parameters appropriate for RNA/DNA heteroduplex formation may be used. Because the assay's salt concentrations are often different than the solution conditions in which the nearest-neighbor parameters were obtained (1M NaCl and no divalent metals), and because the presence and concentration of the enzyme influence optimal reaction temperature, an adjustment should be made to the calculated Tₘ to determine the optimal temperature at which to perform a reaction. One way of compensating for these factors is to vary the value provided for the salt concentration within the melting temperature calculations. This adjustment is termed a ‘salt correction’. As used herein, the term “salt correction” refers to a variation made in the value provided for a salt concentration for the purpose of reflecting the effect on a Tₘ calculation for a nucleic acid duplex of a non-salt parameter or condition affecting said duplex. Variation of the values provided for the strand concentrations will also affect the outcome of these calculations. By using a value of 0.5 M NaCl (SantaLucia, Proc Natl Acad Sci USA, 95:1460 [1998]) and strand concentrations of about 1 mM of the probe and 1 fM target, the algorithm for used for calculating probe-target melting temperature has been adapted for use in predicting optimal INVADER assay reaction temperature. For a set of 30 probes, the average deviation between optimal assay temperatures calculated by this method and those experimentally determined is about 1.5° C.

The length of the downstream probe to a given SNP is defined by the temperature selected for running the reaction (e.g., 63° C.). Starting from the position of the variant nucleotide on the target DNA (the target base that is paired to the probe nucleotide 5′ of the intended cleavage site), and adding on the 3′ end, an iterative procedure is used by which the length of the target-binding region of the probe is increased by one base pair at a time until a calculated optimal reaction temperature (Tₘ plus salt correction to compensate for enzyme effect) matching the desired reaction temperature is reached. The non-complementary arm of the probe is preferably selected to allow the secondary reaction to cycle at the same reaction temperature. The entire probe oligonucleotide is screened using programs such as mfold (Zuker, Science, 244: 48 [1989]) or Oligo 5.0 (Rychlik and Rhoads, Nucleic Acids Res, 17: 8543 [1989]) for the possible formation of dimer complexes or secondary structures that could interfere with the reaction. The same principles are also followed for INVADER oligonucleotide design. Briefly, starting from the position N on the target DNA, the 3′ end of the INVADER oligonucleotide is designed to have a nucleotide not complementary to either allele suspected of being contained in the sample to be tested. The mismatch does not adversely affect cleavage (Lyamichev et al., Nature Biotechnology, 17: 292 [1999]), and it can enhance probe cycling, presumably by minimizing coaxial stabilization effects between the two probes. Additional residues complementary to the target DNA starting from residue N−1 are then added in the 5′ direction until the stability of the INVADER oligonucleotide-target hybrid exceeds that of the probe (and therefore the planned assay reaction temperature), generally by 15-20° C.

It is one aspect of the assay design that the all of the probe sequences may be selected to allow the primary and secondary reactions to occur at the same optimal temperature, so that the reaction steps can run simultaneously. In an alternative embodiment, the probes may be designed to operate at different optimal temperatures, so that the reaction steps are not simultaneously at their temperature optima.

In some embodiments, the software provides the user an opportunity to change various aspects of the design including but not limited to: probe, target and INVADER oligonucleotide temperature optima and concentrations; blocking groups; probe arms; dyes, capping groups and other adducts; individual bases of the probes and targets (e.g., adding or deleting bases from the end of targets and/or probes, or changing internal bases in the INVADER and/or probe and/or target oligonucleotides). In some embodiments, changes are made by selection from a menu. In other embodiments, changes are entered into text or dialog boxes. In preferred embodiments, this option opens a new screen (e.g., a Designer Worksheet view).

In some embodiments, the software provides a scoring system to indicate the quality (e.g., the likelihood of performance) of the assay designs. In one embodiment, the scoring system includes a starting score of points (e.g., 100 points) wherein the starting score is indicative of an ideal design, and wherein design features known or suspected to have an adverse affect on assay performance are assigned penalty values. Penalty values may vary depending on assay parameters other than the sequences, including but not limited to the type of assay for which the design is intended (e.g., monoplex, multiplex) and the temperature at which the assay reaction will be performed. The following example provides an illustrative scoring criteria for use with some embodiments of the INVADER assay based on an intelligence defined by experimentation. Examples of design features that may incur score penalties include but are not limited to the following [penalty values are indicated in brackets, first number is for lower temperature assays (e.g., 62-64° C.), second is for higher temperature assays (e.g., 65-66° C.)]:

1. [100:100] 3′ end of INVADER oligonucleotide resembles the probe arm:

| | |
|---|---|
| ARM SEQUENCE: | PENALTY AWARDED IF INVADER ENDS IN: |

| | |
|---|---|
| Arm 1: CGCGCCGAGG | 5′...GAGGX or 5′...GAGGXX |
| Arm 2: ATGACGTGGCAGAC | 5′...CAGACX or 5′...CAGACXX |
| Arm 3: ACGGACGCGGAG | 5′...GGAGX or 5′...GGAGXX |
| Arm 4: TCCGCGCGTCC | 5′...GTCCX or 5′...GTCCXX |

2. [70:70] a probe has 5-base stretch (i.e., 5 of the same base in a row) containing the polymorphism;
3. [60:60] a probe has 5-base stretch adjacent to the polymorphism;
4. [50:50] a probe has 5-base stretch one base from the polymorphism;
5. [40:40] a probe has 5-base stretch two bases from the polymorphism;
6. [50:50] probe 5-base stretch is of Gs—additional penalty;
7. [100:100] a probe has 6-base stretch anywhere;
8. [90:90] a two or three base sequence repeats at least four times;
9. [100:100] a degenerate base occurs in a probe;
10. [60:90] probe hybridizing region is short (13 bases or less for designs 65-67° C.; 12 bases or less for designs 62-64° C.)
11. [40:90] probe hybridizing region is long (29 bases or more for designs 65-67° C., 28 bases or more for designs 62-64° C.)
12. [5:5] probe hybridizing region length—per base additional penalty
13. [80:80] Ins/Del design with poor discrimination in first 3 bases after probe arm
14. [100:100] calculated INVADER oligonucleotide Tm within 7.5° C. of probe target Tm (designs 65-67° C. with INVADER oligonucleotide less than ≦70.5° C., designs 62-64° C. with INVADER oligonucleotide≦69.5° C.
15. [20:20] calculated probes Tms differ by more than 2.0° C.
16. [100:100] a probe has calculated Tm 2° C. less than its target Tm
17. [10:10] target of one strand 8 bases longer than that of other strand
18. [30:30] INVADER oligonucleotide has 6-base stretch anywhere—initial penalty
19. [70:70] INVADER oligonucleotide 6-base stretch is of Gs—additional penalty
20. [15:15] probe hybridizing region is 14, 15 or 24-28 bases long (65-67° C.) or 13,14 or 26,27 bases long (62-64° C.)
21. [15:15] a probe has a 4-base stretch of Gs containing the polymorphism

In particularly preferred embodiments, temperatures for each of the oligonucleotides in the designs are recomputed and scores are recomputed as changes are made. In some embodiments, score descriptions can be seen by clicking a “descriptions” button. In some embodiments, a BLAST search option is provided. In preferred embodiments, a BLAST search is done by clicking a “BLAST Design” button. In some embodiments, this action brings up a dialog box describing the BLAST process. In preferred embodiments, the BLAST search results are displayed as a highlighted design on a Designer Worksheet.

In some embodiments, a user accepts a design by clicking an “Accept” button. In other embodiments, the program approves a design without user intervention. In preferred embodiments, the program sends the approved design to a next process step (e.g., into production; into a file or database). In some embodiments, the program provides a screen view (e.g., an Output Page), allowing review of the final designs created and allowing notes to be attached to the design. In preferred embodiments, the user can return to the Designer Worksheet (e.g., by clicking a “Go Back” button) or can save the design (e.g., by clicking a “Save It” button) and continue (e.g., to submit the designed oligonucleotides for production).

In some embodiments, the program provides an option to create a screen view of a design optimized for printing (e.g., a text-only view) or other export (e.g., an Output view). In preferred embodiments, the Output view provides a description of the design particularly suitable for printing, or for exporting into another application (e.g., by copying and pasting into another application). In particularly preferred embodiments, the Output view opens in a separate window.

The present invention is not limited to the use of the INVADERCREATOR software. Indeed, a variety of software programs are contemplated and are commercially available, including, but not limited to GCG Wisconsin Package (Genetics computer Group, Madison, Wis.) and Vector NTI (Informax, Rockville, Md.). Other detection assays may be used in the present invention.

1. Direct Sequencing Assays

In some embodiments of the present invention, variant sequences are detected using a direct sequencing technique. In these assays, DNA samples are first isolated from a subject using any suitable method. In some embodiments, the region of interest is cloned into a suitable vector and amplified by growth in a host cell (e.g., a bacteria). In other embodiments, DNA in the region of interest is amplified using PCR.

Following amplification, DNA in the region of interest (e.g., the region containing the SNP or mutation of interest) is sequenced using any suitable method, including but not limited to manual sequencing using radioactive marker nucleotides, or automated sequencing. The results of the sequencing are displayed using any suitable method. The sequence is examined and the presence or absence of a given SNP or mutation is determined.

2. PCR Assay

In some embodiments of the present invention, variant sequences are detected using a PCR-based assay. In some embodiments, the PCR assay comprises the use of oligonucleotide primers that hybridize only to the variant or wild type allele (e.g., to the region of polymorphism or mutation). Both sets of primers are used to amplify a sample of DNA. If only the mutant primers result in a PCR product, then the patient has the mutant allele. If only the wild-type primers result in a PCR product, then the patient has the wild type allele.

3. Fragment Length Polymorphism Assays

In some embodiments of the present invention, variant sequences are detected using a fragment length polymorphism assay. In a fragment length polymorphism assay, a unique DNA banding pattern based on cleaving the DNA at a series of positions is generated using an enzyme (e.g., a restriction enzyme or a CLEAVASE I [Third Wave Technologies, Madison, Wis.] enzyme). DNA fragments from a sample containing a SNP or a mutation will have a different banding pattern than wild type.

a. RFLP Assay

In some embodiments of the present invention, variant sequences are detected using a restriction fragment length polymorphism assay (RFLP). The region of interest is first isolated using PCR. The PCR products are then cleaved with restriction enzymes known to give a unique length fragment for a given polymorphism. The restriction-enzyme digested PCR products are generally separated by gel electrophoresis and may be visualized by ethidium bromide staining. The length of the fragments is compared to molecular weight markers and fragments generated from wild-type and mutant controls.

b. CFLP Assay

In other embodiments, variant sequences are detected using a CLEAVASE fragment length polymorphism assay (CFLP; Third Wave Technologies, Madison, Wis.; See e.g., U.S. Pat. Nos. 5,843,654; 5,843,669; 5,719,208; and 5,888,780; each of which is herein incorporated by reference). This assay is based on the observation that when single strands of DNA fold on themselves, they assume higher order structures that are highly individual to the precise sequence of the DNA molecule. These secondary structures involve partially duplexed regions of DNA such that single stranded regions are juxtaposed with double stranded DNA hairpins. The CLEAVASE I enzyme, is a structure-specific, thermostable nuclease that recognizes and cleaves the junctions between these single-stranded and double-stranded regions.

The region of interest is first isolated, for example, using PCR. In preferred emodiments, one or both strands are labeled. Then, DNA strands are separated by heating. Next, the reactions are cooled to allow intrastrand secondary structure to form. The PCR products are then treated with the CLEAVASE I enzyme to generate a series of fragments that are unique to a given SNP or mutation. The CLEAVASE enzyme treated PCR products are separated and detected (e.g., by denaturing gel electrophoresis) and visualized (e.g., by autoradiography, fluorescence imaging or staining). The length of the fragments is compared to molecular weight markers and fragments generated from wild-type and mutant controls.

4. Hybridization Assays

In preferred embodiments of the present invention, variant sequences are detected a hybridization assay. In a hybridization assay, the presence of absence of a given SNP or mutation is determined based on the ability of the DNA from the sample to hybridize to a complementary DNA molecule (e.g., a oligonucleotide probe). A variety of hybridization assays using a variety of technologies for hybridization and detection are available. A description of a selection of assays is provided below.

a. Direct Detection of Hybridization

In some embodiments, hybridization of a probe to the sequence of interest (e.g., a SNP or mutation) is detected directly by visualizing a bound probe (e.g., a Northern or Southern assay; See e.g., Ausabel et al. (eds.), Current Protocols in Molecular Biology, John Wiley & Sons, NY [1991]). In a these assays, genomic DNA (Southern) or RNA (Northern) is isolated from a subject. The DNA or RNA is then cleaved with a series of restriction enzymes that cleave infrequently in the genome and not near any of the markers being assayed. The DNA or RNA is then separated (e.g., on an agarose gel) and transferred to a membrane. A labeled (e.g., by incorporating a radionucleotide) probe or probes specific for the SNP or mutation being detected is allowed to contact the membrane under a condition or low, medium, or high stringency conditions. Unbound probe is removed and the presence of binding is detected by visualizing the labeled probe.

b. Detection of Hybridization Using “DNA Chip” Assays

In some embodiments of the present invention, variant sequences are detected using a DNA chip hybridization assay. In this assay, a series of oligonucleotide probes are affixed to a solid support. The oligonucleotide probes are designed to be unique to a given SNP or mutation. The DNA sample of interest is contacted with the DNA “chip” and hybridization is detected.

In some embodiments, the DNA chip assay is a GeneChip (Affymetrix, Santa Clara, Calif.; See e.g., U.S. Pat. Nos. 6,045,996; 5,925,525; and 5,858,659; each of which is herein incorporated by reference) assay. The GeneChip technology uses miniaturized, high-density arrays of oligonucleotide probes affixed to a “chip.” Probe arrays are manufactured by Affymetrix's light-directed chemical synthesis process, which combines solid-phase chemical synthesis with photolithographic fabrication techniques employed in the semiconductor industry. Using a series of photolithographic masks to define chip exposure sites, followed by specific chemical synthesis steps, the process constructs high-density arrays of oligonucleotides, with each probe in a predefined position in the array. Multiple probe arrays are synthesized simultaneously on a large glass wafer. The wafers are then diced, and individual probe arrays are packaged in injection-molded plastic cartridges, which protect them from the environment and serve as chambers for hybridization.

The nucleic acid to be analyzed is isolated, amplified by PCR, and labeled with a fluorescent reporter group. The labeled DNA is then incubated with the array using a fluidics station. The array is then inserted into the scanner, where patterns of hybridization are detected. The hybridization data are collected as light emitted from the fluorescent reporter groups already incorporated into the target, which is bound to the probe array. Probes that perfectly match the target generally produce stronger signals than those that have mismatches. Since the sequence and position of each probe on the array are known, by complementarity, the identity of the target nucleic acid applied to the probe array can be determined.

In other embodiments, a DNA microchip containing electronically captured probes (Nanogen, San Diego, Calif.) is utilized (See e.g., U.S. Pat. Nos. 6,017,696; 6,068,818; and 6,051,380; each of which are herein incorporated by reference). Through the use of microelectronics, Nanogen's technology enables the active movement and concentration of charged molecules to and from designated test sites on its semiconductor microchip. DNA capture probes unique to a given SNP or mutation are electronically placed at, or “addressed” to, specific sites on the microchip. Since DNA has a strong negative charge, it can be electronically moved to an area of positive charge.

First, a test site or a row of test sites on the microchip is electronically activated with a positive charge. Next, a solution containing the DNA probes is introduced onto the microchip. The negatively charged probes rapidly move to the positively charged sites, where they concentrate and are chemically bound to a site on the microchip. The microchip is then washed and another solution of distinct DNA probes is added until the array of specifically bound DNA probes is complete.

A test sample is then analyzed for the presence of target DNA molecules by determining which of the DNA capture probes hybridize, with complementary DNA in the test sample (e.g., a PCR amplified gene of interest). An electronic charge is also used to move and concentrate target molecules to one or more test sites on the microchip. The electronic concentration of sample DNA at each test site promotes rapid hybridization of sample DNA with complementary capture probes (hybridization may occur in minutes). To remove any unbound or nonspecifically bound DNA from each site, the polarity or charge of the site is reversed to negative, thereby forcing any unbound or nonspecifically bound DNA back into solution away from the capture probes. A laser-based fluorescence scanner is used to detect binding.

In still further embodiments, an array technology based upon the segregation of fluids on a flat surface (chip) by differences in surface tension (ProtoGene, Palo Alto, Calif.) is utilized (See e.g., U.S. Pat. Nos. 6,001,311; 5,985,551; and 5,474,796; each of which is herein incorporated by reference). Protogene's technology is based on the fact that fluids can be segregated on a flat surface by differences in surface tension that have been imparted by chemical coatings. Once so segregated, oligonucleotide probes are synthesized directly on the chip by ink-jet printing of reagents. The array with its reaction sites defined by surface tension is mounted on a X/Y translation stage under a set of four piezoelectric nozzles, one for each of the four standard DNA bases. The translation stage moves along each of the rows of the array and the appropriate reagent is delivered to each of the reaction site. For example, the A amidite is delivered only to the sites where amidite A is to be coupled during that synthesis step and so on. Common reagents and washes are delivered by flooding the entire surface and then removing them by spinning.

DNA probes unique for the SNP or mutation of interest are affixed to the chip using Protogene's technology. The chip is then contacted with the PCR-amplified genes of interest. Following hybridization, unbound DNA is removed and hybridization is detected using any suitable method (e.g., by fluorescence de-quenching of an incorporated fluorescent group).

In yet other embodiments, a “bead array” is used for the detection of polymorphisms (Illumina, San Diego, Calif.; See e.g., PCT Publications WO 99/67641 and WO 00/39587, each of which is herein incorporated by reference). Illumina uses a BEAD ARRAY technology that combines fiber optic bundles and beads that self-assemble into an array. Each fiber optic bundle contains thousands to millions of individual fibers depending on the diameter of the bundle. The beads are coated with an oligonucleotide specific for the detection of a given SNP or mutation. Batches of beads are combined to form a pool specific to the array. To perform an assay, the BEAD ARRAY is contacted with a prepared subject sample (e.g., DNA). Hybridization is detected using any suitable method.

c. Enzymatic Detection of Hybridization

In some embodiments of the present invention, hybridization is detected by enzymatic cleavage of specific structures (INVADER assay, Third Wave Technologies; See e.g., U.S. Pat. Nos. 5,846,717, 6,090,543; 6,001,567; 5,985,557; and 5,994,069; each of which is herein incorporated by reference). The INVADER assay detects specific DNA and RNA sequences by using structure-specific enzymes to cleave a complex formed by the hybridization of overlapping oligonucleotide probes. Elevated temperature and an excess of one of the probes enable multiple probes to be cleaved for each target sequence present without temperature cycling. These cleaved probes then direct cleavage of a second labeled probe. The secondary probe oligonucleotide can be 5′-end labeled with a fluorescent dye that is quenched by a second dye or other quenching moiety. Upon cleavage, the de-quenched dye-labeled product may be detected using a standard fluorescence plate reader, or an instrument configured to collect fluorescence data during the course of the reaction (i.e., a “real-time” fluorescence detector, such as an ABI 7700 Sequence Detection System, Applied Biosystems, Foster City, Calif.).

The INVADER assay detects specific mutations and SNPs in unamplified genomic DNA. In an embodiment of the INVADER assay used for detecting SNPs in genomic DNA, two oligonucleotides (a primary probe specific either for a SNP/mutation or wild type sequence, and an INVADER oligonucleotide) hybridize in tandem to the genomic DNA to form an overlapping structure. A structure-specific nuclease enzyme recognizes this overlapping structure and cleaves the primary probe. In a secondary reaction, cleaved primary probe combines with a fluorescence-labeled secondary probe to create another overlapping structure that is cleaved by the enzyme. The initial and secondary reactions can run concurrently in the same vessel. Cleavage of the secondary probe is detected by using a fluorescence detector, as described above. The signal of the test sample may be compared to known positive and negative controls.

In some embodiments, hybridization of a bound probe is detected using a TaqMan assay (PE Biosystems, Foster City, Calif.; See e.g., U.S. Pat. Nos. 5,962,233 and 5,538,848, each of which is herein incorporated by reference). The assay is performed during a PCR reaction. The TaqMan assay exploits the 5′-3′ exonuclease activity of DNA polymerases such as AMPLITAQ DNA polymerase. A probe, specific for a given allele or mutation, is included in the PCR reaction. The probe consists of an oligonucleotide with a 5′-reporter dye (e.g., a fluorescent dye) and a 3′-quencher dye. During PCR, if the probe is bound to its target, the 5′-3′ nucleolytic activity of the AMPLITAQ polymerase cleaves the probe between the reporter and the quencher dye. The separation of the reporter dye from the quencher dye results in an increase of fluorescence. The signal accumulates with each cycle of PCR and can be monitored with a fluorimeter.

In still further embodiments, polymorphisms are detected using the SNP-IT primer extension assay (Orchid Biosciences, Princeton, N.J.; See e.g., U.S. Pat. Nos. 5,952,174 and 5,919,626, each of which is herein incorporated by reference). In this assay, SNPs are identified by using a specially synthesized DNA primer and a DNA polymerase to selectively extend the DNA chain by one base at the suspected SNP location. DNA in the region of interest is amplified and denatured. Polymerase reactions are then performed using miniaturized systems called microfluidics. Detection is accomplished by adding a label to the nucleotide suspected of being at the SNP or mutation location. Incorporation of the label into the DNA can be detected by any suitable method (e.g., if the nucleotide contains a biotin label, detection is via a fluorescently labelled antibody specific for biotin).

III. Sequence Inputs and User Interfaces

Sequences may be input for analysis from any number of sources. In many embodiments, sequence information is entered into a computer. The computer need not be the same computer system that carries out in silico analysis. In some preferred embodiments, candidate target sequences may be entered into a computer linked to a communication network (e.g., a local area network, Internet or Intranet). In such embodiments, users anywhere in the world with access to a communication network may enter candidate sequences at their own locale. In some embodiments, a user interface is provided to the user over a communication network (e.g., a World Wide Web-based user interface), containing entry fields for the information required by the in silico analysis (e.g., the sequence of the candidate target sequence). The use of a Web based user interface has several advantages. For example, by providing an entry wizard, the user interface can ensure that the user inputs the requisite amount of information in the correct format. In some embodiments, the user interface requires that the sequence information for a target sequence be of a minimum length (e.g., 20 or more, 50 or more, 100 or more nucleotides) and be in a single format (e.g., FASTA). In other embodiments, the information can be input in any format and the systems and methods of the present invention edit or alter the input information into a suitable form for analysis. For example, if an input target sequence is too short, the systems and methods of the present invention search public databases for the short sequence, and if a unique sequence is identified, convert the short sequence into a suitably long sequence by adding nucleotides on one or both of the ends of the input target sequence. Likewise, if sequence information is entered in an undesirable format or contains extraneous, non-sequence characters, the sequence can be modified to a standard format (e.g., FASTA) prior to further in silico analysis. The user interface may also collect information about the user, including, but not limited to, the name and address of the user. In some embodiments, target sequence entries are associated with a user identification code.

In some embodiments, sequences are input directly from assay design software (e.g., the INVADERCREATOR software.

In preferred embodiments, each sequence is given an ID number. The ID number is linked to the target sequence being analyzed to avoid duplicate analyses. For example, if the in silico analysis determines that a target sequence corresponding to the input sequence has already been analyzed, the user is informed and given the option of by-passing in silico analysis and simply receiving previously obtained results.

Web-Ordering Systems and Methods

Users who wish to order detection assays, have detection assay designed, or gain access to databases or other information of the present invention may employ a electronic communication system (e.g., the Internet). In some embodiments, an ordering and information system of the present invention is connected to a public network to allow any user access to the information. In some embodiments, private electronic communication networks are provided. For example, where a customer or user is a repeat customer (e.g., a distributor or large diagnostic laboratory), the full-time dedicated private connection may be provided between a computer system of the customer and a computer system of the systems of the present invention. The system may be arranged to minimize human interaction. For example, in some embodiments, inventory control software is used to monitor the number and type of detection assays in possession of the customer. A query is sent at defined intervals to determine if the customer has the appropriate number and type of detection assay, and if shortages are detected, instructions are sent to design, produce, and/or deliver additional assays to the customer. In some embodiments, the system also monitors inventory levels of the seller and in preferred embodiments, is integrated with production systems to manage production capacity and timing.

In some embodiments, a user-friendly interface is provided to facilitate selection and ordering of detection assays. Because of the hundreds of thousands of detection assays available and/or polymorphisms that the user may wish to interrogate, the user-friendly interface allows navigation through the complex set of option. For example, in some embodiments, a series of stacked databases are used to guide users to the desired products. In some embodiments, the first layer provides a display of all of the chromosomes of an organism. The user selects the chromosome or chromosomes of interest. Selection of the chromosome provides a more detailed map of the chromosome, indicating banding regions on the chromosome. Selection of the desired band leads to a map showing gene locations. One or more additional layers of detail provide base positions of polymorphisms, gene names, genome database identification tags, annotations, regions of the chromosome with pre-existing developed detection assays that are available for purchase, regions where no pre-existing developed assays exist but that are available for design and production, etc. Selecting a region, polymorphism, or detection assay takes the user to an ordering interface, where information is collected to initiate detection assay design and/or ordering. In some embodiments, a search engine is provided, where a gene name, sequence range, polymorphism or other query is entered to more immediately direct the user to the appropriate layer of information.

In some embodiments, the ordering, design, and production systems are integrated with a finance system, where the pricing of the detection assay is determined by one or more factors: whether or not design is required, cost of goods based on the components in the detection assay, special discounts for certain customers, discounts for bulk orders, discounts for re-orders, price increases where the product is covered by intellectual property or contractual payment obligations to third parties, and price selection based on usage. For example, where detection assays are to be used for or are certified for clinical diagnostics rather than research applications, pricing is increased. In some embodiments, the pricing increase for clinical products occurs automatically. For example, in some embodiments, the systems of the present invention are linked to FDA, public publication, or other databases to determine if a product has been certified for clinical diagnostic or ASR use.

### EXAMPLES

The following examples are provided in order to demonstrate and further illustrate certain preferred embodiments and aspects of the present invention and are not to be construed as limiting the scope thereof.

In the experimental disclosure which follows, the following abbreviations apply: N (normal); M (molar); mM (millimolar); 1M (micromolar); mol (moles); mmol (millimoles); μmol (micromoles); nmol (nanomoles); pmol (picomoles); g (grams); mg (milligrams); μg (micrograms); ng (nanograms); l or L (liters); ml (milliliters); μl (microliters); cm (centimeters); mm (millimeters); μm (micrometers); nm (nanometers); DS (dextran sulfate); C (degrees Centigrade); and Sigma (Sigma Chemical Co., St. Louis, Mo.).

### Example 1

### Designing a 10-Plex (Manual): Test for Invader Assays

The following experimental example describes the manual design of amplification primers for a multiplex amplification reaction, and the subsequent detection of the amplicons by the INVADER assay.

Ten target sequences were selected from a set of pre-validated SNP-containing sequences, available in a TWT in-house oligonucleotide order entry database. Each target contains a single nucleotide polymorphism (SNP) to which an INVADER assay had been previously designed. The INVADER assay oligonucleotides were designed by the INVADER CREATOR software (Third Wave Technologies, Inc. Madison, Wis.), thus the footprint region in this example is defined as the INVADER “footprint”, or the bases covered by the INVADER and the probe oligonucleotides, optimally positioned for the detection of the base of interest, in this case, a single nucleotide polymorphism. About 200 nucleotides of each of the 10 target sequences were analyzed for the amplification primer design analysis, with the SNP base residing about in the center of the sequence.

Criteria of maximum and minimum probe length (defaults of 30 nucleotides and 12 nucleotides, respectively) were defined, as was a range for the probe melting temperature Tm of 50-60° C. In this example, to select a probe sequence that will perform optimally at a pre-selected reaction temperature, the melting temperature (Tₘ) of the oligonucleotide is calculated using the nearest-neighbor model and published parameters for DNA duplex formation (Allawi and SantaLucia, Biochemistry, 36:10581 [1997], herein incorporated by reference). Because the assay's salt concentrations are often different than the solution conditions in which the nearest-neighbor parameters were obtained (1M NaCl and no divalent metals), and because the presence and concentration of the enzyme influence optimal reaction temperature, an adjustment should be made to the calculated Tₘ to determine the optimal temperature at which to perform a reaction. One way of compensating for these factors is to vary the value provided for the salt concentration within the melting temperature calculations. This adjustment is termed a ‘salt correction’. The term “salt correction” refers to a variation made in the value provided for a salt concentration for the purpose of reflecting the effect on a Tₘ calculation for a nucleic acid duplex of a non-salt parameter or condition affecting said duplex. Variation of the values provided for the strand concentrations will also affect the outcome of these calculations. By using a value of 280 nM NaCl (SantaLucia, Proc Natl Acad Sci USA, 95:1460 [1998], herein incorporated by reference) and strand concentrations of about 10 pM of the probe and 1 fM target, the algorithm for used for calculating probe-target melting temperature has been adapted for use in predicting optimal primer design sequences.

Next, the sequence adjacent to the footprint region, both upstream and downstream were scanned and the first A or C was chosen for design start such that for primers described as 5′-N[x]-N[x−1]- . . . -N[4]-N[3]-N[2]-N[1]-3′, where N[1] should be an A or C. Primer complementarity was avoided by using the rule that: N[2]-N[1] of a given oligonucleotide primer should not be complementary to N[2]-N[1] of any other oligonucleotide, and N[3]-N[2]-N[1] should not be complementary to N[3]-N[2]-N[1] of any other oligonucleotide. If these criteria were not met at a given N[1], the next base in the 5′ direction for the forward primer or the next base in the 3′ direction for the reverse primer will be evaluated as an N[1] site. In the case of manual analysis, A/C rich regions were targeted in order to minimize the complementarity of 3′ ends.

In this example, an INVADER assay was performed following the multiplex amplification reaction. Therefore, a section of the secondary INVADER reaction oligonucleotide (the FRET oligonucleotide sequence) was also incorporated as criteria for primer design; the amplification primer sequence should be less than 80% homologous to the specified region of the FRET oligonucleotide.

All primers were synthesized according to standard oligonucleotide chemistry, desalted (by standard methods) and quantified by absorbance at A260 and diluted to 50 μM concentrated stock.

Multiplex PCR was then carried out using 10-plex PCR using equimolar amounts of primer (0.01 uM/primer) under the following conditions; 100 mM KCl, 3 mM MgCl₂, 10 mM Tris pH8.0, 200 uM dNTPs, 2.5 U Taq DNA polymerase, and 10 ng of human genomic DNA (hgDNA) template in a 50 ul reaction. The reaction was incubated for (94 C/30 sec, 50 C/44 sec.) for 30 cycles. After incubation, the multiplex PCR reaction was diluted 1:10 with water and subjected to INVADER analysis using INVADER Assay FRET Detection Plates, 96 well genomic biplex, 100 ng CLEAVASE VIII enzyme, INVADER assays were assembled as 15 ul reactions as follows; 1 ul of the 1:10 dilution of the PCR reaction, 3 ul of PPI mix, 5 ul of 22.5 mM MgC12, 6 ul of dH₂O, covered with 15 ul of CHILLOUT liquid wax. Samples were denatured in the INVADER biplex by incubation at 95 C for 5 min., followed by incubation at 63 C and fluorescence measured on a Cytofluor 4000 at various timepoints.

Using the following criteria to accurately make genotyping calls (FOZ_FAM+FOZ_RED−2>0.6), only 2 of the 10 INVADER assay calls can be made after 10 minutes of incubation at 63 C, and only 5 of the 10 calls could be made following an additional 50 min of incubation at 63 C (60 min.). At the 60 min time point, the variation between the detectable FOZ values is over 100 fold between the strongest signal (41646, FAM_FOZ+RED_FOZ−2=54.2, which is also is far outside of the dynamic range of the reader) and the weakest signal (67356, FAM_FOZ+RED_FOZ−2=0.2). Using the same INVADER assays directly against 100 ng of human genomic DNA (where equimolar amounts of each target would be available), all reads could be made with in the dynamic range of the reader and variation in the FOZ values was approximately seven fold between the strongest (53530, FAM_FOZ+RED_FOZ−2=3.1) and weakest (53530, FAM_FOZ+RED_FOZ−2=0.43) of the assays. This suggests that the dramatic discrepancies in FOZ values seen between different amplicons in the same multiplex PCR reaction is a function of biased amplification, and not variability attributable to INVADER assay. Under these conditions, FOZ values generated by different INVADER assays are directly comparable to one another and can reliably be used as indicators of the efficiency of amplification.

Estimation of amplification factor of a given amplicon using FOZ values. In order to estimate the amplification factor (F) of a given amplicon, the FOZ values of the INVADER assay can be used to estimate amplicon abundance. The FOZ of a given amplicon with unknown concentration at a given time (FOZm) can be directly compared to the FOZ of a known amount of target (e.g. 100 ng of genomic DNA=30,000 copies of a single gene) at a defined point in time (FOZ₂₄₀, 240 min) and used to calculate the number of copies of the unknown amplicon. In equation 1, FOZm represents the sum of RED_FOZ and FAM_FOZ of an unknown concentration of target incubated in an INVADER assay for a given amount of time (m). FOZ₂₄₀ represents an empirically determined value of RED_FOZ (using INVADER assay 41646), using for a known number of copies of target (e.g. 100 ng of hgDNA≅30,000 copies) at 240 minutes.
F=((FOZₘ−1)*500/(FOZ₂₄₀−1))*(240/m)\hat{ }2 (equation 1a)

Although equation 1a is used to determine the linear relationship between primer concentration and amplification factor F, equation 1a′ is used in the calculation of the amplification factor F for the 10-plex PCR (both with equimolar amounts of primer and optimized concentrations of primer), with the value of D representing the dilution factor of the PCR reaction. In the case of a 1:3 dilution of the 50 ul multiplex PCR reaction. D=0.3333.
F=((FOZₘ−2)*500/(FOZ₂₄₀−1)*D)*(240/m)\hat{ }2 (equation 1a′)

Although equations 1a and 1a′ will be used in the description of the 10-plex multiplex PCR, a more correct adaptation of this equation was used in the optimization of primer concentrations in the 107-plex PCR. In this case, FOZ₂₄=the average of FAM_FOZ₂₄₀+RED_FOZ₂₄₀ over the entire INVADER MAP plate using hgDNA as target (FOZ₂₄₀±3.42) and the dilution factor D is set to 0.125.
F=((FOZₘ−2)*500/(FOZ₂₄₀−2)*D)*(240/m)\hat{ }2 (equation 1b)

It should be noted that in order for the estimation of amplification factor F to be more accurate, FOZ values should be within the dynamic range of the instrument on which the reading are taken. In the case of the Cytofluor 4000 used in this study, the dynamic range was between about 1.5 and about 12 FOZ.

Section 3. Linear Relationship Between Amplification Factor and Primer Concentration.

In order to determine the relationship between primer concentration and amplification factor (F), four distinct uniplex PCR reactions were run at using primers 1117-70-17 and 1117-70-18 at concentrations of 0.01 uM, 0.012 uM, 0.014 uM, 0.020 uM respectively. The four independent PCR reactions were carried out under the following conditions; 100 mM KCl, 3 mM MgCl, 10 mM Tris pH 8.0, 200 uM dNTPs using 10 ng of hgDNA as template. Incubation was carried out at (94 C/30 sec., 50 C/20 sec.) for 30 cycles. Following PCR, reactions were diluted 1:10 with water and run under standard conditions using INVADER Assay FRET Detection Plates, 96 well genomic biplex, 100 ng CLEAVASE VIII enzyme. Each 15 ul reaction was set up as follows; 1 ul of 1:10 diluted PCR reaction, 3 ul of the PPI mix SNP#47932, 5 ul 22.5 mM MgCl2, 6 ul of water, 15 ul of CHILLOUT liquid wax. The entire plate was incubated at 95 C for 5 min, and then at 63 C for 60 min at which point a single read was taken on a Cytofluor 4000 fluorescent plate reader. For each of the four different primer concentrations (0.01 uM, 0.012 uM, 0.014 uM, 0.020 uM) the amplification factor F was calculated using equation 1a, with FOZm=the sum of FOZ_FAM and FOZ_RED at 60 minutes, m=60, and FOZ₂₄₀=1.7. In plotting the primer concentration of each reaction against the log of the amplification factor Log(F), a strong linear relationship was noted. Using these data points, the formula describing the linear relationship between amplification factor and primer concentration is described in equation 2:
Y=1.684X+2.6837 (equation 2a)

Using equation 2, the amplification factor of a given amplicon Log(F)=Y could be manipulated in a predictable fashion using a known concentration of primer (X). In a converse manner, amplification bias observed under conditions of equimolar primer concentrations in multiplex PCR, could be measured as the “apparent” primer concentration (X) based on the amplification factor F. In multiplex PCR, values of “apparent” primer concentration among different amplicons can be used to estimate the amount of primer of each amplicon required to equalize amplification of different loci:
X=(Y−2.6837)/1.68 (equation 2b)
Section 4. Calculation of Apparent Primer Concentrations from a Balanced Multiplex Mix.

As described in a previous section, primer concentration can directly influence the amplification factor of given amplicon. Under conditions of equimolar amounts of primers, FOZm readings can be used to calculate the “apparent” primer concentration of each amplicon using equation 2. Replacing Y in equation 2 with log(F) of a given amplification factor and solving for X, gives an “apparent” primer concentration based on the relative abundance of a given amplicon in a multiplex reaction. Using equation 2 to calculate the “apparent” primer concentration of all primers (provided in equimolar concentration) in a multiplex reaction provides a means of normalizing primer sets against each other. In order to derive the relative amounts of each primer that should be added to an “Optimized” multiplex primer mix R, each of the “apparent” primer concentrations should be divided into the maximum apparent primer concentration (Xₘₐₓ), such that the strongest amplicon is set to a value of 1 and the remaining amplicons to values equal or greater than 1
R[n]=Xmax/X[n] (equation 3)

Using the values of R[n] as an arbitrary value of relative primer concentration, the values of R[n] are multiplied by a constant primer concentration to provide working concentrations for each primer in a given multiplex reaction. In the example shown, the amplicon corresponding to SNP assay 41646 has an R[n] value equal to 1. All of the R[n] values were multiplied by 0.01 uM (the original starting primer concentration in the equimolar multiplex PCR reaction) such that lowest primer concentration is R[n] of 41646 which is set to 1, or 0.01 uM. The remaining primer sets were also proportionally increased. The results of multiplex PCR with the “optimized” primer mix are described below.

Section 5 Using Optimized Primer Concentrations in Multiplex PCR, Variation in FOZ's Among 10 INVADER Assays are Greatly Reduced.

Multiplex PCR was carried out using 10-plex PCR using varying amounts of primer based on the volumes (X[max] was SNP41646, setting 1×=0.01 uM/primer). Multiplex PCR was carried out under conditions identical to those used in with equimolar primer mix; 100 mM KCl, 3 mM MgCl, 10 mM Tris pH8.0, 200 uM dNTPs, 2.5 U taq, and 10 ng of hgDNA template in a 50 ul reaction. The reaction was incubated for (94 C/30 sec, 50 C/44 sec.) for 30 cycles. After incubation, the multiplex PCR reaction was diluted 1:10 with water and subjected to INVADER analysis. Using INVADER Assay FRET Detection Plates, (96 well genomic biplex, 100 ng CLEAVSE VIII enzyme), reactions were assembled as 15 ul reactions as follows; 1 ul of the 1:10 dilution of the PCR reaction, 3 ul of the appropriate PPI mix, 5 ul of 22.5 mM MgCl2, 6 ul of dH₂O. An additional 15 ul of CHILL OUT was added to each well, followed by incubation at 95 C for 5 min. Plates were incubated at 63 C and fluorescence measured on a Cytofluor 4000 at 10 min.

Using the following criteria to accurately make genotyping calls (FOZ_FAM+FOZ_RED−2>0.6), all 10 of 10 (100%) INVADER calls can be made after 10 minutes of incubation at 63 C. In addition, the values of FAM+RED−2 (an indicator of overall signal generation, directly related to amplification factor (see equation 2)) varied by less than seven fold between the the lowest signal (67325, FAM+RED−2=0.7) and the highest (47892, FAM+RED−2=4.3).

### Example 2

### Design of 101-Plex PCR Using the Software Application

Using the TWT Oligo Order Entry Database, 144 sequences of less than 200 nucleotides in length were obtained, with SNPs annotated using brackets to indicate the SNP position for each sequence (e.g. NNNNNNN[N_{(wt)}/N₍ₘₜ₎]NNNNNNNN). In order to expand sequence data flanking the SNP of interest, sequences were expanded to approximately 1 kB in length (500 nts flanking each side of the SNP) using BLAST analysis. Of the 144 starting sequences, 16 could not expanded by BLAST, resulting in a final set of 128 sequences expanded to approximately 1 kB length. These expanded sequences were provided to the user in Excel format with the following information for each sequence; (1) TWT Number, (2) Short Name Identifier, and (3) sequence. The Excel file was converted to a comma delimited format and used as the input file for Primer Designer INVADER CREATOR v1.3.3. software (this version of the program does not screen for FRET reactivity of the primers, nor does it allow the user to specify the maximum length of the primer). INVADER CREATOR Primer Designer v1.3.3., was run using default conditions (e.g. minimum primer size of 12, maximum of 30), with the exception of Tm_{low} which was set to 60 C. The output file contained 128 primer sets (256 primers), four of which were thrown out due to excessively long primer sequences (SNP # 47854, 47889, 54874, 67396), leaving 124 primers sets (248 primers) available for synthesis. The remaining primers were synthesized using standard procedures at the 200 nmol scale and purified by desalting. After synthesis failures, 107 primer sets were available for assembly of an equimolar 107-plex primer mix (214 primers). Of the 107 primer sets available for amplification, only 101 were present on the INVADER MAP plate to evaluate amplification factor.

Multiplex PCR was carried out using 101-plex PCR using equimolar amounts of primer (0.025 uM/primer) under the following conditions; 100 mM KCl, 3 mM MgCl, 10 mM Tris pH8.0, 200 uM dNTPs, and 10 ng of human genomic DNA (hgDNA) template in a 50 ul reaction. After denaturation at 95 C for 10 min, 2.5 units of Taq was added and the reaction incubated for (94 C/30 sec, 50 C/44 sec.) for 50 cycles. After incubation, the multiplex PCR reaction was diluted 1:24 with water and subjected to INVADER assay analysis using INVADER MAP detection platform. Each INVADER MAP assay was run as a 6 ul reaction as follows; 3 ul of the 1:24 dilution of the PCR reaction (total dilution 1:8 equaling D=0.125), 3 ul of 15 mM MgCl2 covered with covered with 6 ul of CHILLOUT. Samples were denatured in the INVADER MAP plate by incubation at 95 C for 5 min., followed by incubation at 63 C and fluorescence measured on a Cytofluor 4000 (384 well reader) at various timepoints over 160 minutes. Analysis of the FOZ values calculated at 10, 20, 40, 80, 160 min. shows that correct calls (compared to genomic calls of the same DNA sample) could be made for 94 of the 101 amplicons detectable by the INVADER MAP platform. This provides proof that the INVADER CREATOR Primer Designer software can create primer sets which function in highly multiplex PCR.

In using the FOZ values obtained throughout the 160 min. time course, amplification factor F and R[n] were calculated for each of the 101 amplicons. R[nmax] was set at 1.6, which although Low end corrections were made for amplicons which failed to provide sufficient FOZm signal at 160 min., assigning an arbitrary value of 12 for R[n]. High end corrections for amplicons whose FOZm values at the 10 min. read, an R[n] value of 1 was arbitrarily assigned. Optimized primer concentrations of the 101-plex were calculated using the basic principles outlined in the 10-plex example and equation 1b, with an R[n] of 1 corresponding to 0.025 uM primer (see FIG. 15 for various primer concentrations). Multiplex PCR was under the following conditions; 100 mM KCl, 3 mM MgCl, 10 mM Tris pH8.0, 200 uM dNTPs, and 10 ng of human genomic DNA (hgDNA) template in a 50 ul reaction. After denaturation at 95 C for 10 min, 2.5 units of Taq was added and the reaction incubated for (94 C/30 sec, 50 C/44 sec.) for 50 cycles. After incubation, the multiplex PCR reaction was diluted 1:24 with water and subjected to INVADER analysis using INVADER MAP detection platform. Each INVADER MAP assay was run as a 6 ul reaction as follows; 3 ul of the 1:24 dilution of the PCR reaction (total dilution 1:8 equaling D=0.125), 3 ul of 15 mM MgCl2 covered with covered with 6 ul of CHILLOUT. Samples were denatured in the INVADER MAP plate by incubation at 95 C for 5 min., followed by incubation at 63 C and fluorescence measured on a Cytofluor 4000 (384 well reader) at various timepoints over 160 minutes. Analysis of the FOZ values was carried out at 10, 20, and 40 min. and compared to calls made directly against the genomic DNA. Comparison was made between calls made at 10 min. with a 101-plex PCR with the equimolar primer concentrations versus calls that were made at 10 min. with a 101-plex PCR run under optimized primer concentrations. Under equimolar primer concentration, multiplex PCR results in only 50 correct calls at the 10 min time point, where under optimized primer concentrations multiplex PCR results in 71 correct calls, resulting in a gain of 21 (42%) new calls. Although all 101 calls could not be made at the 10 min timepoint, 94 calls could be made at the 40 min. timepoint suggesting the amplification efficiency of the majority of amplicons had improved. Unlike the 10-plex optimization that only required a single round of optimization, multiple rounds of optimization may be required for more complex multiplexing reactions to balance the amplification of all loci.

### Example 3

### Use of the Invader Assay to Determine Amplification Factor of PCR

The INVADER assay can be used to monitor the progress of amplification during PCR reactions, i.e., to determine the amplification factor F that reflects efficiency of amplification of a particular amplicon in a reaction. In particular, the INVADER assay can be used to determine the number of molecules present at any point of a PCR reaction by reference to a standard curve generated from quantified reference DNA molecules. The amplification factor F is measured as a ratio of PCR product concentration after amplification to initial target concentration. This example demonstrates the effect of varying primer concentration on the measured amplification factor.

PCR reactions were conducted for variable numbers of cycles in increments of 5, i.e., 5, 10, 15, 20, 25, 30, so that the progress of the reaction could be assessed using the INVADER assay to measure accumulated product. The reactions were diluted serially to assure that the target amounts did not saturate the INVADER assay, i.e., so that the measurements could be made in the linear range of the assay. INVADER assay standard curves were generated using a dilution series containing known amounts of the amplicon. This standard curve was used to extrapolate the number of amplified DNA fragments in PCR reactions after the indicated number of cycles. The ratio of the number of molecules after a given number of PCR cycles to the number present prior to amplification is used to derive the amplification factor, F, of each PCR reaction.

PCR Reactions

PCR reactions were set up using equimolar amounts of primers (e.g., 0.02 μM or 0.1 μM primers, final concentration). Reactions at each primer concentration were set up in triplicate for each level of amplification tested, i.e., 5, 10, 15, 20, 25, and 30 PCR cycles. One master mix sufficient for 6 standard PCR reactions (each in triplicate×2 primer concentrations) plus 2 controls×6 tests (5, 10, 15, 20, 25, or 30 cycles of PCR) plus enough for extra reactions to allow for overage.

Serial dilutions of PCR reaction products In order to ensure that the amount of PCR product added as target to the INVADER assay reactions would not exceed the dynamic range of the real time assay on the PERSEPTIVE BIOSYSTEMS CYTOFLUOR 4000, the PCR reaction products were diluted prior to addition to the INVADER assays. An initial 20-fold dilution was made of each reaction, followed by subsequent five-fold serial dilutions.

To create standards, amplification products generated with the same primers used in the tests of different numbers of cycles were isolated from non-denaturing polyacrylimide gels using standard methods and quantified using the PICOGREEN assay. A working stock of 200 pM was created, and serial dilutions of these concentration standards were created in dH₂O containing tRNA at 30 ng/μl to yield a series with final amplicon concentrations of 0.5, 1, 2.5, 6.25, 15.62, 39, and 100 fM.

INVADER Assay Reactions

Appropriate dilutions of each PCR reaction and the no target control were made in triplicate, and tested in standard, singlicate INVADER assay reactions. One master mix was made for all INVADER assay reactions. In all, there were 6 PCR cycle conditions×24 individual test assays [(1 test of triplicate dilutions×2 primer conditions×3 PCR replicates)=18+6 no target controls]. In addition, there were 7 dilutions of the quantified amplicon standards and 1 no target control in the standard series. The standard series was analyzed in replicate on each of two plates, for an additional 32 INVADER assays. The total number of INVADER assays is 6×24+32=176. The master mix included coverage for 32 reactions. INVADER assay master mix and comprised the following standard components:FRET buffer/Cleavase XI/Mg/PPI mix for 192 plus 16 wells.

The following oligonucleotides were included in the PPI mix.
0.25 μM INVADER for assay 2 (GAAGCGGCGCCGGTTACCACCA)
2.5 μM A Probe for assay 2 (CGCGCCGAGGTGGTTGAGCAATTCCAA)
2.5 μM G Probe for assay 2 (ATGACGTGGCAGACCGGTTGAGCAATTCCA)

All wells were overlaid with 15 μl mineral oil, incubated at 95° C. 5 min, then at 63° C. read at various intervals, eg. 20, 40, 80, or 160 min, depending on the level of signal generated. The reaction plate was read on a CytoFluor® Series 4000 Fluorescence Multi-Well Plate Reader. The settings used were: 485/20 nm excitation/bandwidth and 530/25 nm emission/bandwidth for F dye detection, and 560/20 nm excitation/bandwidth and 620/40 nm emission/bandwidth for R dye detection. The instrument gain was set for each dye so that the No Target Blank produced between 100-200 Absolute Fluorescence Units (AFUs).

Results:

When the results of the triplicate INVADER assays were diagrammed in a plot of log₁₀ of amplification factor (y-axis) as a function of cycle number (x-axis), the PCR product concentration was estimated from the INVADER assays by extrapolation to the standard curve. The data from the replicate assays were not averaged but instead were presented as multiple, overlapping points in the figure.

These results indicate that the PCR reactions were exponential over the range of cycles tested. The use of different primer concentrations resulted in different slopes such that the slope generated from INVADER assay analysis of PCR reactions carried out with the higher primer concentration (0.1 μM) is steeper than that with the lower (0.02 μM) concentration. In addition, the slope obtained using 0.1 μM approaches that anticipated for perfect doubling (0.301). The amplification factors from the PCR reactions at each primer concentration were obtained from the slopes:
For 0.1 μM primers, slope=0.286; amplification factor: 1.93
For 0.02 μM primers, slope=0.218; amplification factor: 1.65.
   The lines do not appear to extend to the origin but rather intercept the X-axis between 0 and 5 cycles, perhaps reflective of errors in estimating the starting concentration of human genomic DNA.

Thus, these data show that primer concentration affects the extent of amplification during the PCR reaction. These data further demonstrate that the INVADER assay is an effective tool for monitoring amplification throughout the PCR reaction.

### Example 4

### Dependence of Amplification Factor on Primer Concentration

This example demonstrates the correlation between amplification factor, F, and primer concentration, c. In this experiment, F was determined for 2 alleles from each of 6 SNPs amplified in monoplex PCR reactions, each at 4 different primer concentrations, hence 6 primer pairs×2 genomic samples×4 primer concentrations=48 PCR reactions.

Whereas the effect of PCR cycle number was tested on a single amplified region, at two primer concentrations, in Example 3, in this example, all test PCR reactions were run for 20 cycles, but the effect of varying primer concentration was studied at 4 different concentration levels: 0.01 μM, 0.025 μM, 0.05 μM, 0.1 μM. Furthermore, this experiment examines differences in amplification of different genomic regions to investigate (a) whether different genomic regions are amplified to different extents (i.e. PCR bias) and (b) how amplification of different genomic regions depends on primer concentration.

As in Example 3, F was measured by generating a standard curve for each locus using a dilution series of purified, quantified reference amplicon preparations. In this case, 12 different reference amplicons were generated: one for each allele of the SNPs contained in the 6 genomic regions amplified by the primer pairs. Each reference amplicon concentration was tested in an INVADER assay, and a standard curve of fluorescence counts versus amplicon concentration was created. PCR reactions were also run on genomic DNA samples, the products diluted, and then tested in an INVADER assay to determine the extent of amplification, in terms of number of molecules, by comparison to the standard curve.

a. Generation of Standard Curves Using Quantified Reference Amplicons

A total of 8 genomic DNA samples isolated from whole blood were screened in standard biplex INVADER assays to determine their genotypes at 24 SNPs in order to identify samples homozygous for the wild-type or variant allele at a total of 6 different loci.

Once these loci were identified, wild-type and variant genomic DNA samples were analyzed in separate PCR reactions with primers flanking the genomic region containing each SNP. At each SNP, one allele reported to FAM dye and one to RED.

Suitable genomic DNA preparations were then amplified in standard individual, monoplex PCR reactions to generate amplified fragments for use as PCR reference standards as described in Example 3.

Following PCR, amplified DNA was gel isolated using standard methods and previously quantified using the PICOGREEN assay. Serial dilutions of these concentration standards were created as follows:

Each purified amplicon was diluted to create a working stock at a concentration of 200 pM. These stocks were then serially diluted as follows. A working stock solution of each amplicon was prepared with a concentration of 1.25 pM in dH₂O containing tRNA at 30 ng/μl. The working stock was diluted in 96-well microtiter plates and then serially diluted to yield the following final concentrations in the INVADER assay: 1, 2.5, 6.25, 15.6, 39, 100, and 250 fM. One plate was prepared for the amplicons to be detected in the INVADER assay using probe oligonucleotides reporting to FAM dye and one plate for those to be tested with probe oligonucleotides reporting to RED dye. All amplicon dilutions were analyzed in duplicate.

Aliquots of 100 μl were transferred, in this layout, to 96 well MJ Research plates and denatured for 5 min at 95° C. prior to addition to INVADER assays.

b. PCR Amplification of Genomic Samples at Different Primer Concentrations.

PCR reactions were set up for individual amplification of the 6 genomic regions described in the previous example on each of 2 alleles at 4 different primer concentrations, for a total of 48 PCR reactions. All PCRs were run for 20 cycles. The following primer concentrations were tested: 0.01 μM, 0.025 μM, 0.05 μM, and 0.1 μM. A master mix for all 48 reactions was prepared according to standard procedures, with the exception of the modified primer concentrations, plus overage for an additional 23 reactions (16 reactions were prepared but not used, and overage of 7 additional reactions was prepared).

c. Dilution of PCR Reactions

Prior to analysis by the INVADER assay, it was necessary to dilute the products of the PCR reactions, as described in Examples 1 and 2. Serial dilutions of each of the 48 PCR reactions were made using one 96-well plate for each SNP. The left half of the plate contained the SNPs to be tested with probe oligonucleotides reporting to FAM; the right half, with probe oligonucleotides reporting to RED. The initial dilution was 1:20; asubsequent dilutions were 1:5 up to 1:62,500.

d. INVADER Assay Analysis of PCR Dilutions and Reference Amplicons

INVADER analysis was carried out on all dilutions of the products of each PCR reaction as well as the indicated dilutions of each quantified reference amplicon (to generate a standard curve for each amplicon) in standard biplex INVADER assays.

All wells were overlaid with 15 μl of mineral oil. Samples were heated to 95° C. for 5 min to denature and then incubated at 64° C. Fluorescence measurements were taken at 40 and 80 minutes in a CytoFluor® 4000 fluorescence plate reader (Applied Biosystems, Foster City, Calif.). The settings used were: 485/20 nm excitation/bandwidth and 530/25 nm emission/bandwidth for F dye detection, and 560/20 nm excitation/bandwidth and 620/40 nm emission/bandwidth for R dye detection. The instrument gain was set for each dye so that the No Target Blank produced between 100-200 Absolute Fluorescence Units (AFUs). The raw data is that generated by the device/instrument used to measure the assay performance (real-time or endpoint mode).

These results indicate that the dependence of lnF on c demonstrates different amplification rates for the 12 PCRs under the same reaction conditions, although the difference is much smaller within each pair of targets representing the same SNP. The amplification factor strongly depends on c at low primer concentrations with a trend to plateau at higher primer concentrations. This phenomenon can be explained in terms of the kinetics of primer annealing. At high primer concentrations, fast annealing kinetics ensures that primers are bound to all targets and maximum amplification rate is achieved, on the contrary, at low primer concentrations the primer annealing kinetics become a rate limiting step decreasing F.

This analysis suggests that plotting amplification factor as a function of primer concentration in ln

$$( 2 - {F}^{\frac{1}{n}} )$$

vs. c coordinates should produce a straight line with a slope −kₐtₐ. Re-plotting of the data in the in

$$( 2 - {F}^{\frac{1}{n}} )$$

vs. c coordinates demonstrates the expected linear dependence for low primer concentrations (low amplification factor) which deviates from the linearity at 0.1 μM primer concentration (F is 10⁵ or larger) due to lower than expected amplification factor. The kₐtₐ. values can be calculated for each PCR using the following equation.
F=zⁿ=(2−e^{-k}^{ₐ}^{ct}^{ₐ})ⁿ

### Example 5

### Invader Assay Analysis of 192-Plex PCR Reaction

This example describes the use of the INVADER assay to detect the products of a highly multiplexed PCR reaction designed to amplify 192 distinct loci in the human genome.

Genomic DNA Extraction

Genomic DNA was isolated from 5 mls of whole blood and purified using the Autopure, manufactured by Gentra Systems, Inc. (Minneapolis, Minn.). The purified DNA was in 500 μl of dH₂O.

Primer Design

Forward and reverse primer sets for the 192 loci were designed using Primer Designer, version 1.3.4 (See Primer Design section above, including FIG. 8). Target sequences used for INVADER designs, with no more than 500 bases flanking the relevant SNP site, were converted into a comma-delimited text file for use as an input file for PrimerDesigner. PrimerDesigner was run using default parameters, with the exception of oligo Tₘ, which was set at 60° C.

Primer Synthesis

Oligonucleotide primers were synthesized using standard procedures in a Polyplex (GeneMachines, San Carlos, Calif.). The scale was 0.2 μmole, desalted only (not purified) on NAP-10 and not dried down.

PCR Reactions

Two master mixes were created. Master mix 1 contained primers to amplify loci 1-96; master mix 2, 97-192. The mixes were made according to standard procedures and contained standard components. All primers were present at a final concentration of 0.025 μM, with KCl at 100 mM, and MgCl at 3 mM. PCR cycling conditions were as follows in a MJ PTC-100 thermocycler (MJ Research, Waltham, Mass.): 95° C. for 15 min; 94° C. for 30 sec, then 55° C. 44 sec×50 cycles.

Following cycling, all 4 PCR reactions were combined and aliquots of 3 μL were distributed into a 384 deep-well plate using a CYBI-well 2000 automated pipetting station (CyBio AG, Jena, Germany). This instrument makes individual reagent additions to each well of a 384-well microplate. The reagents to be added are themselves arrayed in 384-well deep half plates.

INVADER Assay Reactions

INVADER assays were set up using the CYBI-well 2000. Aliquots of 3 μl of the genomic DNA target were added to the appropriate wells. No target controls were comprised of 3 μl of Te (10 mM Tris, pH 8.0, 0.1 mM EDTA). The reagents for use in the INVADER assays were standard PPI mixes, buffer, FRET oligonucleotides, and Cleavase VIII enzyme and were added individually to each well by the CYBI-well 2000.

Following the reagent additions, 6 μl of mineral oil were overlaid in each well. The plates were heated in a MJ PTC-200 DNA ENGINE thermocycler (MJ Research) to 95° C. for 5 minutes then cooled to the incubation temperature of 63° C. Fluorescence was read after 20 minutes and 40 minutes using the Safire microplate reader (Tecan, Zurich, Switzerland) using the following settings. 495/5 nm excitation/bandwidth and 520/5 nm emission/bandwidth for F dye detection; and 600/5 nm emission/bandwidth, 575/5 nm excitation/bandwidth Z position, 5600 μs; number of flashes, 10; lag time, 0; integration time, 40 μsec for R dye detection. Gain was set for F dye at 90 nm and R dye at 120. The raw data is that generated by the device/instrument used to measure the assay performance (real-time or endpoint mode).

Of the 192 reactions, genotype calls could be made for 157 after 20 minutes and 158 after 40 minutes, or a total of 82%. For 88 of the assays, genotyping results were available for comparison from data obtained previously using either monoplex PCR followed by INVADER analysis or INVADER results obtained directly from analysis of genomic DNA. For 69 results, no corroborating genotype results were available.

This example shows that it is possible to amplify more than 150 loci in a single multiplexed PCR reaction. This example further shows that the amount of each amplified fragment generated in such a multiplexed PCR reaction is sufficient to produce discernable genotype calls when used as a target in an INVADER assay. In addition, many of the amplicons generated in this multiplex PCR assay gave high signal, measured as FOZ, in the INVADER assay, while some gave such low signal that no genotype call could be made. Still others amplicons were present at such low levels, or not at all, that they failed to yield any signal in the INVADER assay.

### Example 6

### Optimization of Primer Concentration to Improve Performance of Highly Multiplexed PCR Reactions

Competition between individual reactions in multiplex PCR may aggravate amplification bias and cause an overall decrease in amplification factor compared with uniplex PCR. The dependence of amplification factor on primer concentration can be used to alleviate PCR bias. The variable levels of signal produced from the different loci amplified in the 192-plex PCR of the previous example, taken with the results from Example 3 that show the effect of primer concentration on amplification factor, further suggest that it may be possible to improve the percentage of PCR reactions that generate sufficient target for use in the INVADER assay by modulating primer concentrations.

For example, one particular sample analyzed in Example 5 yielded FOZ results, after a 40 minute incubation in the INVADER assay, of 29.54 FAM and 66.98 RED, while another sample gave FOZ results after 40 min of 1.09 and 1.22, respectively, prompting a determination that there was insufficient signal to generate a genotype call. Modulation of primer concentrations, down in the case of the first sample and up in the case of the second, should make it possible to bring the amplification factors of the two samples closer to the same value. It is envisioned that this sort of modulation may be an iterative process, requiring more than one modification to bring the amplification factors sufficiently close to one another to enable most or all loci in a multiplex PCR reaction to be amplified with approximately equivalent efficiency.

### Example 7

### Multiplex Example

In principle, PCR amplification can be carried out in a multiplex format in which multiple loci are amplified in the same tube. In practice, however, this approach can result in highly variable yields of individual amplified products due to PCR bias. This Example describes the optimization of multiplex reaction conditions to minimize amplification bias. Amplification bias is caused by the variable amplification rate among individual reactions which leads to a significant difference in PCR product yields over a large number of cycles. In this Example, PCR target amplification was analyzed across the full range of the reaction and parameters affecting PCR yield were investigated by using the quantitative INVADER assay. From this work, a model describing the dependence of the target amplification factor on primer concentration and primer annealing time was developed that elucidates a mechanism underlying amplification bias. Using 6-plex PCR as a model system to test different conditions minimizing bias, two approaches were identified. The first relies on adjusting primer concentrations to balance the amplification factors of different loci. In the second approach, the primer concentration was kept the same for all the individual reactions, but the primer annealing time and the number of amplification cycles were optimized to minimize amplification bias. The optimized PCR conditions were used to carry out a 192-plex PCR amplification of 8 genomic DNA samples and for use in genotyping using INVADER assays.

Materials and Methods

Materials. Chemicals and buffers were from Fisher Scientific unless otherwise noted. Structure-specific 5′ nuclease Cleavase enzyme (Third Wave Technologies) was purified as described (5). The enzyme was dialyzed and stored in 50% glycerol, 20 mM Tris HCl, pH 8, 50 mM KCl, 0.5% Tween 20, 0.5% Nonidet P40, 100 μg/ml BSA. Unless otherwise noted, A, G, C and T refer to deoxyribonucleotides.

Preparation of genomic DNA. Eight genomic DNA samples G1, G2, G3, G4, G5, G6, G7 and G8 were prepared from 10 ml of leukocytes using an AutoPure LS instrument (Gentra Systems, Minneapolis, Minn.). The purified DNA was diluted to 13.3 ng/μl in Te buffer containing 10 mM Tris HCl pH 8.0, 0.1 mM EDTA.

Oligonucleotide synthesis. Oligonucleotides used in the INVADER assay with the monoplex and 6-plex PCR reactions were synthesized using a PerSeptive Biosystems instrument and standard phosphoramidite chemistries including A, G, C, T, 6-carboxyfluorescein dye (FAM) (Glen Research), Redmond RED™ dye (RED) (Epoch Biosciences, Redmond, Wash.), and Eclipse™ Dark Quencher (Z) (Epoch Biosciences). The primary probes and FRET cassettes were purified by ion exchange HPLC using a Resource Q column (Amersham-Pharmacia Biotech, Newark, N.J.), and the invasive probes were purified by desalting over NAP-10 columns (Amersham 17-0854-O₂). The primary probes used in the 192-plex PCR assays were synthesized by Biosearch Technologies using C16 CPG columns (Biosearch Technologies, Novato, Calif., BG1-SD14-1), and purified using SuperPure Plus Purification columns (Biosearch, SP-2000-96). The invasive probes for the 192-plex assays were synthesized and purified by Biosearch Technologies using trityl-on 5′ capture purification. PCR primers were synthesized by Integrated DNA Technologies, Chicago, Ill. Oligonucleotide concentrations were determined using the absorption at 260 nm (A₂₆₀) and extinction coefficients of 15,400, 7,400, 11,500, and 8,700 A₂₆₀ M⁻¹ for A, C, G, and T, respectively.

Primer design for multiplex PCR. A computer program, PrimerDesigner software (Third Wave Technologies; Madison, Wis., See FIG. 8 and discussion of Primer Design above), has been developed to assist in designing primers for multiplexed PCR and to reduce the probability of primer-dimer formation. PCR primers for the multiplex format were designed with the PrimerDesigner software using the following parameters in conjunction with the primer design discussion above and in FIG. 8. For each of the loci to be amplified, 500 nucleotides were included on either side of the SNP for a total of 1001 bases per locus. For each locus, the sequence of 60-80 nucleotides required for binding of the invasive and primary probes was determined and candidate forward and reverse PCR primers were identified by “walking” outward from this region. Candidate primers were chosen based on the following criteria: (1) primers must have an A or C at the 3′ end to avoid primer-dimer formation; (2) Tₘ of the primers was 60° C. (11,12); (3) primers should be between 12 and 30 nucleotides in length; (4) the two and three 3′ terminal bases of any primer should not be complementary to the two and three 3′ terminal bases of any other primer of the multiplex PCR mixture; (5) no primer should have more than 80% sequence similarity to the cleaved 5′ arm sequence of either INVADER primary probe. The algorithm is initiated by the design of the first two primers for a randomly selected locus and proceeds by iteration adding more primers to the pool. If no primers can be designed for one of the loci, the algorithm starts from the beginning using a new randomly selected locus.

INVADER assay design. The primary and invasive probes for the INVADER assays were designed with the INVADERCreator algorithm as described elsewhere (Lyamichev, V. and Neri, B. (2003) INVADER assay for SNP genotyping. Methods Mol Biol, 212, 229-40, herein incorporated by reference). The probe sequences for INVADER assays 1-6 corresponding to the PCRs 1-6, respectively. Sequences for the 192 INVADER assays for 192-plex PCR experiments were designed using the same algorithm.

Quantitative analysis of PCR with the INVADER assay. PCRs 1-6 in uniplex or 6-plex format were carried out in 50 μl GeneAmp PCR buffer (PE Biosystems, Foster City, Calif.) containing primers at concentration specified in the text, 0.2 mM dNTPs, 1 μl (5 U/μl) Amplitaq DNA polymerase (PE Biosystems, N₈O₈-0171), 1 μl (1.1 μg/μl) TaqStart Antibody (Clontech, catalog number 5400-2, Palo Alto, Calif.) and 50 ng of human genomic DNA or 3.8 μl Te buffer for the no target control. To prevent evaporation, each well was covered with 15 μl of clear Chill-out (MJ Research, catalog number CHO-1411 Las Vegas, Nev.) and the plates were covered with a foil seal (Beckman Coulter, catalog number BK 538619, Fullerton, Calif.). The number of cycles and time-temperature profile for each cycle are specified in the text. Each PCR included an initial sample denaturing step of 15 min at 95° C. and a final incubation step of 10 min at 99° C. Each reaction was performed in triplicate in a 96-well plate. The PCR products were serially diluted 20-fold in the first step followed by 5-fold subsequent dilution in Te buffer containing 30 μg/ml tRNA (Boehringer Mannheim, cat. no. BK 538619, Indianapolis, Ind.) to bring the product concentrations within the dynamic range of the INVADER assay.

INVADER reactions with the diluted PCR products were carried out in 15 μL containing 0.05 μM invasive oligonucleotide, 0.5 μM of each primary probe, 0.33 μM of each FRET cassette, 5.3 ng/μL Cleavase XI enzyme, 12 mM MOPS (pH 7.5), 15.3 mM MgCl₂, 2.5% PEG 8000, 0.02% NP40, 0.02% Tween 20 overlaid with 15 μl mineral oil (Sigma) in 96-well plate. The PCR products constituted 7.5 μL of the 15 μL reactions. For no-target controls 7.5 μL of Te buffer was used instead of the PCR product. The reactions were incubated at 95° C. for 5 min to denature the target and then at 63° C. for a period of time from 20 min to 3 h. The reactions were stopped by cooling the plates to room temperature, and fluorescence signal was detected with a Cytofluor 4000 fluorescence plate reader (PE Biosystems) using 485/20 nm excitation and 530/25 nm emission filters for the FAM dye and 560/20 nm excitation and 620/40 emission filters for the RED dye. Each PCR replicate was analyzed with the corresponding INVADER assay in triplicate, therefore for each PCR reaction, nine data points were collected.

To determine the concentration of PCR products, standard curves were obtained for each of the INVADER assays 1-6 using standard concentrations of the corresponding PCR products. The PCR standards for the assays 1-6 were prepared by PCR amplification of DNA samples G1, G2, G6, or G8. The amplified products were concentrated by ethanol precipitation, purified using electrophoresis in 8% polyacrylamide non-denaturing gel and quantitated using a Picogreen dsDNA quantitation kit (Molecular Probes, Eugene, Oreg., catalog no. P7589). The INVADER reactions for the standard curves were carried out with 0 to 100 fM of the PCR standards in duplicate in the same microtiter plate as the analyzed PCR products.

The concentration of the analyzed PCR products was determined from the fluorescence signal by a linear regression using the three data points of the standard curve closest to the value of the fluorescence signal of the PCR samples. The PCR product concentration and the variance were estimated for each of the PCR replicates from the triplicate INVADER assay measurements. PCR product concentration for the triplicate PCRs was estimated by using the average values for each of the replicates weighted by the variance of the triplicate INVADER assay analysis. The initial concentration of the genomic DNA samples used in the PCR was determined by the triplicate INVADER assay using the same standard curves. The amplification factor F was determined as the estimated PCR product concentration multiplied by the dilution factor and divided by the genomic DNA concentration used for the PCR.

The 192-plex PCR was carried out in a single replicate under the conditions described for PCRs 1-6 for 17 cycles with the DNA samples G1-G8, each primer concentration of 0.2 μM, primer annealing time 1.5 min, primer extension time 2.5 min and the initial sample denaturing step of 2.5 min at 95° C. For the no-target control 192-plex PCRs, Te buffer was used instead of genomic DNA. The 192-plex PCR reactions were diluted 30-fold in Te buffer containing 30 μg/ml tRNA (Boehringer Mannheim, 109 525) and heated at 95° C. for 5 min prior to addition to the INVADER reactions. The INVADER reactions were performed as described for assays 1-6 except that the invasive probe was at 0.07 μM, and each primary probe was at 0.7 μM. The FAM and RED fluorescence signals were collected after 15, 30 and 60 min or as specified in the text for the genomic PCR samples and no-target PCR controls. The net fluorescence signal was determined by subtracting the no-target signal from the sample signal for each of the 192 INVADER assays. The following algorithm was applied to the analysis by the genotyping software. (1) Fold-over-zero values for the FAM (FOZF) and RED (FOZR) signals were determined for each INVADER assay by dividing the sample signal by the no-target control signal. (2) For each INVADER assay, a ratio value H was determined as (FOZF-1)/(FOZR-1). (3) A sample was defined as heterozygous (HET) if 0.25<H<4 and both FOZF and FOZR>1.3; a sample was defined as homozygous FAM if H>4 and FOZF>1.6; and a sample was defined as homozygous RED if H<0.25 and FOZR>1.6 (4). In all other cases a sample was called an “equivocal”.

To investigate parameters affecting PCR, a method was developed to use the quantitative INVADER assay to determine the target amplification factor F over the full range of the reaction. The F factor was defined as a ratio of concentrations of the amplified product and the initial genomic DNA, both measured with the INVADER assay using standard curves obtained with known amounts of the PCR products as described in “Materials and Methods”.

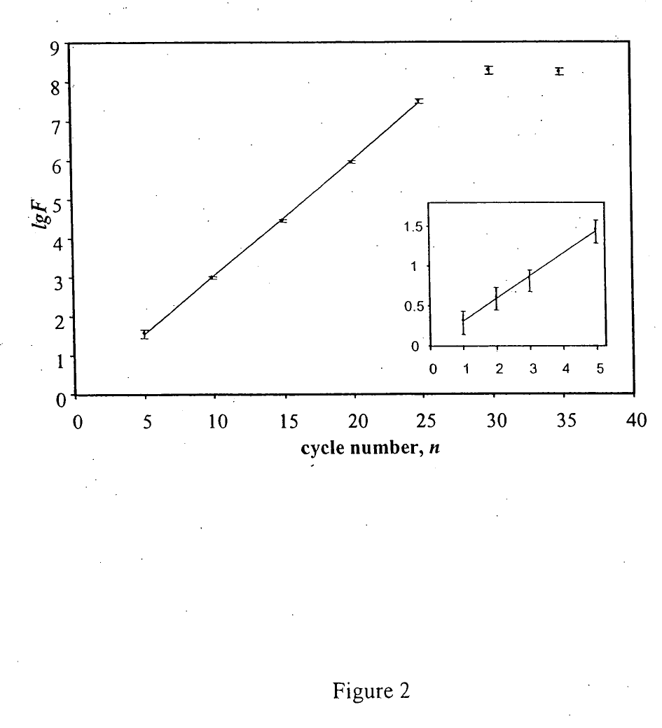

First, F was analyzed as a function of the number of PCR cycles n. The uniplex PCR 5 was performed with a primer concentration c of 0.1 μM using DNA G2, and F was determined after n of 5, 10, 15, 20, 25, 30 and 35 (FIG. 2). As shown in FIG. 2, PCR 5 reveals a linear dependence of lgF on n for the first 25 cycles with a slope of 0.296±0.0016, demonstrating that target amplification is exponential over 7 orders of magnitude. The average amplification factor per PCR cycle determined from slope of the linear dependence is equal to 1.98±0.007, indicating that the amount of the target almost doubles after each cycle. The inset in FIG. 2 shows the dependence of lgF on n for cycles 1, 2, 3, and 5 of PCR 5 under the same conditions except a larger amount of DNA G2 is used as a target. This dependence can also be approximated by a linear function with the lgF vs n slope of 0.283. After 25 cycles, PCR 5 reaches a plateau at F of 2×10⁸ that corresponds to a target concentration of 0.06 μM as determined from the initial genomic DNA concentration of 0.28 fM. The plateau could be explained by either a depletion of the primers used in the PCR at a concentration of 0.1 μM or by an inhibition of the PCR by its own product. Similar to PCR 5, quantitative analysis of PCR 2 shows a linear dependence of lgF on n for the first 25 cycles with a slope of 0.295±0.004 and a plateau at F of 3×10⁸ (data not shown). These results establish the INVADER assay as a quantitative method for PCR target amplification analysis and demonstrate that PCR proceeds exponentially over 7 orders of magnitude or for at least 25 cycles.

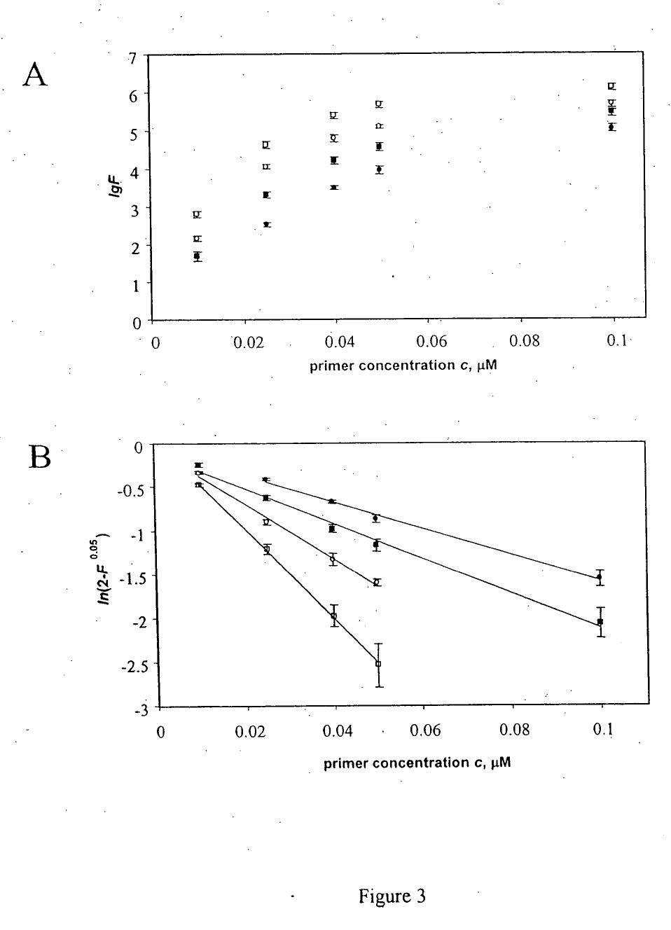

To investigate the effect of c on F as a means of adjusting F and thereby reducing amplification bias (Henegariu, O., et al., Biotechniques, 23, 504-11, 1997) uniplex PCRs 1-6 were investigated using the quantitative INVADER assay. Each PCR was performed for 20 cycles with c of 0.01, 0.025, 0.04, 0.05 or 0.1 μM. The logarithm of F as a function of c is shown for PCRs 1, 2, 4 and 5 in FIG. 3A. FIG. 3A shows the effect of primer concentration c on IgF for the PCR 1 (●), PCR 2(◯), PCR 4 (▪), and PCR 5 (□). PCR amplification was performed in 50 mL with c of 0.01, 0.025, 0.04, 0.05 or 0.1 mM and with 50 ng of the genomic DNA G2 for the PCRs 1, 4, and 5 or the genomic DNA G6 for the PCR 2. Each PCR was performed for 20 cycles using template denaturing step for 30 s at 95° C., primer annealing step for 44 s at 55° C. and primer extension step for 60 s at 72° C. for each cycle. The IgF value for the PCR 1 with c of 0.01 mM was too low for reliable measurements. The standard error was estimated by performing the PCRs in triplicate and by analyzing each replicate by the corresponding quantitative INVADER assay also in triplicate. PCRs 3 and 6 performed very similarly to PCRs 5 and 2, respectively, and are not shown for brevity. There is a significant difference in F between PCRs performed under the same reaction conditions. The difference is most pronounced at low c; however it becomes less significant at higher c where lgF approaches the theoretical maximal value of lg(2²⁰) or 6.0. As shown in the previous section, PCR can be considered to be an exponential reaction at 20 cycles, and F can be used to determine the target amplification factor z in a single PCR cycle as

$${F}^{\frac{1}{n}} .$$

As described previously above, the observed effect of c on F can be described by a model which assumes that primer annealing is the rate limiting step of PCR at lower c. In this model, the binding of primer P to target T is described by a second order reaction with the association rate constant kₐ

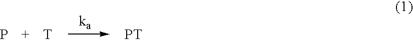

Assuming that the primer is in excess over the target and that the annealing occurs at temperatures below primer melting so that the reverse reaction can be ignored, the solution for the reaction (1) is
[PT]=T₀(1−e^{-k}^{ₐ}^{ct}^{ₐ}) (2)
where [PT] is the concentration of the primed targets, T₀ is the target concentration after the previous PCR cycle, and tₐ is the primer annealing time. Assuming that both PCR primers have the same kₐ. and the annealing time tₐ is long enough to complete duplication of each primed target molecule, z can be determined as

$$z = \frac{{T}_{0} + [ PT ]}{{T}_{0}} = 2 - {ⅇ}^{- {k}_{a} {ct}_{a}} \qquad ( 3 )$$

and F after n cycles is given by
F=zⁿ=(2−e^{-k}^{ₐ}^{ct}^{ₐ})ⁿ (4)
According to Eq 4, ln

$$( 2 - {F}^{\frac{1}{n}} )$$

should be a linear function of c with a slope equal to −kₐtₐ.

Transformation of the data shown in FIG. 3A using ln

$$( 2 - {F}^{\frac{1}{n}} )$$

vs. c coordinates demonstrates the expected linear dependence for each of the PCRs (FIG. 3B) providing a strong support for the model. In FIG. 3B, the straight lines show the least-squares fit for each of the PCRs. The data points for the PCRs 2 and 5 at c of 0.1 mM were not used because of high standard error. The slope of ln

$$( 2 - {F}^{\frac{1}{n}} )$$

vs. c can be used to determine an apparent association rate constant kₐ^{app} of the primer annealing step which is mostly defined by the primer with the lowest kₐ. The kₐ^{app} values for PCRs 1, 2, 4 and 5 determined from FIG. 3B using tₐ of 44 s are 0.34 10⁶, 0.73 10⁶, 0.45 10⁶, and 1.2 10⁶ s⁻¹ M⁻¹, respectively. These values are close to the kₐ values of 1.5 10⁶ s⁻¹ M⁻¹ and 2.6 10⁶ s⁻¹ M⁻¹ obtained for short oligonucleotides under similar buffer conditions. There is at least a three-fold difference between the kₐ for the slowest (PCR 1) and the fastest (PCR 5) suggesting that the primer annealing kinetics can significantly contribute to amplification bias.

The results of the quantitative analysis of PCR amplification suggest two approaches for balanced target amplification in multiplex PCR: (1) adjustment of c for each individual using the dependence of lgF vs. c and (2) increasing c and ta to approach the maximal amplification for all targets at fixed c as follows from Eq. 4.

The adjusted primer concentrations c_{adj} that provide an expected F value of 10⁴ for each of the PCRs 1-6 (Table 1) were determined from the data shown in FIG. 3A.

TABLE 1
Logarithm of the amplification factor lgF for the multiplexed PCRs 1,
2, 3, 4, 5, and 6 under conditions of adjusted primer concentrations.

| | PCR^{a} | | | | | | | | | | | |
|---|---|---|---|---|---|---|---|---|---|---|---|---|
| | 1 | | 2 | | 3 | | 4 | | 5 | | 6 | |
| c_{adj}^{b}, μM | 0.05 | | 0.024 | | 0.017 | | 0.035 | | 0.019 | | 0.029 | |
| lgF_{adj}^{c} | 3.86 | +0.05 | 4.25 | 4.25 | 4.30 | +0.04 | 4.07 | +0.04 | 4.16 | +0.06 | 4.27 | +0.06 |
| | | −0.05 | | −0.05 | | −0.05 | | −0.05 | | −0.06 | | −0.07 |
| c_{0.025}, μM | 0.025 | | 0.025 | | 0.025 | | 0.025 | | 0.025 | | 0.025 | |
| lgF_{0.025}^{c} | 2.40 | +0.10 | 4.23 | +0.04 | 4.82 | +0.05 | 3.26 | +0.05 | 4.62 | +0.04 | 3.98 | +0.08 |
| | | −0.14 | | −0.05 | | −0.05 | | −0.06 | | −0.05 | | −0.09 |

^{a}Multiplexed PCRs 1, 2, 3, 4, 5, and 6 were performed in 50 μL with 50 ng of the genomic DNA G2 or G6 for 20 cycles using denaturing step for 30 s at 95° C., primer annealing step for 44 s at 55° C. and primer extension step for 60 s at 72° C. for each cycle.
^{b}c_{adj} was determined for each of the PCRs 1, 2, 3, 4, 5, and 6 from FIG. 2 to provide expected lgF value of 4.
^{c}lgF_{adj} and lgF_{0.025} for the PCRs 1, 3, 4, 5 and the PCRs 2, 6 were determined using FAM signal of the quantitative INVADER assays and the 6-plex PCRs performed with genomic DNAs G2 and G6, respectively. The standard error was determined from triplicate PCR reactions each analyzed by the corresponding INVADER assay also in triplicate.

The 6-plex PCRs 1-6 were performed with either the adjusted concentrations C_{adj} or a fixed c_{0.025} of 0.025 μM for each of the PCRs under the same conditions as in FIG. 3 using as a target DNAs G2 or G6. As shown in Table 1, under the c_{adj} conditions, all six targets were amplified approximately 10⁴-fold with an average lgF of 4.15±0.17 and a 2.75-fold difference in F between the fastest (PCR 3) and the slowest (PCR 1). Under the c_{0.025} conditions, the amount of the total product amplified in the multiplex PCR was similar to the PCR with C_{adj} with an average lgF of 3.89±0.91, however there was a significant amplification bias as illustrated by a 26.3-fold difference in F between the fastest and the slowest PCRs (3 and 1, respectively). PCRs 1-6 were also carried out in a uniplex format with c_{adj} or c_{0.025} under the conditions of the 6-plex format and demonstrated F values very similar to the corresponding F values shown in Table 1. This result suggests that there is no significant interference between the individual PCRs in the 6-plex format.

Balancing PCR by adjusting c is a powerful approach minimizing the amplification bias; however it uses a known dependence of F on c for each of the PCRs or an iterative optimization of primer concentration. An alternative approach is to use a fixed c value, but to perform PCR under conditions minimizing the bias. Both the experimental data (FIG. 3) and the theoretical analysis (Eq. 3) suggest that z should asymptotically approach 2 as value of the ctₐ term increases. Therefore, multiplex PCRs were performed with a fixed c of 0.1 μM, the maximal concentration used under the conditions shown in FIG. 3, or 0.2 μM and the primer annealing step of 90 s instead of 44 s. The 6-plex PCRs 1-6 were carried out for 17 cycles to provide the theoretically maximal lgF value of 5.1 using as a target the DNAs G1, G2, G6, or G8. Quantitative analysis of F with INVADER assays 1-6 was performed by using both FAM and RED signals (for the genotypes of the genomic DNAs see Table S3) and the lgF values are shown in Table 2.

TABLE 2
Logarithm of the amplification factor lgF for the multiplexed PCRs 1,
2, 3, 4, 5, and 6 under conditions of fixed primer concentrations.

| | | G1 | | | | G2 | | | | G6 | | | | G8 | | | |
|---|---|---|---|---|---|---|---|---|---|---|---|---|---|---|---|---|---|
| PCR^{a} | Dye^{b} | 0.1 μM | | 0.2 μM | | 0.1 μM | | 0.2 μM | | 0.1 μM | | 0.2 μM | | 0.1 μM | | 0.2 μM | |
| 1 | FAM | 4.20 | +0.08 | 4.54 | +0.08 | 4.21 | +0.09 | 4.49 | +0.08 | 4.30 | +0.15 | 4.66 | +0.14 | | | | |
| | | | −0.10 | | −0.09 | | −0.11 | | −0.09 | | −0.22 | | −0.21 | | | | |
| | RED | 4.13 | +0.10 | 4.38 | +0.11 | | | | | 4.26 | +0.11 | 4.57 | +0.15 | 4.27 | +0.14 | 4.65 | +0.14 |
| | | | −0.14 | | −0.14 | | | | | | −0.15 | | −0.24 | | −0.20 | | −0.20 |
| 2 | FAM | | | | | 4.87 | +0.06 | 4.97 | +0.06 | 4.90 | +0.04 | 5.00 | +0.04 | 4.92 | +0.07 | 5.14 | +0.07 |
| | | | | | | | −0.07 | | −0.07 | | −0.04 | | −0.04 | | −0.08 | | −0.08 |
| | RED | 4.83 | +0.08 | 4.93 | +0.08 | 4.86 | +0.06 | 4.94 | +0.06 | | | | | 5.03 | +0.07 | 5.18 | +0.07 |
| | | | −0.10 | | −0.09 | | −0.07 | | −0.07 | | | | | | −0.08 | | −0.08 |
| 3 | FAM | | | | | 4.75 | +0.03 | 4.82 | +0.03 | 4.92 | +0.05 | 4.99 | +0.04 | | | | |
| | | | | | | | −0.03 | | −0.03 | | −0.05 | | −0.05 | | | | |
| | RED | 4.83 | +0.05 | 4.87 | +0.06 | | | | | 4.98 | +0.05 | 5.04 | +0.05 | 5.00 | +0.04 | 5.09 | +0.04 |
| | | | −0.06 | | −0.07 | | | | | | −0.06 | | −0.05 | | −0.04 | | −0.04 |
| 4 | FAM | 4.55 | +0.07 | 4.73 | +0.05 | 4.53 | +0.05 | 4.69 | +0.04 | 4.68 | +0.04 | 4.85 | +0.05 | | | | |
| | | | −0.08 | | −0.06 | | −0.06 | | −0.05 | | −0.05 | | −0.06 | | | | |
| | RED | | | | | | | | | 4.64 | +0.08 | 4.81 | +0.08 | 4.72 | +0.07 | 4.93 | +0.06 |
| | | | | | | | | | | | −0.10 | | −0.09 | | −0.08 | | −0.08 |
| 5 | FAM | 5.19 | +0.09 | 5.24 | +0.10 | 5.25 | +0.08 | 5.32 | +0.09 | | | | | 5.58 | +0.21 | 5.69 | +0.21 |
| | | | −0.11 | | −0.13 | | −0.10 | | −0.11 | | | | | | −0.42 | | −0.43 |
| | RED | | | | | | | | | 5.30 | +0.11 | 5.36 | +0.13 | 5.39 | +0.11 | 5.50 | +0.12 |
| | | | | | | | | | | | −0.15 | | −0.19 | | −0.14 | | −0.17 |
| 6 | FAM | | | | | 5.02 | +0.09 | 5.20 | +0.08 | 5.01 | +0.08 | 5.20 | +0.07 | 5.09 | +0.09 | 5.27 | +0.09 |
| | | | | | | | −0.11 | | −0.10 | | −0.09 | | −0.09 | | −0.11 | | −0.11 |
| | RED | 4.88 | +0.08 | 5.01 | +0.07 | 4.92 | +0.07 | 5.08 | +0.07 | | | | | | | | |
| | | | −0.09 | | −0.08 | | −0.09 | | −0.08 | | | | | | | | |

^{a}Multiplexed PCRs 1, 2, 3, 4, 5, and 6 were performed in 50 μL with c of 0.1 or 0.2 μM, 50 ng of the genomic DNA G1, G2, G6, or G8 for 17 cycles using denaturing step for 30 s at 95° C., primer annealing step for 90 s at 55° C. and primer extension step for 150 s at 72° C. for each cycle. The standard error was determined from triplicate PCR reactions each analyzed by the corresponding INVADER assay also in triplicate.
^{b}Reporting fluorescent dye of the INVADER assay.

The difference between the mean lgF values obtained with the FAM and RED signals was not statistically significant for both the 0.1 and 0.2 μM PCR conditions with the t-test p values of 0.88 and 0.77, respectively, suggesting that the analysis of F was independent of INVADER assay type. The mean lgF values for PCRs 1-6 at c of 0.2 μM were 4.55±0.10, 5.03±0.11, 4.96±0.11, 4.80±0.10, 5.42±0.18 and 5.15±0.11, respectively, or very close to the expected value of 5.1. It is not clear why the lgF value of 5.42 for PCR 5 was statistically higher than expected, although INVADER assay 5 demonstrated a relatively low performance with all of the genomic DNA samples compared to the other assays which may result in an artificially higher ratio of the PCR product and genomic DNA concentrations and overestimated values of lgF.

The difference between the mean lgF values obtained at c of 0.2 and 0.1 μM was 0.32, 0.13, 0.18 and 0.17 for PCRs 1, 2, 4 and 6, respectively. The differences were statistically significant with the corresponding t-test p values of <0.0001, 0.04, 0.01 and 0.02. The difference between the 0.2 and 0.1 μM mean lgF values for the fastest PCRs (3 and 5) was 0.07 and 0.08, respectively, with the t-test p values of 0.37 and 0.47 assuming no statistical significance. This analysis demonstrates that increase of the ctₐ term improves performance of the slower PCRs and does not affect performance of the fast PCRs in the multiplex reaction that have apparently approached the amplification plateau.

The next part of this Example was the development of 192-plex PCR, essentially doubling the multiplex factor of 100 achieved by (Ohnishi, et al., J Hum Genet, 46, 471-7, 2001), for SNP genotyping with the INVADER assay. 192 SNPs representing chromosomes 5, 11, 14, 15, 16, 17 and 19 were randomly selected and an INVADER assay was designed for each of the SNPs. During the selection process, no discrimination against SNPs in repetitive regions was carried out. Therefore some of the 192 SNPs were likely to be amplified at multiple loci. PCR conditions developed for balanced amplification were used with a fixed primer concentration because of simplicity and short development time. Genomic DNA samples G1-G8 were amplified with the 192-plex PCR for 17 cycles with fixed c of 0.2 μM, primer annealing time of 1.5 min, primer extension time of 2.5 min, and then analyzed with the 192 biplex INVADER assays as described in “Materials and Methods”. The RED and FAM net signals were obtained by subtracting the no-target control signal from the sample signal. One way to identify genotypes from the net signals is to use universal calling criteria for each of the assays as described in the “Materials and Methods”. These criteria assume that the homozygous samples have only signal from one of the alleles with no or very little cross-reactivity signal from the other one, and that heterozygous samples produce approximately equal signals for both alleles. Such rigid criteria can often lead to equivocal calls in otherwise functional INVADER assays.

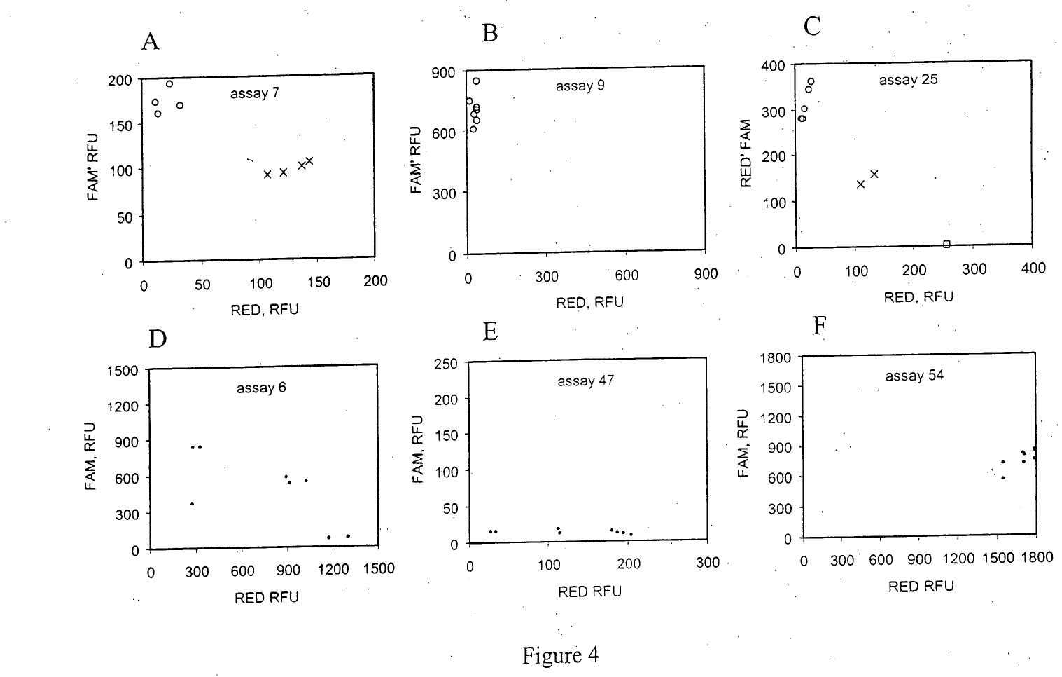

As an alternative, genotypes were called by plotting the FAM and RED net signals for all eight DNA samples as a scatter plot for each of the INVADER assays and visually identifying clusters corresponding to the homozygous and heterozygous samples. Scatter plot analysis cannot be performed if too few samples are included; this analysis also contains an element of subjectivity, since this type of visual analysis depends on the judgment of the operator. In this work, it was determined that eight samples are sufficient to make visual calls for the majority of the 192 INVADER assays. Examples of both successful and failed scatter plot analyses are shown in FIG. 4. FIG. 4 shows scatter plots of the net FAM and RED INVADER assay signals for eight genomic DNA samples. The INVADER assay net FAM and RED signals were plotted for the DNA samples G1, G2, G3, G4, G5, G6, G7 and G8 amplified with the 196-plex PCR. A-C, successful genotyping with the assays 7, 9, and 25 assigning all samples to distinctive clusters identified as homozygous FAM (◯), homozygous RED (□) or heterozygous (x). D-F, failed genotyping. In the assay 6 (D), the sample closest to the origin of coordinates cannot be assigned to any of the clusters; in the assay 47 (E), the samples form three distinctive clusters but there is no FAM signal for any of the samples; in the assay 54 (F), the samples cannot be distinguished between homozygous RED with high FAM signal cross-reactivity and heterozygous with skewed RED/FAM ratio. RFU—relative fluorescence units.

Conservative criteria were used for the visual analysis, excluding a whole set of samples if just one of the samples could not be assigned to a cluster. Also, sets with strong signals in both channels were not considered to give accurate genotypes, assuming a high cross-reactivity of the INVADER assay or, most likely, amplification of multiple homologous loci by the PCR. Using these criteria, calls were made for 161 or 84% of the 192 assays. Calls made using the genotyping software described in the “Materials and Methods” agreed with 82.5% of these calls.

The 31 failed INVADER assays were investigated to determine whether the failure was due to a low PCR amplification factor, poor INVADER assay performance, or amplification of highly homologous sequences by the PCR. The PCR target sequences were analyzed using BLAT to determine if any of the individual PCRs amplified more than one locus. Eight of 31 assays apparently failed because, for each of them, multiple loci were likely amplified by the PCR and each of the loci could be detected by the INVADER assay. The remaining 23 assays were assumed to fail because of one or a combination of the following reasons: poor PCR amplification, flaw in oligonucleotide design and manufacturing, or unrecognized repeat sequences not included in the April 2003 human genome assembly. Excluding the 8 assays that failed because of repeat sequences in the genome, the efficiency of the 192-plex PCR with INVADER assay genotyping was estimated as 161/184 or 87.5%.

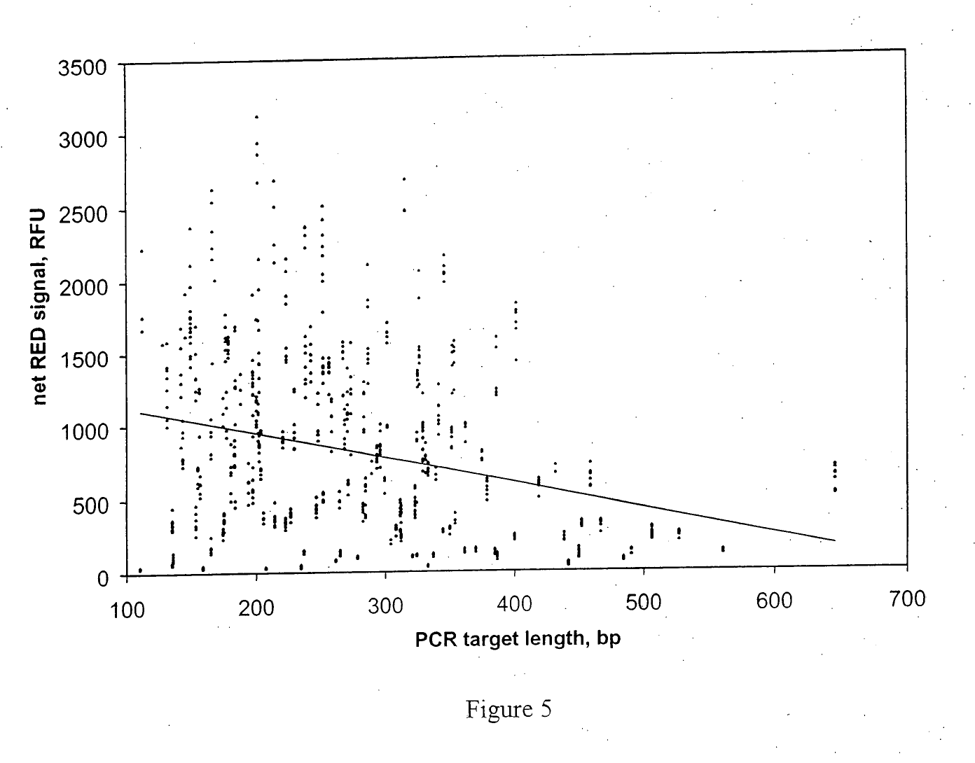

To estimate the amplification bias in the 192-plex PCR, the RED net fluorescence signal normalized per allele was plotted for the 161 successful INVADER assays performed on the eight DNA samples versus PCR target length as shown in FIG. 5. FIG. 5 shows the net RED fluorescence signal normalized per allele for the 161 successful INVADER assays as a function of PCR target length. The INVADER reactions were performed for 60 min with the eight DNA samples each amplified with the 196-plex PCR. The line shows a linear regression of the net signal as a function of PCR target length.

There is significant variability in the net signal that includes variability in PCR amplification and INVADER assay performance. Similar results were obtained for the FAM net signal. There is a weak correlation between the net signal and target length suggesting that PCR targets longer than 700 bp would have low probability of permitting successful genotyping.

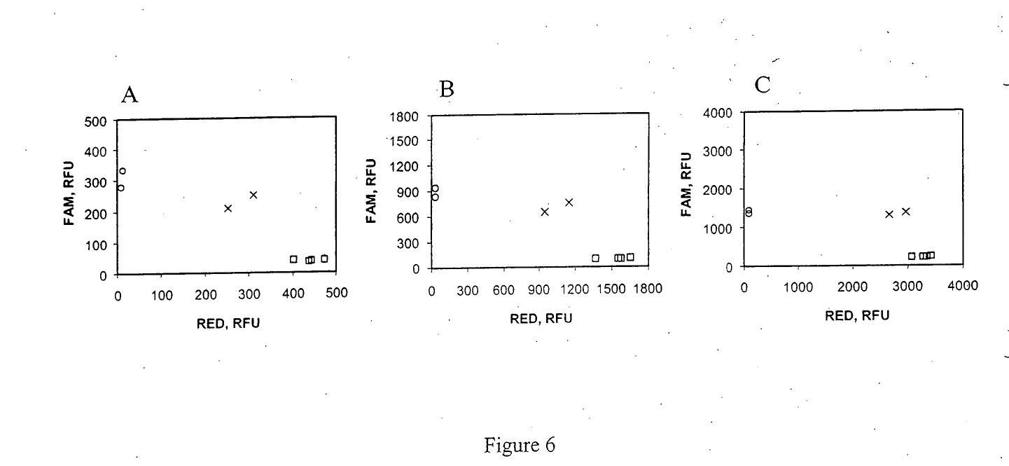

Surprisingly, despite the high variability in the net signal, the genotyping was successfully performed at the both high and low ends of the signal distribution. To investigate the observed robustness of INVADER assay genotyping, the net signal for the same 192 INVADER reactions was measured after 15, 30 and 60 min. Because signal amplification in the INVADER assay is quadratic with time (1), the 30 and 60 min time points would be equivalent to the 15 min reaction performed with the 4-fold and 16-fold higher target level, respectively, thus modeling low, intermediate and high levels of PCR amplification. As an example, scatter plots for INVADER assay 110 obtained at 15, 30 and 60 min time points are shown in FIG. 6. FIG. 6 shows scatter plots of the net FAM and RED signals for the eight DNA samples. The INVADER assay 110 was performed with the DNA samples amplified with 196-plex PCR and signal was measured after 15 (A), 30 (B) and 60 min (C). The samples were identified as homozygous FAM (◯), homozygous RED (□) or heterozygous (x) by the scatter plot analysis. RFU—relative fluorescence units.

The scatter plots demonstrate that INVADER genotyping by cluster analysis is not affected by a strong net signal and can be interpreted even for the 60 min reaction, where both the FAM and RED net signal reach saturation. As a result of this effect, more calls can be made with longer INVADER reactions, because more signal is generated for slow PCRs, improving genotype identification, but at the same time the higher signal for the fast PCRs does not affect sample clustering.

### Example 8

### PCR Amplification and Invader Assay Analysis in a Single Reaction Vessel

This example describes a method for using PCR to amplify small amounts of a target followed by INVADER assay analysis is a single reaction vessel. In particular, this example describes conducting these two reactions without the need for manipulations or reagent additions after a single reaction set-up. Unless otherwise stated, the following examples were carried out with the indicated reagents for assays to detect sequences in the DLEU gene (chromosome 13) and α-actin gene (chromosome 1):
10 mM MOPS buffer, ph 7.5
7.5 mM MgCl₂
dNTPs, 25 μM each
genomic DNA 10 ng
PCR primers 200 nM each
Primary probes 0.5 pM
INVADER oligos 0.05 μM
FRET probes 0.05 μM
CLEAVASE enzyme (VIII or X) 100 ng
Stoffel or native Taq DNA polymerase 1 u

PCR Primers for DLEU:

Forward primer 1716-14-1:

(SEQ ID NO: 1)

5′-CCCGACATTTTTACGCATGCGCAAACTCCAACC-3′,
Tm = 73.8° C.
Reverse primer 1716-14-2:

(SEQ ID NO: 2)

5′-TACACGCACGCGCAAGAAGCAAGAGGACT-3′,
Tm = 74.1° C.

PCR Primers for α-Actin:

Forward primer 1716-14-3:

(SEQ ID NO: 3)

5′-CTGGGTTTCCAACAGGCGAAAAGGCCCT-3′, Tm = 73.4° C.
Reverse primer 1716-14-4:

(SEQ ID NO: 4)

5′-GCGTGAGGGTGGAAGGAGATGCCCATGG-3′, Tm = 74.7° C.

Probes, INVADER oligos, FRET cassettes (underlined bases indicate flap sequences; bold bases indicate position 1 in the INVADER assay)
α-actin probe 1734-57 ACGGACGCGGAGAGGAACCCTGTGACAT-hex (SEQ ID NO: 5)
a-actin INVADER oligo 1734-57 CCATCCAGGGAAGAGTGGCCTGTTT (SEQ ID NO: 6)
DLEU probe CGCGCCGAGGTTCTGCGCATGTGC-hex (SEQ ID NO: 7)
DLEU INVADER oligo AGGGAGAGCCGTGCACCACGATGAC (SEQ ID NO: 8)
DLEU FAM FRET 23-428 Fam-TCT-Z28-AGCCGGTTTTCCGGCTGAGACCTCGGCGCG-hex (SEQ ID NO: 9)
α-actin RED FRET Red-TCT-Z28-TCGGCCTTTTGGCCGAGAGACTCCGCGTCCGT-hex (SEQ ID NO: 10).
   A. Configuration of Combined PCR-INVADER Reactions

In some cases, it may be desirable to separate the PCR and INVADER reactions temporally, e.g. by carrying out the PCR reaction under conditions that disfavor the INVADER reaction and then modifying the reaction conditions to permit the INVADER reaction to proceed. One such means of creating differential reaction conditions is via the use of antibodies to the enzymes used in the reaction, such as the Light Cycler TaqBlock antibody (Roche Applied Sciences). Another such means is via temperature. In present example, PCR primers were designed with annealing temperatures ≧70° C. while the probe oligonucleotides for use in the INVADER assay were designed with Tm of approximately 63° C., such that the probes should not be capable of reacting with target molecules during the annealing, extension, or denaturation phases of the PCR cycle. In addition, it was determined that while both Stoffel fragment of Taq DNA polymerase and native Taq DNA polymerase can be inactivated by prolonged exposure to elevated temperature (in this case, 99° C. for 10 minutes), some CLEAVASE enzymes retain activity following such treatment. In particular, CLEAVASE VIII appears to be highly stable to such heating and was used in subsequent experiments.

Reactions were carried out in which all reagents were combined in a final volume of 10 μl using the components described above and overlaid with mineral oil. PCR was allowed to proceed for 11-20 cycles (95° C. for 30 seconds; 72° C. for 30 seconds to 2 minutes). Following these cycling reactions, mixtures were heated to 99° C. for 10 minutes to inactivate the Taq DNA polymerase. The reaction mixtures were then incubated at 63° C. for 30 minutes to 3 hours to allow the INVADER reactions to proceed.

B. Evaluation of Inhibition of INVADER Assay Signal Generation

Initial results indicated that there appeared to be inhibition limiting the signal generation of the INVADER assay. The following experiments were conducted to evaluate the possible contribution of various reaction components to this inhibition.

Partial reactions were assembled in order to examine the effects of various reaction components. Specifically, various INVADER reaction components were omitted from the initial reaction set up and then added to the reactions following thermal inactivation of the DNA polymerase. In the following tables, “+” indicates that a component was included in the initial reaction set up; “−” indicates that a component was added following thermal denaturation of Taq DNA polymerase in order to allow INVADER reactions to proceed.

| | | | 1 | 2 | 3 | 4 | 5 | 6 | 7 | 8 | 9 | 10 | 11 | 12 |
|---|---|---|---|---|---|---|---|---|---|---|---|---|---|---|
| Mops 100 mM | 1 | ul | + | + | + | + | + | + | + | + | + | + | + | + |
| MgCl2 | 1 | ul | + | + | + | + | + | + | + | + | + | + | + | + |
| dNTP 1.25 mM ea | 0.2 | ul | + | + | + | + | + | + | + | + | + | + | + | + |
| Primers 1716-14-1/2 5 uM | 0.4 | ul | + | + | + | + | + | + | + | + | + | + | + | + |
| ea | | | | | | | | | | | | | | |
| Primers 1716-14-3/4 5 uM | 0.4 | ul | + | + | + | + | + | + | + | + | + | + | + | + |
| ea | | | | | | | | | | | | | | |
| Stoffel 10 u/ul | 0.1 | ul | + | + | + | + | + | + | + | + | + | + | + | + |
| gDNA 03-422 100 ng/ul | 1 | ul | + | + | + | + | + | + | − | − | − | − | − | − |
| Dleu/a-actin PPI-FRET 5X (-Dleu Inv) | 3 | ul | − | + | − | − | + | − | − | + | − | − | + | − |
| Invader Dleu 2.5 uM | 0.6 | uM | − | − | − | + | + | + | − | − | − | + | + | + |
| Cleavase VIII 100 ng/ul | 1 | ul | − | − | + | − | + | + | − | − | + | − | + | + |

| | |
|---|---|
| Volume 10 ul | (95 C. 30″ −> 72 C. 30″)20 −> 98 C. 5′ |

After PCR add missing components (−) in 5 ul 10 mM Mops
7.5 mM MgCl2 and run 95 C. 3′ −> 63 C. 3 h

| | | | | | | | | | | | | | |
|---|---|---|---|---|---|---|---|---|---|---|---|---|---|
| Ex: | 485/20 | 1478 | 80 | 1158 | 1519 | 67 | 1250 | 88 | 61 | 53 | 68 | 68 | 55 |
| Em: | 530/25 | | | | | | | | | | | | |
| Gain: | 45 | | | | | | | | | | | | |
| Ex: | 560/20 | 1977 | 1233 | 1810 | 2039 | 512 | 1860 | 70 | 78 | 70 | 87 | 98 | 57 |
| Em: | 620/40 | | | | | | | | | | | | |
| Gain: | 50 | | | | | | | | | | | | |

Comparison of the results in columns 2 and 5, in which the FRET mixes were included during the PCR reaction, to those in columns 1, 3-4 and 6, in which FRET probes were not added until after the PCR reaction had been arrested suggests that signal generated in the INVADER assay is inhibited by the presence of the PPI-FRET mixes. Subsequent experiments (see below) in which each component of the PPI-FRET mixes was omitted during the PCR reaction confirms that the FRET probes were inhibitory.

| | | | | | | | | | | |
|---|---|---|---|---|---|---|---|---|---|---|
| Mops 100 mM | 1 | ul | + | + | + | + | + | + | + | + |
| MgCl2 | 1 | ul | + | + | + | + | + | + | + | + |
| dNTP 1.25 mM ea | 0.2 | ul | + | + | + | + | + | + | + | + |
| Primers 1716-14-1/2 5 uM ea | 0.4 | ul | + | + | + | + | + | + | + | + |
| Primers 1716-14-3/4 5 uM ea | 0.4 | ul | + | + | + | + | + | + | + | + |
| Stoffel 10 u/ul | 0.1 | ul | + | + | + | + | + | + | + | + |
| gDNA 03-422 100 ng/ul | 1 | ul | + | + | + | + | + | + | + | + |
| Dleu probe 15 uM | 0.5 | ul | − | + | − | − | − | − | − | + |
| a-actin probe 15 uM | 0.5 | ul | − | − | + | − | − | − | − | + |
| Dleu invader 2.5 uM | 0.5 | ul | − | − | − | + | − | − | − | + |
| a-actin invader 2.5 uM | 0.5 | ul | − | − | − | − | + | − | − | + |
| Dleu FRET 23-428 7.5 uM | 0.5 | ul | − | − | − | − | − | + | − | + |
| a-actin FRET 23-755 7.5 uM | 0.5 | ul | − | − | − | − | − | − | + | + |

| | |
|---|---|
| Volume 10 ul | (95 C. 30″ −> 72 C. 30″)20 −> 98 C. 5′ |

After PCR add missing components (−) in 5 ul 10 mM Mops
7.5 mM MgCl2 and run 95 C. 3′ −> 63 C. 3 h

| | | | | | | | | | |
|---|---|---|---|---|---|---|---|---|---|
| Ex: | 485/20 | 108 | 1782 | 1800 | 1692 | 1720 | 100 | 94 | 89 |
| Em: | 530/25 | | | | | | | | |
| Gain: | 45 | | | | | | | | |
| Ex: | 560/20 | 2363 | 3313 | 3053 | 3243 | 2950 | 2284 | 1706 | 556 |
| Em: | 620/40 | | | | | | | | |
| Gain: | 50 | | | | | | | | |

Examination of the three right-most columns in this table indicates that INVADER assay signal generation was reduced for those reactions in which either or both FRET probes were present (“+”) from the initiation of the reaction relative to those in which it was omitted.

Additional experiments in which the amount of Taq polymerase was increased demonstrated that a 2-fold increase in Stoffel DNA polymerase resulted in increased signal generation in the INVADER assay. Based on these experiments, it was determined that increasing the extension time during the PCR reaction as well as optimizing Taq DNA polymerase concentration reduced the impact of this inhibition.

C. Optimization of Combined PCR and INVADER Assay Reaction Conditions

Experiments were carried out to optimize the amounts of various reaction components and the times of various steps in the combined assays. The concentration of MgCl2 was varied over a range of 1.7 mM to 7.5 mM; dNTP concentrations were tested over a range of 25-75 mM; primer concentration was varied from 0.2 μM-0.4 μM. Exemplary data obtained using native Taq polymerase are presented below and indicate that FAM signal generation is dependent on the presence of the DLEU INVADER oligo and that both INVADER reactions generate signal following 17 cycles of PCR followed by 10 min at 99° C. to denature the native Taq DNA polymerase followed by a 30 minute INVADER reaction at 61° C.

| | | | | | | |
|---|---|---|---|---|---|---|
| Mops 100mM | 1.5 | ul | + | + | + | + |
| MgCl2 25 mM | 1.5 | ul | + | + | + | + |
| Dleu/a-actin PPI-FRET | 3 | ul | + | + | + | + |
| 5X (−Dleu Inv) | | | | | | |
| Dleu Invader 2.5 uM, | 0.3 | ul | + | + | − | + |
| 0.05 uM final | | | | | | |
| Cleavase VIII 100 ng/ul | 1 | ul | + | + | + | − |
| TaqPol (native) 5 u/ul | 0.2 | ul | + | + | + | + |
| Primers 1716-14-1/2 | 1.2 | ul | + | + | + | + |
| 5 uM ea, 0.4 uM final | | | | | | |
| Primers 1716-14-3/4 | 1.2 | ul | + | + | + | + |
| 5 uM ea, 0.4 uM final | | | | | | |
| dNTP 1.25 mM ea, | 0.3 | ul | + | + | + | + |
| 25 uM final | | | | | | |
| gDNA 03-422 10 ng/ul | 1 | ul | − | + | + | + |
| Volume 15 ul | | | | | | |

(95 C. 30″ −> 72 C. 2′)17 −> 99 C. 10 −> 61 C. 30′

| | | | | | |
|---|---|---|---|---|---|
| Ex: | 485/20 | 110 | 1744 | 115 | 87 |
| Em: | 530/20 | | | | |
| Gain: | 45 | | | | |
| Ex: | 560/20 | 123 | 2642 | 2700 | 112 |
| Em: | 620/40 | | | | |
| Gain: | 50 | | | | |

D. Dose Response of the Combined PCR-INVADER Assay

Experiments were carried out to monitor signal generation in the combined PCR-INVADER assay over a range of starting genomic DNA target concentrations. Reactions were set up as follows:

| | | | 1 | 2 | 3 | 4 | 5 | 6 | 7 | 8 |
|---|---|---|---|---|---|---|---|---|---|---|
| Mops 100 mM | 1.5 | ul | + | + | + | + | + | + | + | + |
| MgCl2 75 mM | 0.75 | ul | + | + | + | + | + | + | + | + |
| Dleu/a-actin | 3 | ul | + | + | + | + | + | + | + | + |
| PPI-FRET 5X | | | | | | | | | | |
| Cleavase VIII | 1 | ul | + | + | + | + | + | + | + | + |
| 100 ng/ul | | | | | | | | | | |
| TaqPol (native) | 0.1 | ul | + | + | + | + | + | + | + | + |
| 5 u/ul | | | | | | | | | | |
| Primers 1716- | 0.6 | ul | + | + | + | + | + | + | + | + |
| 14-1/2 5 uM ea, | | | | | | | | | | |
| 0.2 uM fnl | | | | | | | | | | |
| Primers 1716- | 0.6 | ul | + | + | + | + | + | + | + | + |
| 14-3/4 5 uM ea, | | | | | | | | | | |
| 0.2 uM fnl | | | | | | | | | | |
| dNTP 1.25 mM ea, | 0.3 | ul | + | + | + | + | + | + | + | + |
| 25 uM final | | | | | | | | | | |
| gDNA 5 ng | 02948A | | − | 2 | 1.5 | 1 | 0.75 | 0.5 | 0.3 | 0.1 |
| Volume 15 ul | | | | | | | | | | |

(95 C. 30″ −> 72 C. 2′)12 −> 99 C. 10′ −> 60 C. 60′

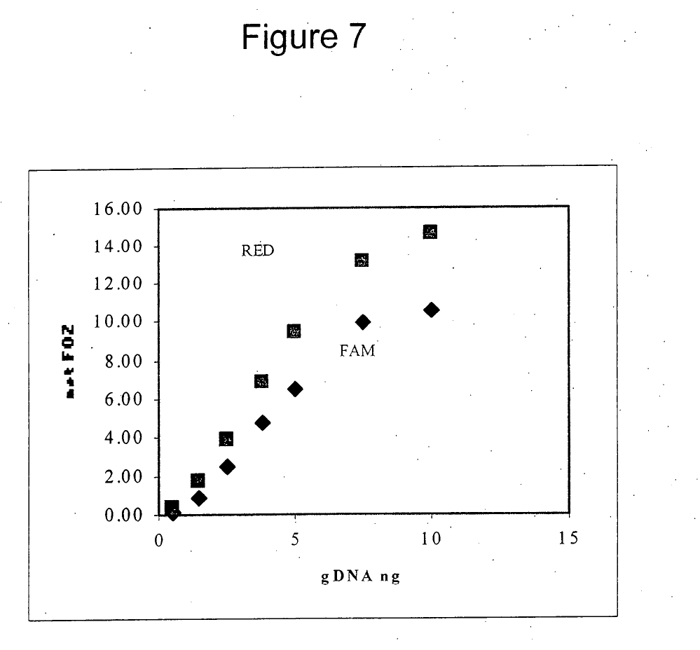

INVADER reactions were allowed to proceed for 120 minutes, and results were read after 60 minutes or 120 minutes. Results from the 120 minute read are presented in FIG. 7. These results indicate that the combined PCR-INVADER reaction is linear over a range of DNA 10 concentrations and is sufficiently sensitive to detect as little as 1.5 ng of human genomic DNA.

E. Multiplex PCR Combined with Biplex INVADER Assay Detection

Additional experiments were conducted to analyze multiplex PCR reactions in combination with the INVADER assay. 20-plex PCR reactions were set up as described below. The PCR CF mix contained each of the primers in the table below at a concentration of 1 μM. Genomic DNA samples were obtained from Coriel as follows in the table below.

| | vol (ul) | 10× vol |
|---|---|---|
| MgCl₂ (50 mM) | 2.25 | 22.5 |
| Cleavase VIII 100 ng/ul | 1.00 | 10.0 |
| Native Taq pol (5 units/ul) | 0.10 | 1.0 |
| dNTP mix 1.25 mM | 0.30 | 3.0 |
| PCR primer mix (1869-80)(1 uM each) | 3.00 | 30.0 |
| gDNA DNA 10 ng/uL | 1.00 | 10.0 |
| H2O | 4.35 | 43.5 |
| sum | 12.00 | 120.00 |

Coriel samples were numbered as follows (e.g. “C” n)

| | Coriell # | Genotype |
|---|---|---|
| 1 | NA11277 | I507 del HET |
| 2 | NA11280 | 711 + 1G > T/621 + 1G > THET |
| 3 | NA01531 | delF508 HOM |
| 4 | NA04539 | delF508 HOM |
| 5 | NA07381 | delF508/3849 + 10 kb HET |
| 6 | NA07441 | 3120 + 1G > A/621 + 1G > THET |
| 7 | NA07469 | delF508/R553X |
| 8 | NA11283 | A455E/delF508 HET |
| 9 | NA11284 | R560T/delF508 HET |
| 10 | NA11286 | delF508/G551D HET |
| 11 | NA11290 | A455E/621 + 1G > THET |
| 12 | NA07552 | delF508/R553X |
| 13 | NA08342 | delF508/G551D HET |
| 14 | NA11275 | 3659delC/delF508 HET |
| 15 | NA12785 | G551D/R347P HET |
| 16 | NA11472 | G1349D/N1303K HET |
| 17 | NA11496 | G542X HOM |
| 18 | NA11497 | G542X HET |
| 19 | NA11723 | W1282X HET |
| 20 | NA11761 | G551D/R553X HET |
| 21 | NA11282 | G85E/621 + 1G > THET |
| 22 | NA12960 | R334W/unknown mutation |
| | | HET |
| 23 | NA13032 | I506V |
| 24 | NA13033 | F508C |
| 25 | NA13423 | G85E/D1152H HET |
| 26 | NA11859 | 2789 + 5G > A HOM |
| 27 | NA11860 | 3849 + 10 kb HOM |
| 28 | NA12444 | 1717 − 1G > A HET |
| 29 | NA12585 | R1162X HET |
| 30 | NA13591 | delF508/R117H HET |
| 31 | NA08338 | G551D |
| 32 | NA11281 | 621 + 1G > T/delF508 |
| 34 | NA12961 | V520F |
| 35 | NA11278 | Q493X/delF508 |
| 36 | NA11285 | Y1092X (C > A)/delF508 |
| 38 | NA00946 | AN1 |
| 39 | NA00130 | AN2 |

PCR primers were selected from the following.

| | |
|---|---|
| cftr exon 3 | TGGTCCCACTTTTTATTCTTTTGCAGA |
| cftr exon 4 | AAGTCACCAAAGCAGTACAGCC |
| cftr exon 5 | GCTGTCAAGCCGTGTTCTAGATAAA |
| cftr exon 7 | CGGAAGGCAGCCTATGTGAGA |
| cftr exon 9 | CATGGGCCATGTGCTTTTCAAAC |
| cftr exon 9-1 | CATGGGCCATGTGCTTTTCAAAC |
| cftr exon 9-2 | CTTCTTGGTACTCCTGTCCTGAAAGA |
| cftr exon 10 | ATTATGGGAGAACTGGAGCCTTCA |
| cftr exon 11 | GATTACATTAGAAGGAAGATGTGCCTTTCAA |
| cftr exon 12 | TAAGGCAAATCATCTACACTAGATGACCA |
| cftr exon 13 | TAACTGAGACCTTACACCGTTTCTCA |
| cftr exon 14B | ATGGGAGGAATAGGTGAAGATGTTAGAA |
| cftr exon 16 | TCTGAATGCGTCTACTGTGATCCA |
| cftr exon 17A | CCTGCACAATGTGCACATGTACC |
| cftr exon 17B | GGACTATGGACACTTCGTGCC |
| cftr exon 18 | GGAGAAGGAAGAGTTGGTATTATCCTGAC |
| cftr exon 19 | GCATCAAACTAATTGTGAAATTGTCTGCC |
| cftr exon 19-1 | GCATCAAACTAATTGTGAAATTGTCTGCC |
| cftr exon 19-2 | GAAGGTGGAAATGCCATATTAGAGAACA |
| cftr exon 20 | GTACCTATATGTCACAGAAGTGATCCCA |
| cftr exon 21 | GATTAGAAAAATGTTCACAAGGGACTCCA |
| cftr 3849 + 10 kb | CAGTTGACTTGTCATCTTGATTTCTGGA |
| cftr exon 17A-2 | CCTCGACAATGTGCACATGTACC |
| | reverse primer |
| cftr exon 3 | ACCTATTCACCAGATTTCGTAGTCTTTTCA |
| cftr exon 4 | TGTACCAGCTCACTACCTAATTTATGACA |
| cftr exon 5 | GAGCTGAGCAAGACTTAACCACTAATTAC |
| cftr exon 7 | GTGAACATTCCTAGTATTAGCTGGCAAC |
| cftr exon 9 | CTCCAAAAATACCTTCCAGCACTACAAA |
| cftr exon 9-1 | GAAATTACTGAAGAAGAGGCTGTCATCAC |
| cftr exon 9-2 | CTCCAAAAATACCTTCCAGCACTACAAA |
| cftr exon 10 | GACTAACCGATTGAATATGGAGCCAAA |
| cftr exon 11 | CTVAAATGTGATTCTTAACCCACTAGCCA |
| cftr exon 12 | GAGGTAAAATGCAATCTATGATGGGACA |
| cftr exon 13 | TAAGGGAGTCTTTTGCACAATGGAAAA |
| cftr exon 14B | ACCTCACCCAACTAATGGTCATCA |
| cftr exon 16 | TAGACAGGACTTCAACCCTCAATCA |
| cftr exon 17A | GAGTATCGCACATTCACTGTCATACC |
| cftr exon 17B | AAGGTAACAGCAATGAAGAAGATGACAAA |
| cftr exon 18 | TAATGACAGATACACAGTGACCCTCAA |
| cftr exon 19 | GCTTCAGGCTACTGGGATTCAC |
| cftr exon 19-1 | GTCATCTTVCTTCACGTGTGAATTCTCAA |
| cftr exon 19-2 | GCTTCAGGCTACTGGGATTCAC |
| cftr exon 20 | TTCTGGCTAAGTCCTTTTGCTCAC |
| cftr exon 21 | CATTTCAGTTAGCAGCCTTACCTCA |
| cftr 3849 + 10 kb | TCCTCCCTGAGAATGTTGGATCAA |
| Vs1 Int std F | TGATGGTGGTATGTTTTCAGGCTAGA |
| Vs1 Int std R | GTTCTCCCCTGTCCCAGTTTTAAC |

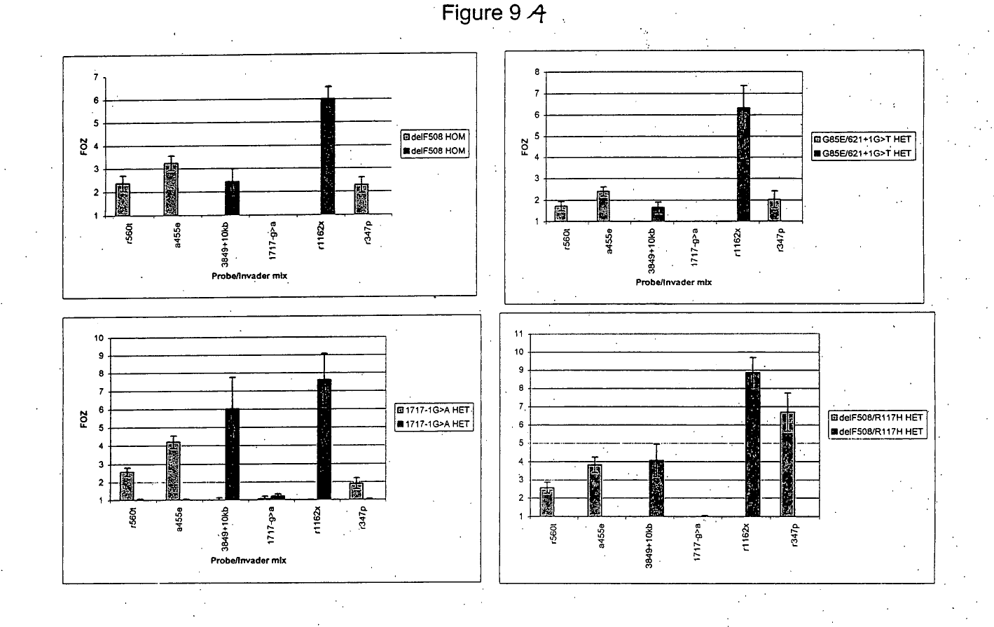

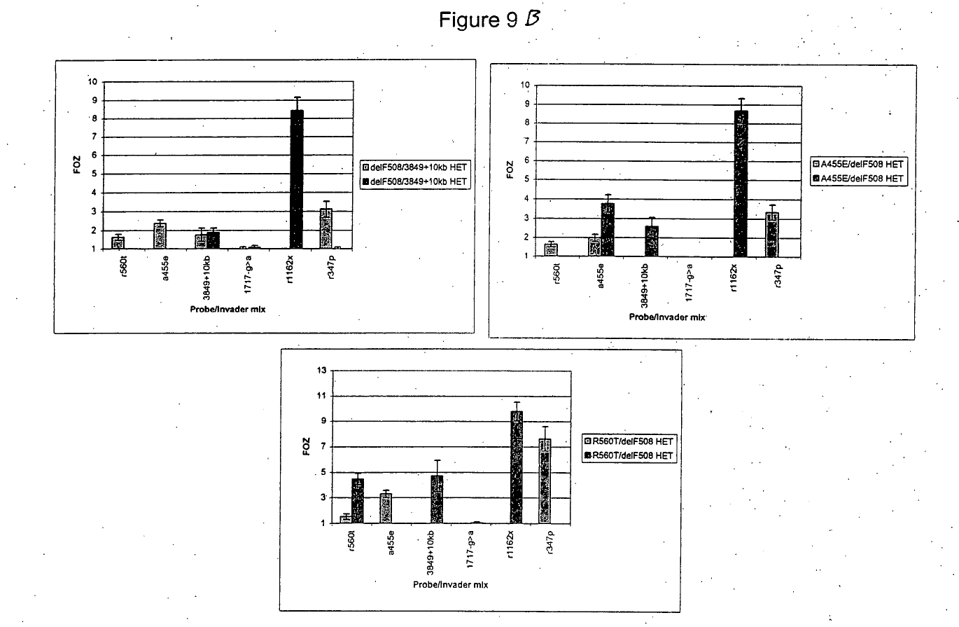

PCR reactions were run as described above with a 2.5 minute extension at 72° C. and a 45 sec denaturation at 95° C. for 14 cycles. Mixtures were heated to 99° C. for 10 minutes and then cooled to 63° C. for 1 hour. The results are presented in FIG. 9. The delF508 sample was Coriel #3; the G85E/621+1 G>T was Coriel 21; 1717-1G>A, Coriel 28; delF508/R117H was Coriel 30; delF508/3849+10 kb, Coriel 5; A455E/delF508, Coriel 8, and R560T/delF508 Coriel 9. These results indicate that the combined PCR plus INVADER assay can be run with multiplex PCR reactions.

### Example 9

### Tetraplex Invader Assay: 4-Dye System

A further means of increasing analysis throughput is to increase the number of INVADER reactions that can be run and analyzed in a single reaction or reaction vessel. The present example describes the implementation of a 4-plex INVADER assay in which four sets of oligonucleotides are included in a single reaction. In this case, the reaction also included four distinct target sequences: wild type and variant versions of two different SNPs. Alternative configurations are also contemplated, including four distinct loci, three distinct loci and one internal control, etc.

One variable in configuring the INVADER assay for multiplex FRET analysis is related to the choice of dyes for inclusion on the FRET probes. Numerous combinations of dyes and quenchers are known in the art (see, e.g., U.S. Pat. Nos. 5,925,517, 5,691,146, and 6,103,476, each incorporated herein by reference). In some embodiments, it is desirable to select dye-quencher combinations that exhibit minimal interference with the cleavage activity of the CLEAVASE enzyme. Such dye-quencher combinations when used with the INVADER assay may favor a more optimal turnover rate.

Another consideration affecting the choice of dyes relates to their spectral characteristics. In some embodiments, e.g., for assays detected in a fluorescence plate reader, it is preferred that the fluorescent signals from each dye be spectrally resolvable from one another by the instrument. If they are not sufficiently spectrally distinct, the fluorescence output from one dye could interfere or “bleed over” into the signal attributed to another dye. This “cross talk” can lead to decreased assay sensitivity or increased error rate. Some instruments have substantial capability to resolve detection of signal that is detected in multiple channels (e.g., through the use of optical filtering and/or software manipulation of collected signal), so selection of various combinations of dyes is related to the instrument to be used to detect the multiplexed reaction.

The fluorescence output of a given dye from a fluorescence plate reader scan is proportional to its concentration as follows:
Fluorescence=α·[dye]+b (1)
Where α is a constant that varies with the excitation and emission wavelengths and the gain settings of the plate reader and b is background. If multiple dyes are present, then each dye contributes to the total fluorescence as
Fluorescence=α·[dyeₐ]+β·[dye_{b}]+γ·[dye]+ . . . n·[dyeₙ]+background (2)
Or
Fluorescence−background=α·[dyeₐ]+β·[dye_{b}]+γ·[dye_{c}]+ . . . n·[dyeₙ] (3)

When multiple scans are made, the fluorescence from each scan can be written as such:
Fluorescence₁−background₁=α₁·[dyeₐ]+β₁·[dye_{b}]+γ₁·[dye_{c}]+ . . . n₁·[dyeₙ]
Fluorescence₂−background₂=α₂·[dyeₐ]+β₂·[dye_{b}]+γ₂·[dye_{c}]+ . . . n₂·[dyeₙ]
Fluorescence₃−background₃=α₃·[dyeₐ]+β₃·[dye_{b}]+γ₃·[dye_{c}]+ . . . n₃·[dyen]
Fluorescenceₙ−backgroundₙ=αₙ·[dyeₐ]+βₙ·[dye_{b}]+γₙ·[dye_{c}]+ . . . nₙ·[dyeₙ]
where the numerical subscripts represent the fluorescence readings, dye coefficients, and background component for each scan. This series of linear equations can be written in matrix form as

$$( \begin{matrix}{F}_{1} - {b}_{1} \\ {F}_{2} - {b}_{2} \\ {F}_{3} - {b}_{3} \\ ⋮ \\ {F}_{n} - {b}_{n}\end{matrix} ) = ( \begin{matrix}{α}_{1} & {β}_{1} & {γ}_{1} & {n}_{1} \\ {α}_{2} & {β}_{2} & {γ}_{2} & {n}_{2} \\ {α}_{3} & {β}_{3} & {γ}_{3} & {n}_{3} \\ ⋮ & ⋮ & ⋮ & ⋮ \\ {α}_{n} & {β}_{n} & {γ}_{n} & {n}_{n}\end{matrix} ) ( \begin{matrix}{dye}_{A} \\ {dye}_{B} \\ {dye}_{C} \\ ⋮ \\ {dye}_{N}\end{matrix} ) \qquad ( 3 )$$
Or
   F=Ad (4)
where the elements of the linear matrix F are the background subtracted fluorescence readings, A is the two-dimensional coefficient matrix, and d is the linear matrix whose elements are the amounts of each free dye released from the INVADER assay. The elements of the F and A matrices can be determined providing that there is some sort of calibration using pure dyes and blanks for each different scan. Therefore, the solution for d can be found by left multiplying both sides of equation (4) by the inverse of A.

Such a matrix was derived for the 4-plex dye set as follows. Dye-T10 oligonucleotides, i.e. oligos comprising 10 dT residues with a 5′ terminal dye, were used to determine emission characteristics of “free dye”. Different ratios of these dT10 oligos were combined with FRET probes comprising the corresponding dye and an appropriate quencher to mimic signal generation from the INVADER assay over time. Working stocks of 500 nM were made of each dT10 and each FRET probe, respectively. Total sample volumes were 15 μl, and each sample was overlaid with 15 μl mineral oil. Ratios tested were 0% dT10/100% FRET probe; 25% dT10/75% FRET probe; 50% dT10/50% FRET probe; 75% dT10/25% FRET probe; and 100% dT10/0% FRET probe. The dyes tested were fluorescein (FAM), Cal-Gold and Cal-Orange (Biosearch Technologies, Inc., Novato, Calif.), and REDMOND RED (Synthetic Genetics). Tubes were read in a Tecan Safire XFLUOR 4 at excitation and emission wavelengths appropriate for each dye. In each case, the fluorescence observed from each dye increased linearly with increasing proportions of dT10 oligo, and the signals were additive. The slopes from the linear regressions were entered into the coefficient matrix as follows.

| Scan | | | | |
|---|---|---|---|---|
| (Excitation/Emission) | FAM | GOLD | ORANGE | RED |
| 495 nm/520 nm | 9834 | 0 | 271 | 0 |
| (FAM) | | | | |
| 522 nm/543 nm | 478 | 9155 | 3658 | 0 |
| (GOLD) | | | | |
| 540 nm/561 nm | 0 | 1466 | 13796 | 0 |
| (ORANGE) | | | | |
| 575 nm/589 nm | 0 | 0 | 554 | 5444 |
| (RED) | | | | |

A corresponding matrix was generated by taking the inverse of each value to obtain A⁻¹, as described above and thus derive d, the percentage of free dye in each case.

INVADER assays were run as follows. Standard reactions were set up in a 15 pI final volume as described above with CLEAVASE VIII enzyme and 5 pM (final) synthetic target. Four different synthetic targets were used in the present example: wild-type and mutant for SNPs 1 and 2. The FRET probes used were as follows:

| | | | | Emission | |
|---|---|---|---|---|---|
| | | | | wavelength | |
| | Complete | X | | λ, max | |
| Arm sequence | sequence | (Quencher) | Y (Dye) | (nm) | Allele |
| 5′-cgcgccgaggn | 5′-Y-tct-X- | BHQ-1 | FAM | 520 | SNP 1 |
| | agccggttttccggctgagac | | | | mutant |
| | ctcggcgcg-hex | | | | |
| 5′-acggacgcggagn | 5′-Y-tct-X- | BHQ-1 | CAL | 543 | SNP 1 |
| | agccggttttc | | GOLD | | wild type |
| | cggctgagact | | | | |
| | ccgcgtccgt- | | | | |
| | hex | | | | |
| 5′-aggccacggacgn | 5′-Y-tct-X- | BHQ-1 | CAL | 561 | SNP 2 |
| | agccggttttc | | ORANGE | | wild type |
| | cggctgagac | | | | |
| | gtccgtggcct | | | | |
| | -hex | | | | |
| 5′- | 5′-Y-tct-X- | Z28 | REDMOND | 590 | SNP 2 |
| atgacgtggcagacn | agccggttttccggctgagagt | | RED | | mutant |
| | ctgccacgtcat-hex | | | | |

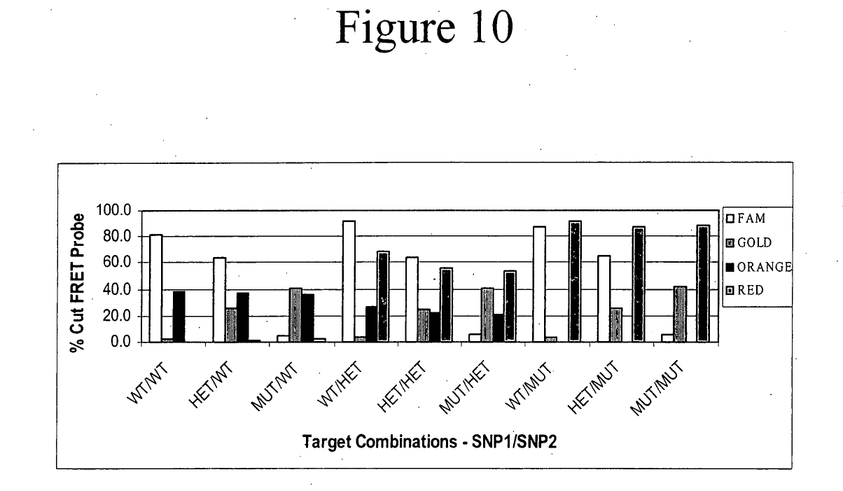

Assays were incubated at 63° C. and fluorescence read at the wavelengths indicated after 20 minutes. Results for these combined reactions are presented in FIG. 10, showing detection of the various combinations of target molecules.

### Example 10

### Microfluidic Card Pre-Loaded with Invader Assay Reagents for Target Detection

The following example described the use of a microfluidics card containing the INVADER assay reagents for interrogation of DNA samples. In this example, the target material has been prepared by prepared separately by PCR. The 3M microfluidic card has 8 loading ports, each of which is configured to supply liquid reagent to 48 individual reaction chambers upon centrifugation of the card. The reaction chambers contain pre-dispensed and dried INVADER assay reaction components for detection of one or more particular alleles (e.g. as shown in Example 11, below). These reagents are dissolved when they come in contact with the liquid reagents upon centrifugation of the card.

Multiplex PCR reaction mixtures were prepared using the following components (concentrations shown are at their final concentration in the PCR reaction): Genomic DNA at 2 ng/uL, multiplex PCR primer mix at 0.2 uM, PCR Buffer plus MgCl₂ at 1×, dNTPs at 0.2 mM, and native Taq polymerase at 0.2 U/rxn. The final reaction volume was 20 uL. These mixtures were heated for 2.5 min at 95 C, then were cycled 20 times through a 30 sec 95 C step, a 1.5 min 55 C step, and a 2.5 min 72 C step. Finally, the samples were incubated at 99 C for 10 min to destroy the polymerase activity.

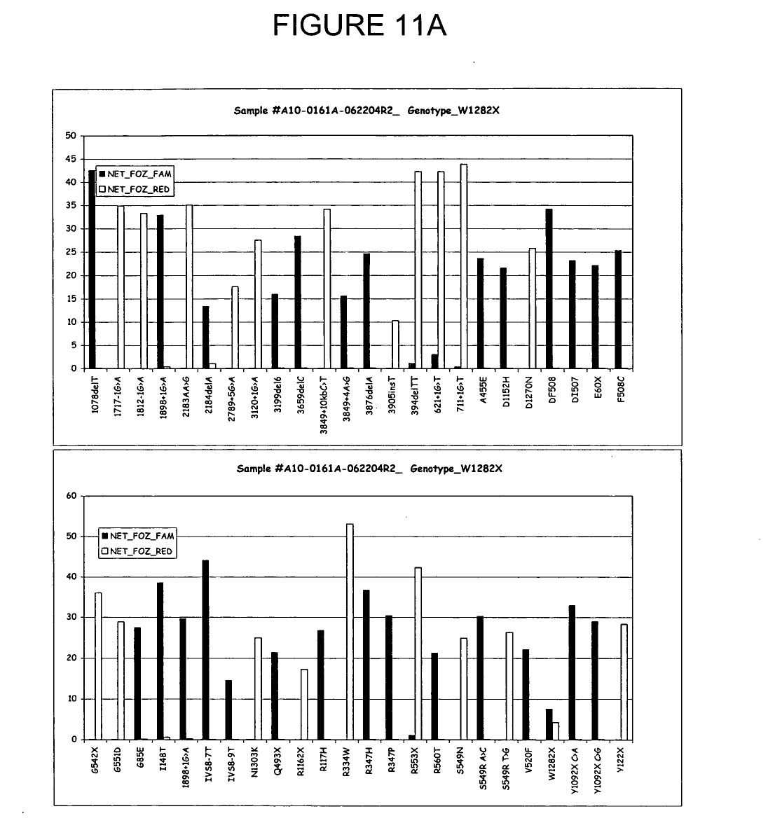

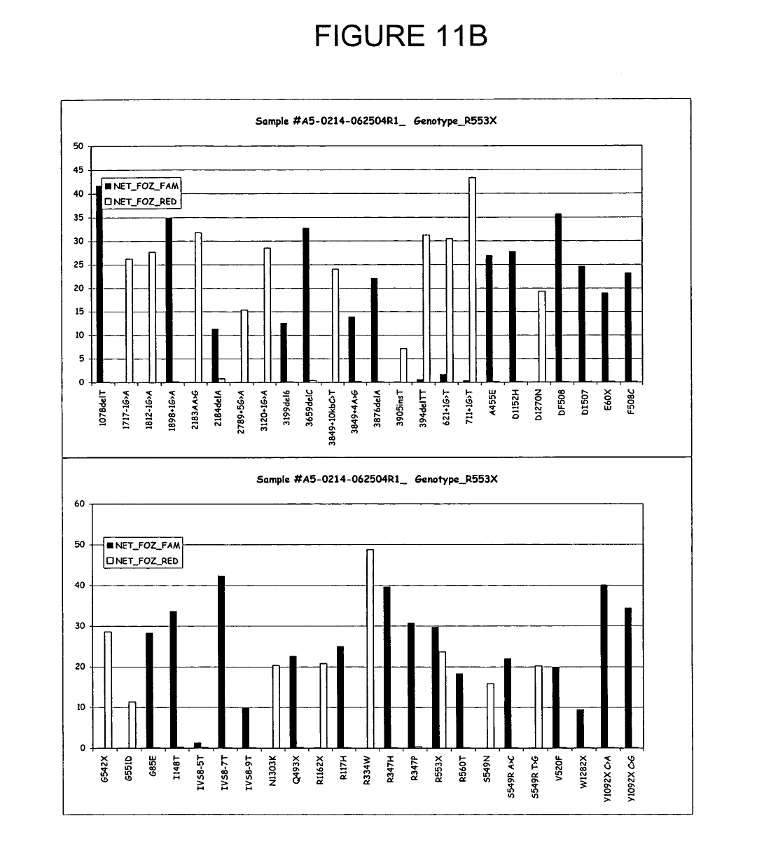

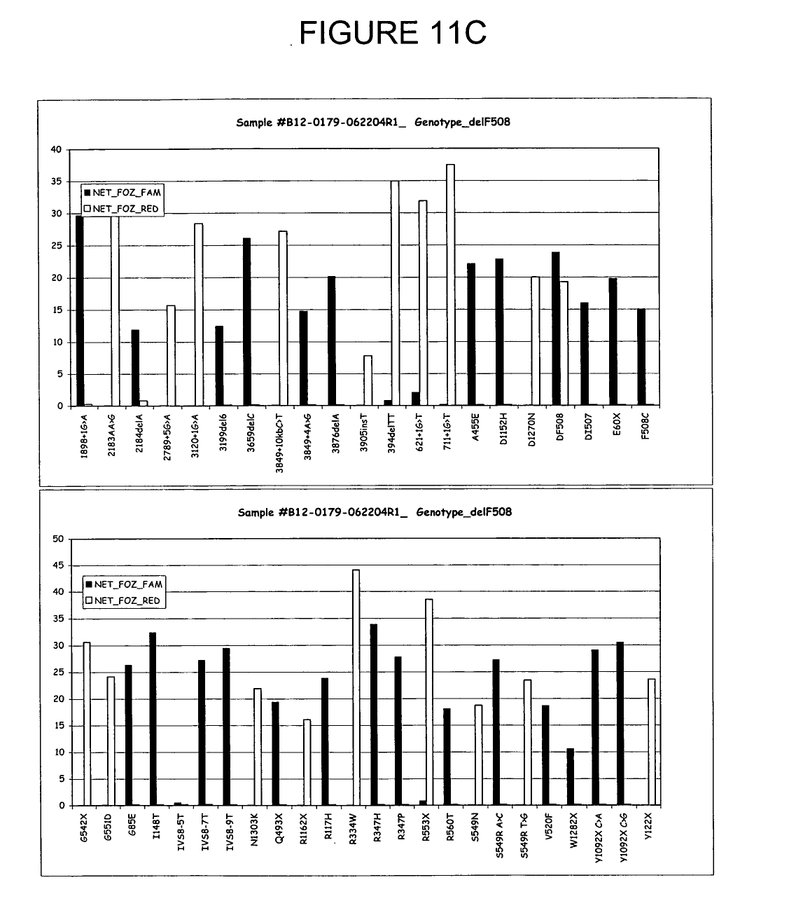

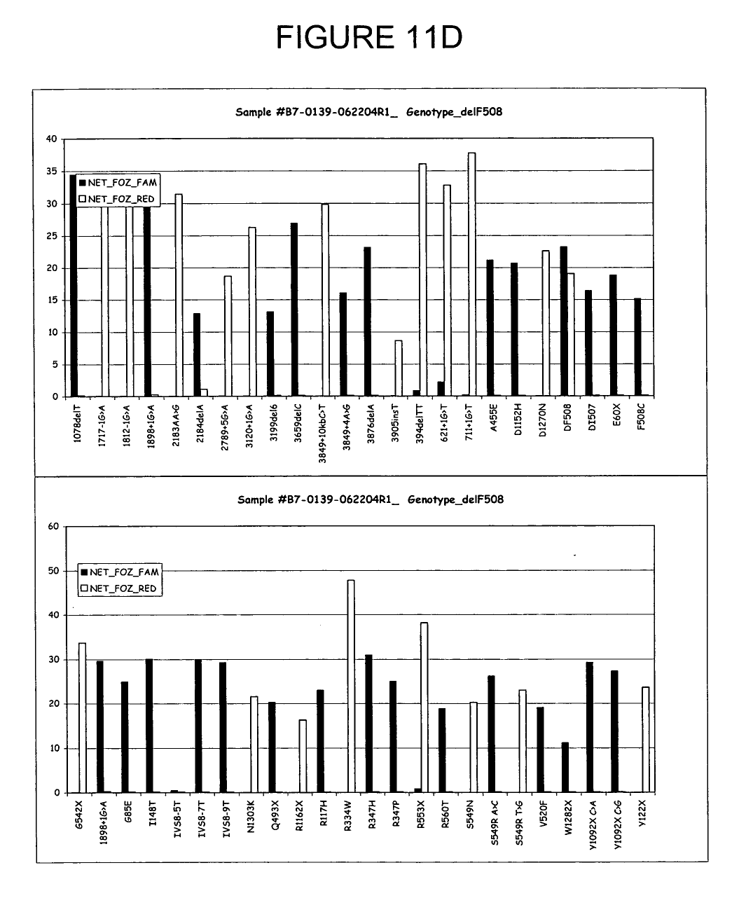

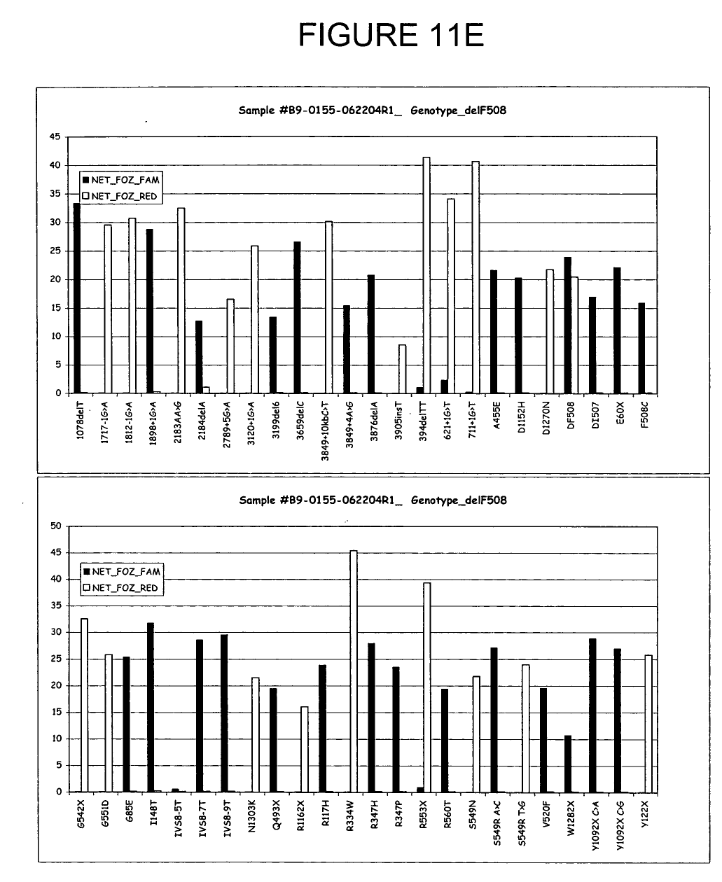

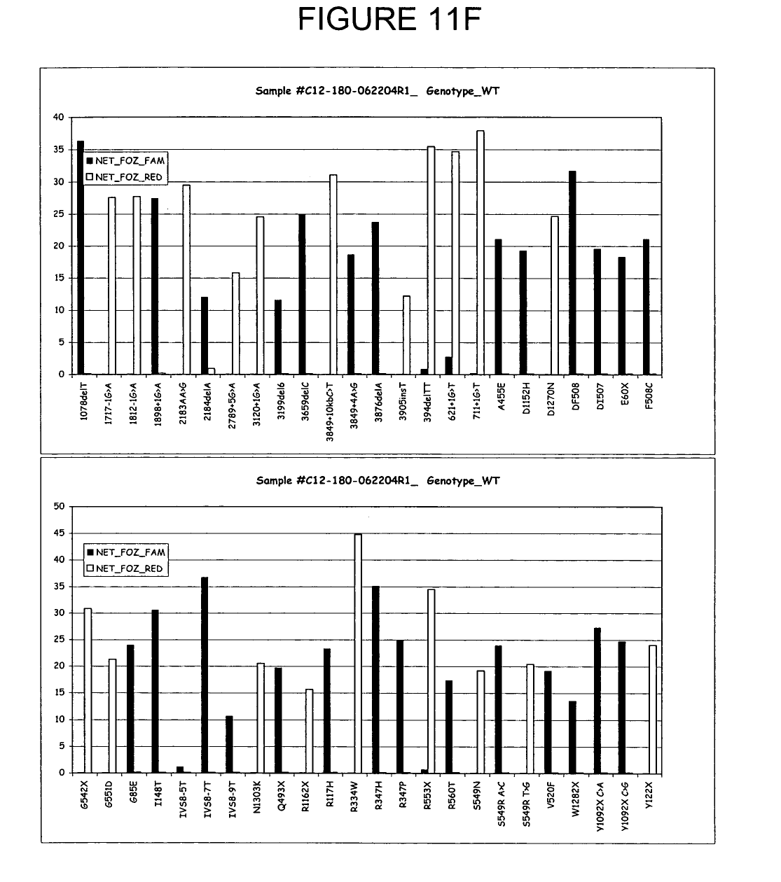

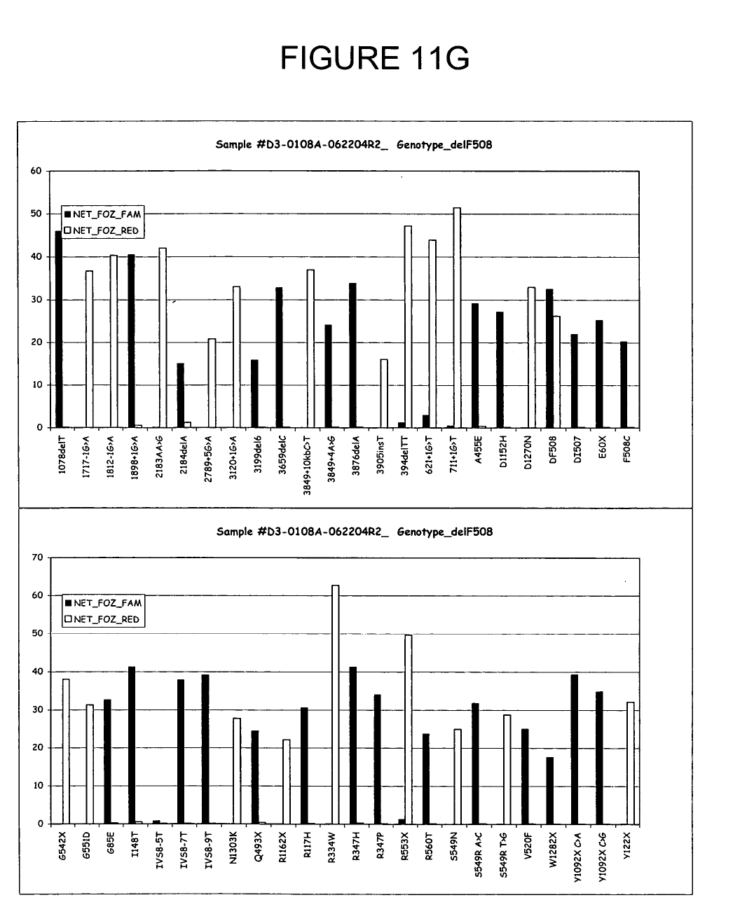

Following PCR, the amplicons were diluted 1:125 with dH₂O, and 50 uL of this sample was mixed with 50 μl of a solution containing 28 mM MgCl₂ and CLEAVASE X enzyme at 4 ng/μl. This mixture was then added to one of the 8 individual ports of the 3M CF microfluidics card described in the previous example. The INVADER assay was performed at 63 C for 20 min, and fluorescence from the assay was detected on a microplate fluorimeter. The results are shown in FIGS. 11A-11G. The genotype of the genomic sample DNA is indicated at the top of each panel, and each of the mutations tested is indicated along the X-axis.

### Example 11

### Invader Plus PCR on 3M CF Microfluidics Card

The following example described the use of a microfluidics card containing the INVADER assay reagents for interrogation of DNA samples. In this example, the target material is amplified and detected in a single reaction. The reactions were performed on a 3M microfluidic card, as described above.

The reaction chambers of the microfluidic card contain INVADER assay reaction components (i.e., the INVADER oligonucleotide, primary probe, and FRET cassettes) for running the 48 different INVADER assays dried down onto the card. To prepare such cards, 2 μl of 1× PPIFF-MOPS mix (0.25 μM each Primary Probe Oligonucleotide, 0.125 μM each FRET oligonucleotide, 0.025 μM INVADER oligonucleotide, in 10 mM MOPS buffer) is dispensed into the wells of the microfluidics card. The cards are then allowed to dry in an air box through which HEPA filtered air is forced. It generally not necessary to control temperature or relative humidity of the air. The volume of each reaction chamber in the assembled microfluidic card is about 1.7 uL, so the final concentrations of these components during the reaction are about 1.18 times those of the 1×PPIFF-MOPS mix).

The allelic variants detected by these INVADER assay oligonucleotide sets were as follows:

| | |
|---|---|
| 1 | E60X |
| 2 | G85E |
| 3 | 394delTT |
| 4 | R117H |
| 5 | Y122X |
| 6 | I148T |
| 7 | 621 + 1G > T |
| 8 | 711 + 1G > T |
| 9 | 1078delT |
| 10 | R334W |
| 11 | R347H |
| 12 | R347P |
| 13 | IVS8-5T |
| 14 | IVS8-7T |
| 15 | IVS8-9T |
| 16 | A455E |
| 17 | Q493X |
| 18 | DI507 |
| 19 | DF508 |
| 20 | F508C |
| 21 | V520F |
| 22 | 1717 − 1G > A |
| 23 | G542X |
| 24 | S549N |
| 25 | S549R A > C |
| 26 | S549R T > G |
| 27 | G551D |
| 28 | R553X |
| 29 | R560T |
| 30 | 1812 − 1G > A |
| 31 | 1898 + 1G > A |
| 32 | 2183AA > G |
| 33 | 2184delA |
| 34 | 2789 + 5G > A |
| 35 | 3120 + 1G > A |
| 36 | 3199del6 |
| 37 | Y1092X C > A |
| 38 | Y1092X C > G |
| 39 | D1152H |
| 40 | R1162X |
| 41 | 3659delC |
| 42 | 3876delA |
| 43 | D1270N |
| 44 | 3905insT |
| 45 | W1282X |
| 46 | N1303K |
| 47 | 3849 + 4A > G |
| 48 | 3849 + 10kbC > T |

A master mix containing all the materials necessary for a multiplex PCR amplifying the targets of the INVADER assay, along with the CLEAVASE VIII enzyme required for the INVADER assay, was prepared and split into 8 pools. To 7 of these 8 pools a unique sample of genomic DNA was added, and the remaining sample was used as a control that contained no template. 100 uL of each of these 8 mixtures was added to each loading port on the card, and the wells in the card were loaded by centrifugation. The final concentration of components in these mixtures was as follows: 7.5 mM MgCl₂, 6.67 ng/uL Cleavase VIII, 0.033 U/uL Native Taq-pol, 25 uM dNTP mix, 0.2 uM multiplex PCR primers.

The combined PCR and INVADER assay reactions were incubated as follows: 95 C for 15 sec and 72 C for 2 min 15 sec, for 15 cycles, followed by a single 99 C step for 10 min to destroy native Taq-pol activity, followed by 60 C for 1 hour for the INVADER assay reaction.

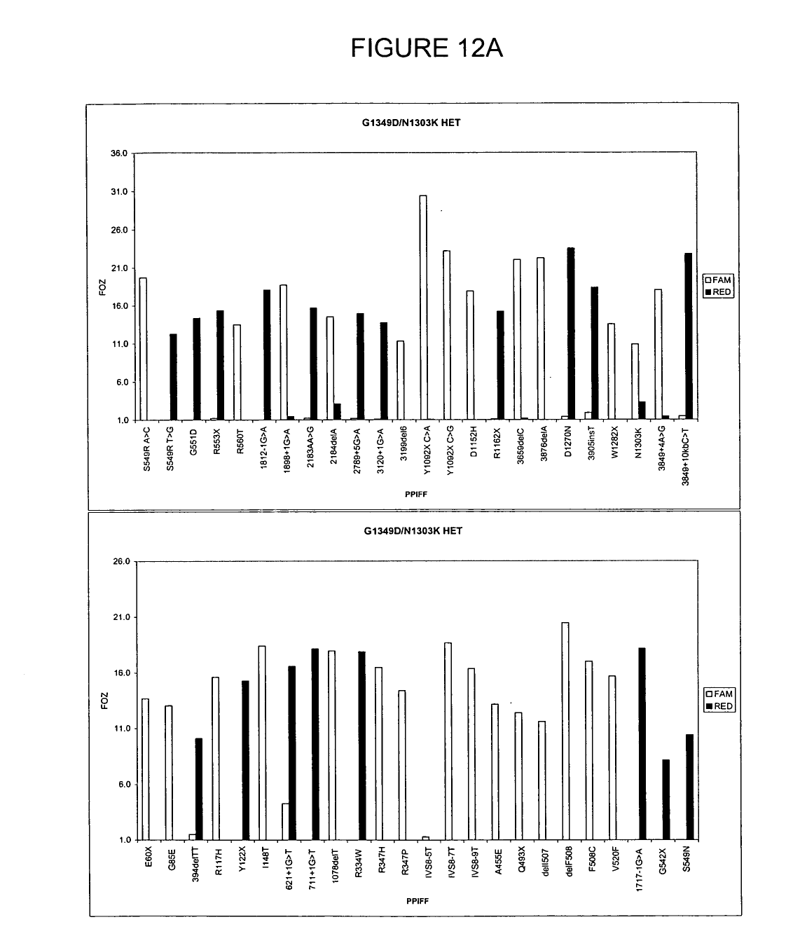

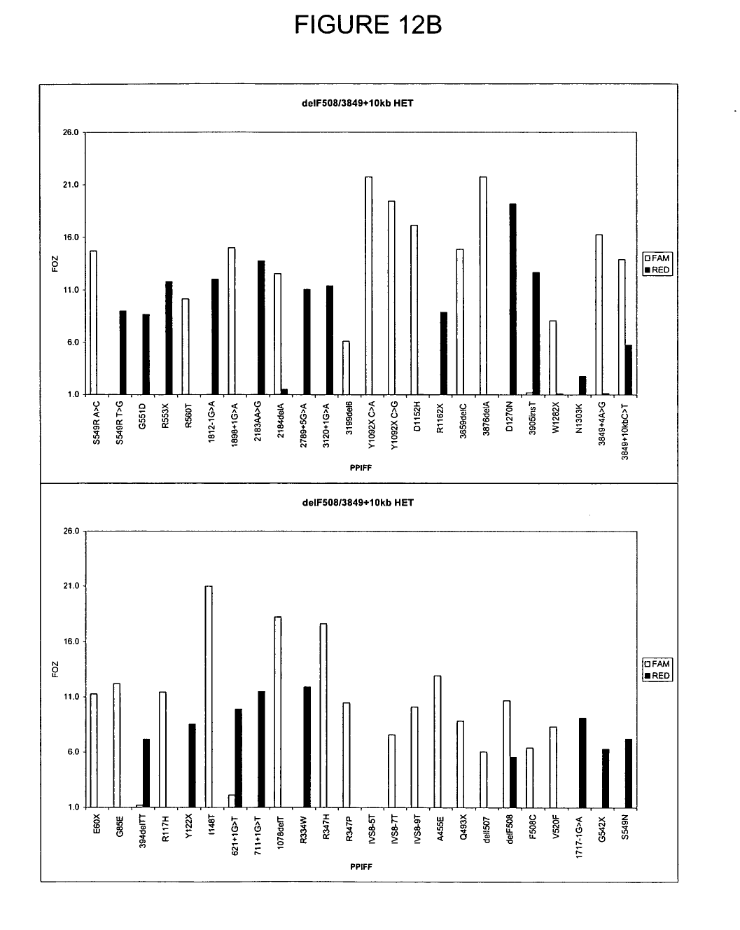

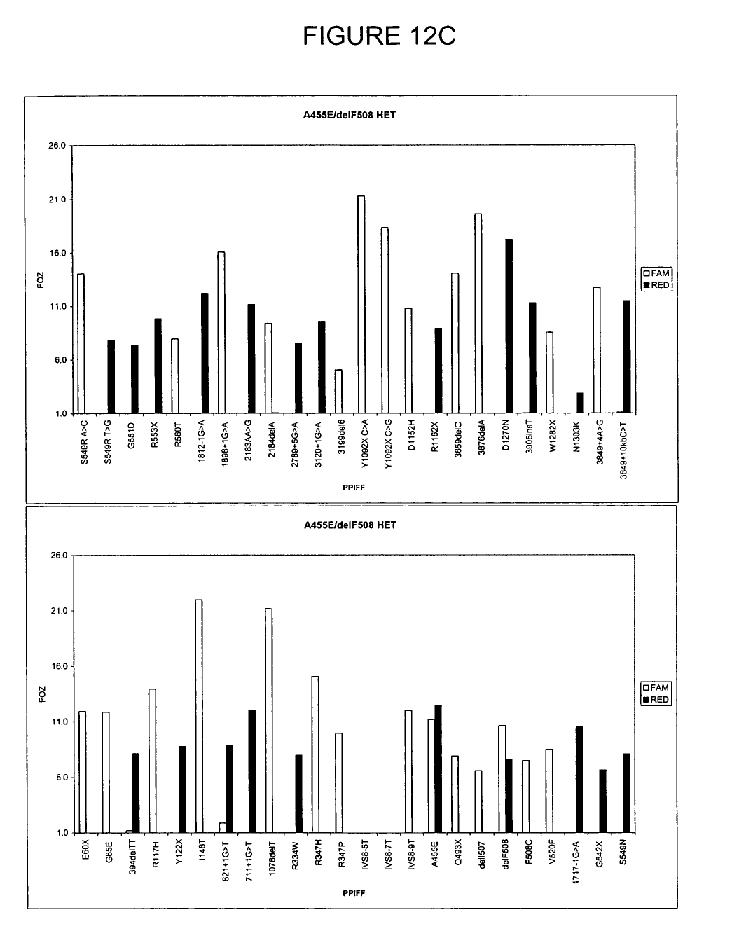

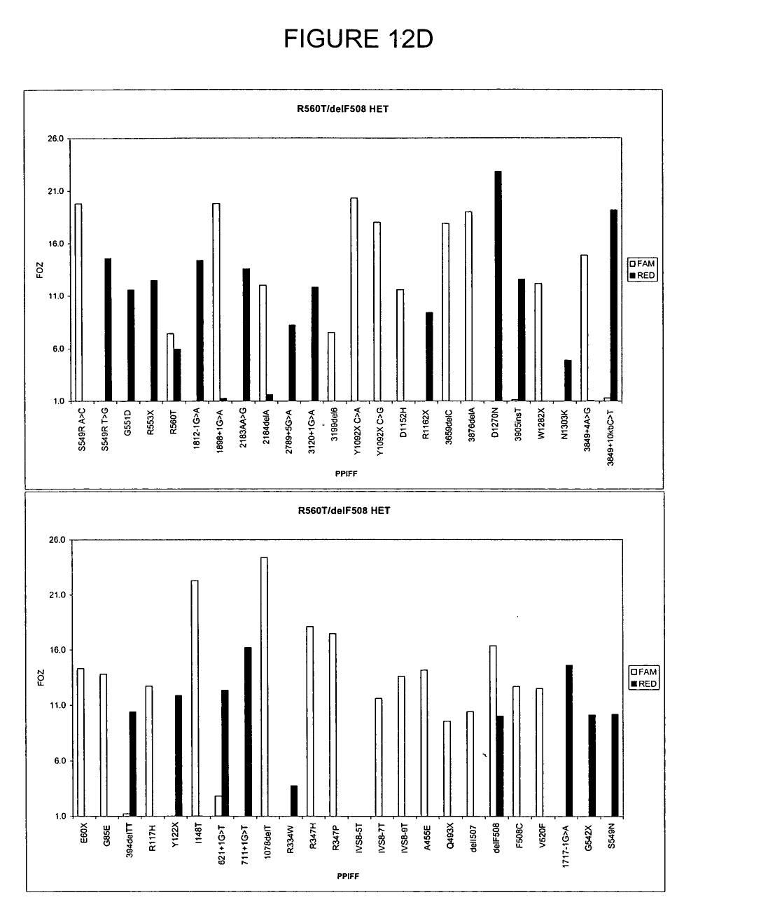

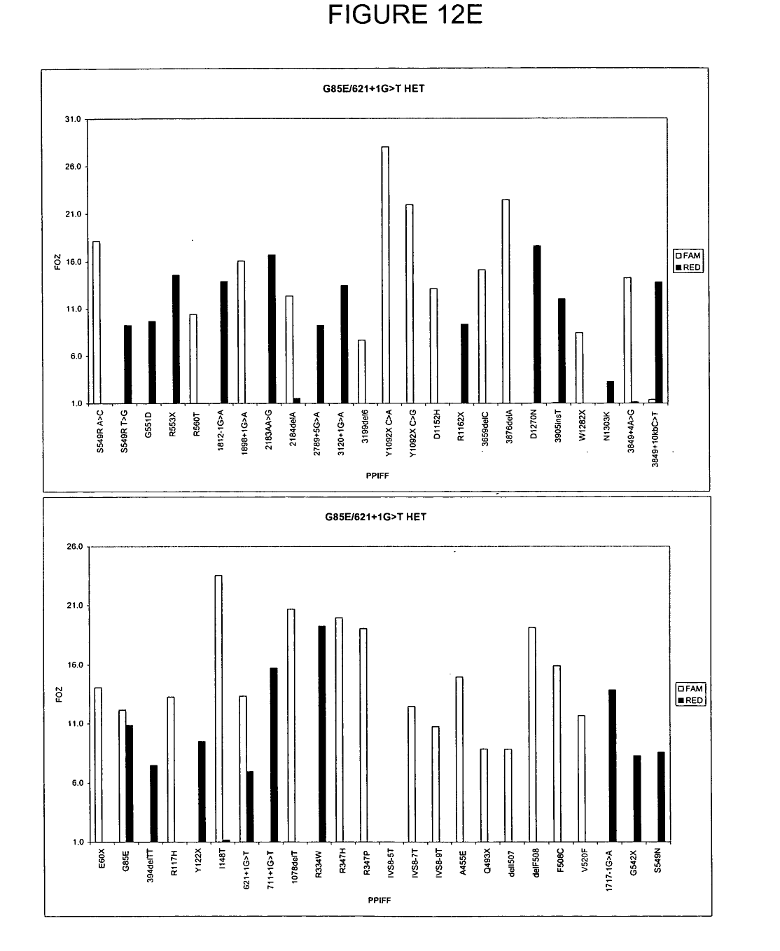

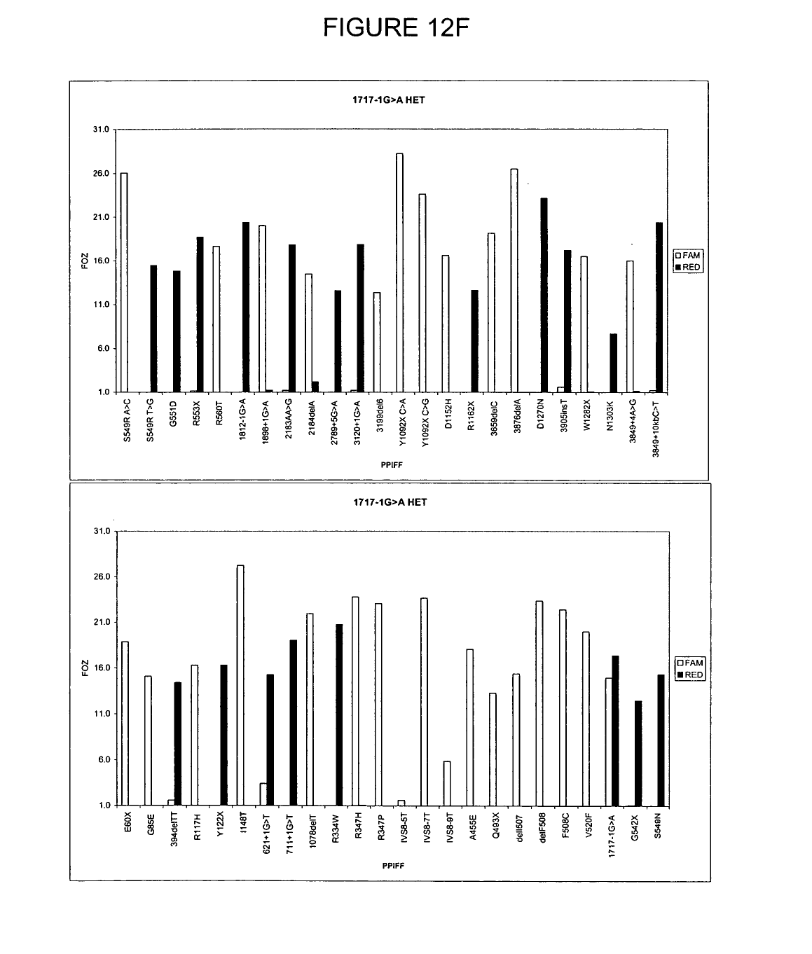

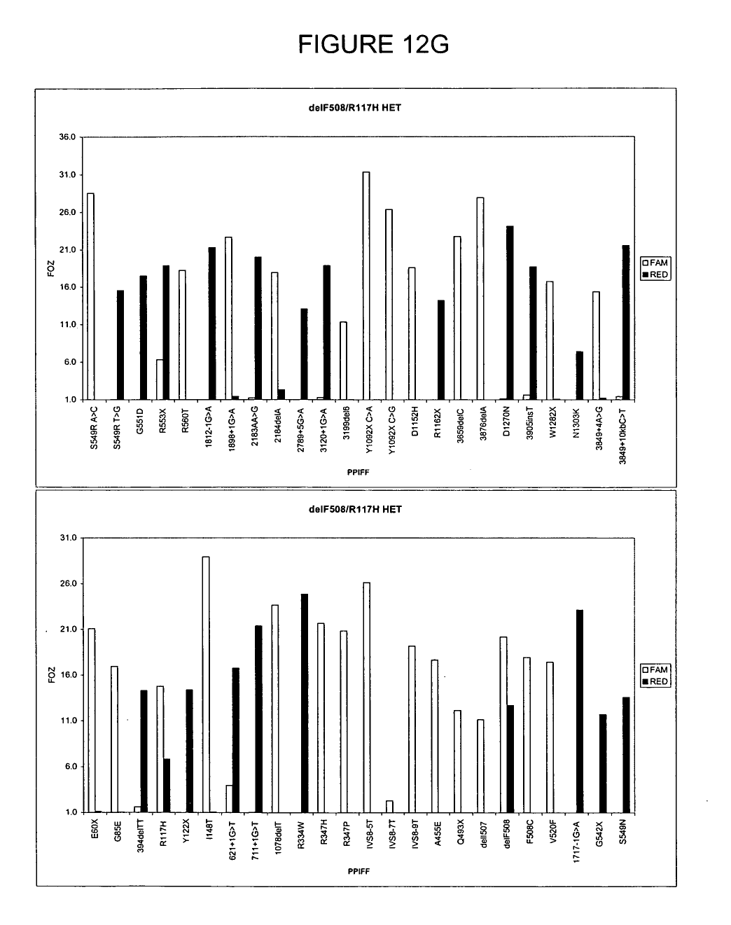

Fluorescent signal from the INVADER assay was detected on a fluorescent microplate reader, and the results are presented in FIGS. 12A-12G (the no-target control samples are omitted). The genotype of each genomic sample DNA is indicated at the top of each panel, and each of the mutations tested is indicated along the X-axis.

### Example 12

### Direct Detection in Un-Purified Whole Blood Samples

This Example describes the achievement of direct detection of a target sequence in an un-purified whole blood sample. In particular, this example, similar to the Examples above, describes the combination of PCR amplification and INVADER assay detection in a single reaction vessel to detect a genomic DNA. This Example, however, further extends the above Examples by applying the method of single reaction vessel, combined PCR-INVADER analysis, to an un-purified whole blood sample.

The PCR/INVADER assay reaction mixture, in a total volume of 20 ul, is prepared as follows. For the buffer, about 4 ul of either 0.5×AMPDIRECT-A from Shimadzu (without 5×Amp Addition-1) or 10 mM TAPS biological buffer (3-[[tris(Hydroxymethyl)methyl]amino]propanesulfonic acid) approximately pH 9 are employed.

It is noted that it was unexpected to find that TAPS pH 9, rather than just AMPDIRECT-A, will serve as the buffer for direct PCR and INVADER detection in whole blood. Also, additional details on the AMPDIRECT-A buffer and PCR in whole blood may be found, for example, in U.S. Patent Pubs 20020102660 and 20020142402, as well as Nishimura et al., Clin. Lab., 2002, 48:377-84, and Nishimura et al., Ann. Clin Biochem, 2000, 37:674-80, all of which are herein incorporated by reference for all purposes). The following additional reagents are used: 6.25 uM dNTPs each dNTP, 0.2 uM each PCR primer, 0.3 units of Taq polymerase (native), 40 ng of CLEAVASE VIII, 3 mM MgCl₂ (in addition to any MgCl₂ in the AMPDIRECT buffer, if this buffer is used), 0.5 uM Primary Probe for each target to be detected (e.g., for targeted genomic DNA and for internal control), 0.05 uM INVADER oligonucleotide for each allele to be detected (for use with multiple Primary Probes, if a SNP is to be detected) or 0.05 uM INVADER oligonucleotide for each target to be detected (for use, e.g., when quantitating a variable target against an internal control target) 0.25 uM each FRET probe (for target and control reactions), and distilled water for a total reaction volume of 20 uL.

The liquid whole human blood sample to be tested is first treated with an anticoagulant, such as sodium citrate, dipotassium EDTA, or sodium heparinate. About 0.4 ul (or less) of this treated whole human blood is added to the PCR/INVADER reaction mixture by loading it to the bottom of the reaction tube without mixing. Mineral oil can be overlayed if needed. Next, PCR is carried out on the sample for a total of 28 cycles. PCR can be carried out, for example, using the following temperature profile, which is suitable for whole human blood: preheating at 80 C for 15 min, then 94 C for 4.5 min, followed by 28 cycles of 94 C for 30 seconds, annealing temperature for 1 minute, 72 C for 1 minute, and 72 C for 7 minutes.

Following these cycling reactions, the mixture is heated to 99° C. for 10 minutes to inactivate the Taq DNA polymerase. The reaction mixture is then incubated at 63° C. for about 30 minutes to about 3 hours to allow the INVADER assay reactions to proceed. Results from the INVADER assay are collected (see, e.g., the Examples described above). The results of this example show successful PCR amplification of a target sequence in genomic DNA within the whole blood, as wells as successful INVADER assay detection of the target sequence of interest. Success in detecting the target nucleic acids of interest from this whole blood is possible whether AMPDIRECT or TAPS Ph 9 is used as the buffer.

Direct DNA detection with combined PCR and INVDADER assays may also be performed using blood-spot cards, such as those from WHATMAN. The PCR-INVADER reaction buffer, similar to the above, can be prepared as follows: 10 mM TAPS pH 9 buffer, 3 mM MgCl₂, 0.2 uM of each PCR primer, 6.25 uM each dNTP, 0.5 uM Primary Probe for each target to be detected (e.g., for targeted genomic DNA and for internal control), 0.05 uM INVADER oligonucleotide for each allele to be detected (for use with multiple Primary Probes, if a SNP is to be detected) or 0.05 uM INVADER oligonucleotide for each target to be detected (for use, e.g., when quantitating a variable target against an internal control target) 0.25 uM each FRET probe (for target and control reactions), 0.06 ul of TaqPol (native, 5 u/ul), 0.2 ul of CLEAVASE VIII 200 ng/ul, and distilled water for final volume of 20 ul.

From a WHATMAN FTA Gene card spotted with blood, one 1 millimeter punch is taken that contains the blood, and one control punch of the same diameter is taken from a location on the card without any blood. The paper punches are then washed in 1 ml of water for about 10 minutes, with occasional rocking to stir.

PCR and INVADER assays are performed as described above. The results of this procedure show successful PCR amplification of the target sequence from genomic within the whole blood, as well as successful INVADER assay detection of the target sequence of interest.

All publications and patents mentioned in the above specification are herein incorporated by reference as if expressly set forth herein. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in relevant fields are intended to be within the scope of the following claims.

1. A method for detecting a target nucleic acid in unpurified bodily fluids comprising: exposing an unpurified bodily fluid to detection assay reagents under conditions such that said target nucleic acid is detected, if present, in a single step reaction.

2. The method of claim 1, wherein said single step reaction comprises a polymerase chain reaction.

3. The method of claim 1, wherein said single step reaction comprises a polymerase chain reaction and an invasive cleavage assay reaction.

4. The method of claim 1, wherein said single step reaction comprises an invasive cleavage assay reaction.

5. The method of claim 3, wherein said polymerase chain reaction has less than 20 amplification cycles.

6. The method of claim 3, wherein said polymerase chain reaction has less than 15 amplification cycles.

7. The method of claim 3, wherein said polymerase chain reaction has less than 12 amplification cycles.

8. The method of claim 1, wherein said target nucleic acid is mammalian genomic DNA.

9. The method of claim 1, wherein said target nucleic acid is from a pathogen.

10. The method of claim 1, wherein said target nucleic acid is from a plant.

11. The method of claim 1, wherein said fluid comprises blood.

12. The method of claim 1, wherein said target nucleic acid is detected by fluorescence.

13. The method of claim 1, wherein said reagents comprise a polymerase, a 5′ nuclease, and a buffer.

14. The method of claim 13, wherein said buffer comprises pH 9 TAPS.

15. A kit for detecting a target nucleic acid in unpurified bodily fluids comprising: a polymerase, a 5′ nuclease, and a buffer that permits detectable amplification of said target nucleic acid in an unpurified bodily fluid.

16. The kit of claim 15, wherein said 5′ nuclease comprises a FEN-1 endonuclease.

17. The kit of claim 15, wherein said buffer comprises pH 9 TAPS.

18. The kit of claim 15, further comprising amplification primers.

19. The kit of claim 15, further comprising oligonucleotides configured to create an invasive cleavage structure in the presence of said target nucleic acid.

20. A method for multiplex detection of target nucleic acids, comprising: a) providing polymerase chain reaction and invasive cleavage assay reagents in a microfluidics card, wherein said reagents are configured to amplify and detect said target nucleic acids; b) exposing a sample suspected of containing said target nucleic acids to said reagents using centrifugal force; and c) detecting the presence or absence of said target nucleic acids.

21. The method of claim 20, wherein said exposing comprising conducting 20 or less cycles of polymerase chain reaction.

22. The method of claim 20, wherein said reagents comprise a polymerase and a 5′ nuclease.

23. The method of claim 22, wherein said 5′ nuclease comprises a FEN-1 endonuclease.


SEQUENCE LISTING (72 sequences)

SEQ ID NO: 1 - DNA, 33 bp, Artificial Sequence
  Synthetic
cccgacatttttacgcatgcgcaaactccaacc

SEQ ID NO: 2 - DNA, 29 bp, Artificial Sequence
  Synthetic
tacacgcacgcgcaagaagcaagaggact

SEQ ID NO: 3 - DNA, 28 bp, Artificial Sequence
  Synthetic
ctgggtttccaacaggcgaaaaggccct

SEQ ID NO: 4 - DNA, 28 bp, Artificial Sequence
  Synthetic
gcgtgagggtggaaggagatgcccatgg

SEQ ID NO: 5 - DNA, 28 bp, Artificial Sequence
  Synthetic
acggacgcggagaggaaccctgtgacat

SEQ ID NO: 6 - DNA, 25 bp, Artificial Sequence
  Synthetic
ccatccagggaagagtggcctgttt

SEQ ID NO: 7 - DNA, 24 bp, Artificial Sequence
  Synthetic
cgcgccgaggttctgcgcatgtgc

SEQ ID NO: 8 - DNA, 25 bp, Artificial Sequence
  Synthetic
agggagagccgtgcaccacgatgac

SEQ ID NO: 9 - DNA, 33 bp, Artificial Sequence
  Synthetic
tctagccggttttccggctgagacctcggcgcg

SEQ ID NO: 10 - DNA, 35 bp, Artificial Sequence
  Synthetic
tcttcggccttttggccgagagactccgcgtccgt

SEQ ID NO: 11 - DNA, 10 bp, Artificial Sequence
  Synthetic
cgcgccgagg

SEQ ID NO: 12 - DNA, 14 bp, Artificial Sequence
  Synthetic
atgacgtggcagac

SEQ ID NO: 13 - DNA, 12 bp, Artificial Sequence
  Synthetic
acggacgcggag

SEQ ID NO: 14 - DNA, 11 bp, Artificial Sequence
  Synthetic
tccgcgcgtcc

SEQ ID NO: 15 - DNA, 22 bp, Artificial Sequence
  Synthetic
gaagcggcgccggttaccacca

SEQ ID NO: 16 - DNA, 27 bp, Artificial Sequence
  Synthetic
cgcgccgaggtggttgagcaattccaa

SEQ ID NO: 17 - DNA, 30 bp, Artificial Sequence
  Synthetic
atgacgtggcagaccggttgagcaattcca

SEQ ID NO: 18 - DNA, 27 bp, Artificial Sequence
  Synthetic
tggtcccactttttattcttttgcaga

SEQ ID NO: 19 - DNA, 22 bp, Artificial Sequence
  Synthetic
aagtcaccaaagcagtacagcc

SEQ ID NO: 20 - DNA, 25 bp, Artificial Sequence
  Synthetic
gctgtcaagccgtgttctagataaa

SEQ ID NO: 21 - DNA, 21 bp, Artificial Sequence
  Synthetic
cggaaggcagcctatgtgaga

SEQ ID NO: 22 - DNA, 23 bp, Artificial Sequence
  Synthetic
catgggccatgtgcttttcaaac

SEQ ID NO: 23 - DNA, 23 bp, Artificial Sequence
  Synthetic
catgggccatgtgcttttcaaac

SEQ ID NO: 24 - DNA, 26 bp, Artificial Sequence
  Synthetic
cttcttggtactcctgtcctgaaaga

SEQ ID NO: 25 - DNA, 24 bp, Artificial Sequence
  Synthetic
attatgggagaactggagccttca

SEQ ID NO: 26 - DNA, 31 bp, Artificial Sequence
  Synthetic
gattacattagaaggaagatgtgcctttcaa

SEQ ID NO: 27 - DNA, 29 bp, Artificial Sequence
  Synthetic
taaggcaaatcatctacactagatgacca

SEQ ID NO: 28 - DNA, 26 bp, Artificial Sequence
  Synthetic
taactgagaccttacaccgtttctca

SEQ ID NO: 29 - DNA, 28 bp, Artificial Sequence
  Synthetic
atgggaggaataggtgaagatgttagaa

SEQ ID NO: 30 - DNA, 24 bp, Artificial Sequence
  Synthetic
tctgaatgcgtctactgtgatcca

SEQ ID NO: 31 - DNA, 23 bp, Artificial Sequence
  Synthetic
cctgcacaatgtgcacatgtacc

SEQ ID NO: 32 - DNA, 21 bp, Artificial Sequence
  Synthetic
ggactatggacacttcgtgcc

SEQ ID NO: 33 - DNA, 29 bp, Artificial Sequence
  Synthetic
ggagaaggaagagttggtattatcctgac

SEQ ID NO: 34 - DNA, 29 bp, Artificial Sequence
  Synthetic
gcatcaaactaattgtgaaattgtctgcc

SEQ ID NO: 35 - DNA, 29 bp, Artificial Sequence
  Synthetic
gcatcaaactaattgtgaaattgtctgcc

SEQ ID NO: 36 - DNA, 28 bp, Artificial Sequence
  Synthetic
gaaggtggaaatgccatattagagaaca

SEQ ID NO: 37 - DNA, 28 bp, Artificial Sequence
  Synthetic
gtacctatatgtcacagaagtgatccca

SEQ ID NO: 38 - DNA, 29 bp, Artificial Sequence
  Synthetic
gattagaaaaatgttcacaagggactcca

SEQ ID NO: 39 - DNA, 28 bp, Artificial Sequence
  Synthetic
cagttgacttgtcatcttgatttctgga

SEQ ID NO: 40 - DNA, 23 bp, Artificial Sequence
  Synthetic
cctcgacaatgtgcacatgtacc

SEQ ID NO: 41 - DNA, 30 bp, Artificial Sequence
  Synthetic
acctattcaccagatttcgtagtcttttca

SEQ ID NO: 42 - DNA, 29 bp, Artificial Sequence
  Synthetic
tgtaccagctcactacctaatttatgaca

SEQ ID NO: 43 - DNA, 29 bp, Artificial Sequence
  Synthetic
gagctgagcaagacttaaccactaattac

SEQ ID NO: 44 - DNA, 28 bp, Artificial Sequence
  Synthetic
gtgaacattcctagtattagctggcaac

SEQ ID NO: 45 - DNA, 28 bp, Artificial Sequence
  Synthetic
ctccaaaaataccttccagcactacaaa

SEQ ID NO: 46 - DNA, 29 bp, Artificial Sequence
  Synthetic
gaaattactgaagaagaggctgtcatcac

SEQ ID NO: 47 - DNA, 28 bp, Artificial Sequence
  Synthetic
ctccaaaaataccttccagcactacaaa

SEQ ID NO: 48 - DNA, 27 bp, Artificial Sequence
  Synthetic
gactaaccgattgaatatggagccaaa

SEQ ID NO: 49 - DNA, 29 bp, Artificial Sequence
  Synthetic
cttaaatgtgattcttaacccactagcca

SEQ ID NO: 50 - DNA, 28 bp, Artificial Sequence
  Synthetic
gaggtaaaatgcaatctatgatgggaca

SEQ ID NO: 51 - DNA, 27 bp, Artificial Sequence
  Synthetic
taagggagtcttttgcacaatggaaaa

SEQ ID NO: 52 - DNA, 24 bp, Artificial Sequence
  Synthetic
acctcacccaactaatggtcatca

SEQ ID NO: 53 - DNA, 25 bp, Artificial Sequence
  Synthetic
tagacaggacttcaaccctcaatca

SEQ ID NO: 54 - DNA, 26 bp, Artificial Sequence
  Synthetic
gagtatcgcacattcactgtcatacc

SEQ ID NO: 55 - DNA, 29 bp, Artificial Sequence
  Synthetic
aaggtaacagcaatgaagaagatgacaaa

SEQ ID NO: 56 - DNA, 27 bp, Artificial Sequence
  Synthetic
taatgacagatacacagtgaccctcaa

SEQ ID NO: 57 - DNA, 22 bp, Artificial Sequence
  Synthetic
gcttcaggctactgggattcac

SEQ ID NO: 58 - DNA, 29 bp, Artificial Sequence
  Synthetic
gtcatctttcttcacgtgtgaattctcaa

SEQ ID NO: 59 - DNA, 22 bp, Artificial Sequence
  Synthetic
gcttcaggctactgggattcac

SEQ ID NO: 60 - DNA, 24 bp, Artificial Sequence
  Synthetic
ttctggctaagtccttttgctcac

SEQ ID NO: 61 - DNA, 25 bp, Artificial Sequence
  Synthetic
catttcagttagcagccttacctca

SEQ ID NO: 62 - DNA, 24 bp, Artificial Sequence
  Synthetic
tcctccctgagaatgttggatcaa

SEQ ID NO: 63 - DNA, 26 bp, Artificial Sequence
  Synthetic
tgatggtggtatgttttcaggctaga

SEQ ID NO: 64 - DNA, 24 bp, Artificial Sequence
  Synthetic
gttctcccctgtcccagttttaac

SEQ ID NO: 65 - DNA, 11 bp, Artificial Sequence
  Synthetic
cgcgccgaggn

SEQ ID NO: 66 - DNA, 33 bp, Artificial Sequence; identical to SEQ ID NO: 9
  Synthetic

SEQ ID NO: 67 - DNA, 13 bp, Artificial Sequence
  Synthetic
acggacgcggagn

SEQ ID NO: 68 - DNA, 35 bp, Artificial Sequence
  Synthetic
tctagccggttttccggctgagactccgcgtccgt

SEQ ID NO: 69 - DNA, 13 bp, Artificial Sequence
  Synthetic
aggccacggacgn

SEQ ID NO: 70 - DNA, 35 bp, Artificial Sequence
  Synthetic
tctagccggttttccggctgagacgtccgtggcct

SEQ ID NO: 71 - DNA, 15 bp, Artificial Sequence
  Synthetic
atgacgtggcagacn

SEQ ID NO: 72 - DNA, 37 bp, Artificial Sequence
  Synthetic
tctagccggttttccggctgagagtctgccacgtcat